(19) United States
(12) Patent Application Publication
(10) Pub. No.: US 2023/0399393 A1
(43) Pub. Date: Dec. 14, 2023

(54) METHODS TO EXTEND HEALTH-SPAN AND TREAT AGE-RELATED DISEASES

(71) Applicants: National University of Singapore, Singapore (SG); Singapore Health Services PTE LTD., Singapore (SG)

(72) Inventors: Benjamin Corden, Singapore (SG); Anissa Widjaja, Singapore (SG); Sebastian Schaeffer, Singapore (SG); Stuart Cook, Singapore (SG); Brijesh Kumar Singh, Singapore (SG)

(73) Assignees: National University of Singapore, Singapore (SG); Singapore Health Services PTE LTD., Singapore (SG)

(21) Appl. No.: 18/034,672

(22) PCT Filed: Oct. 29, 2021
(86) PCT No.: PCT/EP2021/080202

§ 371 Date: Apr. 28, 2023

(30) Foreign Application Priority Data
Oct. 30, 2020 (GB) 2017244.1

(51) Int. Cl. C07K 16/24; A61P 43/00

(52) U.S. Cl. CPC C07K 16/244; A61P 43/00; C07K 2317/76; A61K 2039/505

(57) ABSTRACT

The present disclosure relates to the diagnosis, treatment and prophylaxis of age-related diseases, and increasing healthspan. Provided are methods of treating or preventing an age-related disease/condition, and in particular of treating or preventing frailty, comprising administering a therapeutically or prophylactically effective amount of an agent capable of inhibiting interleukin 11 (IL-11)-mediated signalling to a subject. Also provided are agents capable of inhibiting interleukin 11 (IL-11)-mediated signalling for use in such a method. Further provided is the use of agents capable of inhibiting interleukin 11 (IL-11)-mediated signalling in the manufacture of a medicament for use in such a method.

### TECHNICAL FIELD

The present disclosure relates to the diagnosis, treatment and prophylaxis of age-related diseases, and increasing healthspan.

### BACKGROUND

Age-associated decline in function is common across multiple organs and physiological systems and results in generally increased vulnerability to stressors, loss of resilience and frailty. The manifestation of frailty is a major factor in reducing healthspan (i.e. the period of an organisms' life during which they are in good health). A significant contributor to frailty is age-related loss of muscle mass with concomitant gain in fat mass. The accumulation of senescent cells increased reactive oxygen species and sterile inflammation are important pathologies in the aging process.

The key pathologies underlying ageing have been categorised as the “Hallmarks of Ageing”, of which there are nine (López-Otín et al. 2013)—specifically telomere attrition, genomic instability, mitochondrial dysfunction, cellular senescence, stem cell exhaustion, loss of proteostasis, deregulated nutrient sensing, epigenetic alterations, and altered intercellular communication. Of these, the Hallmarks with the most robust evidence they can be manipulated, genetically or therapeutically, to reduce ageing-related diseases and/or to extend lifespan include nutrient sensing (Liu and Sabatini 2020), loss of proteostasis (Kaushik and Cuervo 2015) and cellular senescence (Dolgin 2020). Altered intercellular communication, particularly increased chronic inflammation and IL6 upregulation, is also an important Hallmark that may be targeted to prevent, treat and/or reverse ageing processes (Furman et al. 2019; Ferrucci and Fabbri 2018; Ershler and Keller 2000).

The majority of data on the pathways and key genes that drive, one or more of, the Hallmarks of Ageing have been derived from genetic studies in worms, flies and mice. Data across species point to the critical role for the insulin/IGF-1 signaling (IIS), mammalian target of rapamycin 1 (mTORC1), AMP-activated kinase (AMPK) and MEK/ERK pathway (Slack et al. 2015; Liu and Sabatini 2020; Burkewitz, Zhang, and Mair 2014). While targeting senescence alone has been attempted, this does not address other Hallmarks and has no impact on the main ageing pathways (IIS, ERK, mTORC1, AMPK).

Therapeutic targeting of IIS at the level of insulin, its receptor or IRS1 is associated with major toxicities. Inhibition of mTORC downstream of IIS, nutrient sensing and other ageing-related processes is possible using the immunosuppressant rapamycin that has a lesser, but still notable, toxicity profile. Across species, rapamycin and rapamycin-like drugs (rapalogs) improve healthspan and can extend lifespan (Zhang, Zhang, and Wang 2021; Fernandes and Demetriades 2021). Activation of AMPK using metformin is an alternative approach, which has successfully been used to improve healthspan and prolong lifespan (Burkewitz, Zhang, and Mair 2014). Of note, metformin depletes cellular energy stores and activates the nutrient sensing LKB1 kinase upstream of AMPK, rather than directly activating AMPK itself (Shaw et al. 2005). Experiments in flies have shown that therapeutic inhibition of MEK/ERK signaling with trimetinib can prolong lifespan (Slack et al. 2015). Overall, therapeutic interventions to date target individual pathways involved in ageing and are associated with major side effects. No drug inhibits all pathways.

There is an unmet clinical need for the development of therapies for the treatment and prevention of age-related diseases and conditions.

### SUMMARY

In a first aspect, the present disclosure provides a method of treating or preventing an age-related disease/condition, comprising administering a therapeutically or prophylactically effective amount of an agent capable of inhibiting interleukin 11 (IL-11)-mediated signalling to a subject, wherein the age-related disease/condition is selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

The present disclosure also provides an agent capable of inhibiting interleukin 11 (IL-11)-mediated signalling for use in a method of treating or preventing an age-related disease/condition is selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

The present disclosure also provides the use of an agent capable of inhibiting interleukin 11 (IL-11)-mediated signalling in the manufacture of a medicament for use in a method of treating or preventing an age-related disease/condition selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

The present disclosure also provides an agent capable of inhibiting interleukin 11 (IL-11)-mediated signalling for use in a method of treating or preventing frailty.

The present disclosure also provides the use of an agent capable of inhibiting interleukin 11 (IL-11)-mediated signalling in the manufacture of a medicament for use in a method of treating or preventing frailty.

The present disclosure also provides a method of treating or preventing frailty, comprising administering a therapeutically or prophylactically effective amount of an agent capable of inhibiting interleukin 11 (IL-11)-mediated signalling to a subject.

The present disclosure also provides an agent capable of inhibiting interleukin 11 (IL-11)-mediated signalling for use in a method of treating or preventing an age-related change in body composition.

The present disclosure also provides the use of an agent capable of inhibiting interleukin 11 (IL-11)-mediated signalling in the manufacture of a medicament for use in a method of treating or preventing an age-related change in body composition.

The present disclosure also provides a method of treating or preventing an age-related change in body composition, comprising administering a therapeutically or prophylactically effective amount of an agent capable of inhibiting interleukin 11 (IL-11)-mediated signalling to a subject.

The present disclosure also provides an agent capable of inhibiting interleukin 11 (IL-11)-mediated signalling for use in a method of increasing the healthspan of a subject.

The present disclosure also provides the use of an agent capable of inhibiting interleukin 11 (IL-11)-mediated signalling in the manufacture of a medicament for use in a method of increasing the healthspan of a subject.

The present disclosure also provides a method of increasing the healthspan of a subject, comprising administering a therapeutically or prophylactically effective amount of an agent capable of inhibiting interleukin 11 (IL-11)-mediated signalling to the subject.

In some embodiments, the agent is selected from the group consisting of: an antibody or an antigen-binding fragment thereof, a polypeptide, a peptide, a nucleic acid, an oligonucleotide, an aptamer or a small molecule.

In some embodiments, the agent is an agent capable of preventing or reducing the binding of interleukin 11 (IL-11) to a receptor for interleukin 11 (IL-11R).

In some embodiments, the agent is capable of binding to interleukin 11 (IL-11) or a receptor for interleukin 11 (IL-11R).

In some embodiments, the agent is an antibody or an antigen-binding fragment thereof.

In some embodiments, the agent is an anti-IL-11 antibody antagonist of IL-11-mediated signalling, or an antigen-binding fragment thereof.

In some embodiments, the agent is an anti-IL-11Rα antibody antagonist of IL-11-mediated signalling, or an antigen-binding fragment thereof.

In some embodiments, the agent is a decoy receptor for IL-11.

In some embodiments, the agent is a competitive inhibitor of IL-11.

In some embodiments, the agent is capable of preventing or reducing the expression of interleukin 11 (IL-11) or a receptor for interleukin 11 (IL-11R).

In some embodiments, the agent is an antisense oligonucleotide capable of preventing or reducing the expression of IL-11.

In some embodiments, the agent is an antisense oligonucleotide capable of preventing or reducing the expression of IL-11Rα.

In some embodiments, the method comprises administering the agent to a subject in which expression of interleukin 11 (IL-11) or a receptor for IL-11 (IL-11R) is upregulated.

### DESCRIPTION

### Interleukin 11 and Receptors for IL-11

Interleukin 11 (IL-11), also known as adipogenesis inhibitory factor, is a pleiotropic cytokine and a member of the IL-6 family of cytokines that includes IL-6, IL-11, IL-27, IL-31, oncostatin, leukemia inhibitory factor (LIF), cardiotrophin-1 (CT-1), cardiotrophin-like cytokine (CLC), ciliary neurotrophic factor (CNTF) and neuropoetin (NP-1).

Interleukin 11 (IL-11) is expressed in a variety of cell types. IL-11 genomic sequences have been mapped onto chromosome 19 and the centromeric region of chromosome 7, and is transcribed with a canonical signal peptide that ensures efficient secretion from cells. The activator protein complex of IL-11, cJun/AP-1, located within its promoter sequence is critical for basal transcriptional regulation of IL-11 (Du and Williams., Blood 1997, Vol 89: 3897-3908). The immature form of human IL-11 is a 199 amino acid polypeptide whereas the mature form of IL-11 encodes a protein of 178 amino acid residues (Garbers and Scheller., Biol. Chem. 2013; 394(9):1145-1161). The human IL-11 amino acid sequence is available under UniProt accession no. P20809 (P20809.1 GI:124294; SEQ ID NO:1). Recombinant human IL-11 (oprelvekin) is also commercially available. IL-11 from other species, including mouse, rat, pig, cow, several species of bony fish and primates, have also been cloned and sequenced.

In this specification “IL-11” refers to an IL-11 from any species and includes isoforms, fragments, variants or homologues of an IL-11 from any species. In preferred embodiments the species is human (Homo sapiens). Isoforms, fragments, variants or homologues of an IL-11 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature IL-11 from a given species, e.g. human. Isoforms, fragments, variants or homologues of an IL-11 may optionally be characterised by ability to bind IL-11Rα (preferably from the same species) and stimulate signal transduction in cells expressing IL-11Rα and gp130 (e.g. as described in Curtis et al. Blood, 1997, 90(11); or Karpovich et al. Mol. Hum. Reprod. 2003 9(2): 75-80). A fragment of IL-11 may be of any length (by number of amino acids), although may optionally be at least 25% of the length of mature IL-11 and may have a maximum length of one of 50%, 75%, 80%, 85%, 90%, 91%, 92%, 93%, 94%, 95%, 96%, 97%, 98%, or 99% of the length of mature IL-11. A fragment of IL-11 may have a minimum length of 10 amino acids, and a maximum length of one of 15, 20, 25, 30, 40, 50, 60, 70, 80, 90, 100, 110, 120, 130, 140, 150, 160, 170, 180, 190 or 195 amino acids.

IL-11 signals through a homodimer of the ubiquitously expressed glycoprotein 130 (gp130; also known as glycoprotein 130, IL-6ST, IL-6-beta or CD130). Gp130 is a transmembrane protein that forms one subunit of the type I cytokine receptor with the IL-6 receptor family. Specificity is gained through an individual interleukin 11 receptor subunit alpha (IL-11Rα), which does not directly participate in signal transduction, although the initial cytokine binding event to the α-receptor leads to the final complex formation with gp130.

Human gp130 (including the 22 amino acid signal peptide) is a 918 amino acid protein, and the mature form is 866 amino acids, comprising a 597 amino acid extracellular domain, a 22 amino acid transmembrane domain, and a 277 amino acid intracellular domain. The extracellular domain of the protein comprises the cytokine-binding module (CBM) of gp130. The CBM of gp130 comprises the Ig-like domain D1, and the fibronectin-type III domains D2 and D3 of gp130. The amino acid sequence of human gp130 is available under UniProt accession no. P40189-1 (SEQ ID NO:2).

Human IL-11Rα is a 422 amino acid polypeptide (UniProt Q14626; SEQ ID NO:3) and shares ˜85% nucleotide and amino acid sequence identity with the murine IL-11Rα. Two isoforms of IL-11Rα have been reported, which differ in the cytoplasmic domain (Du and Williams, supra). The IL-11 receptor α-chain (IL-11Rα) shares many structural and functional similarities with the IL-6 receptor α-chain (IL-6Rα). The extracellular domain shows 24% amino acid identity including the characteristic conserved Trp-Ser-X-Trp-Ser (WSXWS) motif. The short cytoplasmic domain (34 amino acids) lacks the Box 1 and 2 regions that are required for activation of the JAK/STAT signalling pathway.

The receptor binding sites on murine IL-11 have been mapped and three sites—sites 1, 11 and III—identified. Binding to gp130 is reduced by substitutions in the site II region and by substitutions in the site III region. Site III mutants show no detectable agonist activity and have IL-11Rα antagonist activity (Cytokine Inhibitors Chapter 8; edited by Gennaro Ciliberto and Rocco Savino, Marcel Dekker, Inc. 2001).

In this specification a receptor for IL-11 (IL-11R) refers to a polypeptide or polypeptide complex capable of binding IL-11. In some embodiments an IL-11 receptor is capable of binding IL-11 and inducing signal transduction in cells expressing the receptor.

An IL-11 receptor may be from any species and includes isoforms, fragments, variants or homologues of an IL-11 receptor from any species. In preferred embodiments the species is human (Homo sapiens).

In some embodiments the IL-11 receptor may be IL-11Rα. In some embodiments a receptor for IL-11 may be a polypeptide complex comprising IL-11Rα. In some embodiments the IL-11 receptor may be a polypeptide complex comprising IL-11Rα and gp130. In some embodiments the IL-11 receptor may be gp130 or a complex comprising gp130 to which IL-11 binds.

Isoforms, fragments, variants or homologues of an IL-11Rα may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of IL-11Rα from a given species, e.g. human. Isoforms, fragments, variants or homologues of an IL-11Rα may optionally be characterised by ability to bind IL-11 (preferably from the same species) and stimulate signal transduction in cells expressing the IL-11Rα and gp130 (e.g. as described in Curtis et al. Blood, 1997, 90(11) or Karpovich et al. Mol. Hum. Reprod. 2003 9(2): 75-80). A fragment of an IL-11 receptor may be of any length (by number of amino acids), although may optionally be at least 25% of the length of the mature IL-11Rα and have a maximum length of one of 50%, 75%, 80%, 85%, 90%, 91%, 92%, 93%, 94%, 95%, 96%, 97%, 98%, or 99% of the length of the mature IL-11Rα. A fragment of an IL-11 receptor fragment may have a minimum length of 10 amino acids, and a maximum length of one of 15, 20, 25, 30, 40, 50, 100, 110, 120, 130, 140, 150, 160, 170, 180, 190, 200, 250, 300, 400, or 415 amino acids.

IL-11 signalling

IL-11 binds to IL-11Rα with low affinity (Kd˜22 nM; see Metcalfe et al., JBC (2020) Manuscript RA119.012351), and interaction between these binding partners alone is insufficient to transduce a biological signal. The generation of a high affinity receptor (Kd˜400 to 800 pmol/L) capable of signal transduction requires co-expression of the IL-11Rα and gp130 (Curtis et al Blood 1997; 90 (11):4403-12; Hilton et al., EMBO J 13:4765, 1994; Nandurkar et al., Oncogene 12:585, 1996). Binding of IL-11 to cell-surface IL-11Rα induces heterodimerization, tyrosine phosphorylation, activation of gp130 and downstream signalling, predominantly through the mitogen-activated protein kinase (MAPK)-cascade and the Janus kinase/signal transducer and activator of transcription (Jak/STAT) pathway (Garbers and Scheller, supra).

In principle, a soluble IL-11Rα can also form biologically active soluble complexes with IL-11 (Pflanz et al., 1999 FEBS Lett, 450, 117-122) raising the possibility that, similar to IL-6, IL-11 may in some instances bind soluble IL-11Rα prior to binding cell-surface gp130 (Garbers and Scheller, supra). Curtis et al (Blood 1997 Dec. 1; 90 (11):4403-12) describe expression of a soluble murine IL-11 receptor alpha chain (sIL-11R) and examined signalling in cells expressing gp130. In the presence of gp130 but not transmembrane IL-11R the sIL-11R mediated IL-11 dependent differentiation of M1 leukemic cells and proliferation in Ba/F3 cells and early intracellular events including phosphorylation of gp130, STAT3 and SHP2 similar to signalling through transmembrane IL-11R. Activation of signalling through cell-membrane bound gp130 by IL-11 bound to soluble IL-11Rα has recently been demonstrated (Lokau et al., 2016 Cell Reports 14, 1761-1773). This so-called IL-11 trans signalling may be important for disease pathogenesis, yet its role in human disease has not yet been studied.

As used herein, ‘IL-11 trans signalling’ is used to refer to signalling which is triggered by binding of IL-11 bound to IL-11Rα, to gp130. The IL-11 may be bound to IL-11Rα as a non-covalent complex. The gp130 is membrane-bound and expressed by the cell in which signalling occurs following binding of the IL-11:IL-11Rα complex to gp130. In some embodiments the IL-11Rα may be a soluble IL-11Rα. In some embodiments, the soluble IL-11Rα is a soluble (secreted) isoform of IL-11Rα (e.g. lacking a transmembrane domain). In some embodiments, the soluble IL-11Rα is the liberated product of proteolytic cleavage of the extracellular domain of cell membrane bound IL-11Rα. In some embodiments, the IL-11Rα may be cell membrane-bound, and signalling through gp130 may be triggered by binding of IL-11 bound to cell-membrane-bound IL-11Rα, termed “IL-11 cis signalling”. In preferred embodiments, inhibition of IL-11-mediated signalling is achieved by disrupting IL-11-mediated cis signalling.

IL-11-mediated signalling has been shown to stimulate hematopoiesis and thrombopoiesis, stimulate osteoclast activity, stimulate neurogenesis, inhibit adipogenesis, reduce pro inflammatory cytokine expression, modulate extracellular matrix (ECM) metabolism, and mediate normal growth control of gastrointestinal epithelial cells (Du and Williams, supra).

The physiological role of Interleukin 11 (IL-11) remains unclear. IL-11 has been most strongly linked with activation of haematopoetic cells and with platelet production. IL-11 has also been shown to confer protection against graft-vs-host-disease, inflammatory arthritis and inflammatory bowel disease, leading to IL-11 being considered an anti-inflammatory cytokine (Putoczki and Ernst, J Leukoc Biol 2010, 88(6):1109-1117). However, it is suggested that IL-11 is pro-inflammatory as well as anti-inflammatory, pro-angiogenic and important for neoplasia. Recent studies have shown that IL-11 is readily detectable during viral-induced inflammation in a mouse arthritis model and in cancers, suggesting that the expression of IL-11 can be induced by pathological stimuli. IL-11 is also linked to Stat3-dependent activation of tumour-promoting target genes in neoplastic gastrointestinal epithelium (Putoczki and Ernst, supra).

As used herein, “IL-11 signalling” and “IL-11-mediated signalling” refers to signalling mediated by binding of IL-11, or a fragment thereof having the function of the mature IL-11 molecule, to a receptor for IL-11. It will be appreciated that “IL-11 signalling” and “IL-11 mediated signalling” refer to signalling initiated by IL-11/functional fragment thereof, e.g. through binding to a receptor for IL-11. “Signalling” in turn refers to signal transduction and other cellular processes governing cellular activity.

### Agents Capable of Inhibiting the Action of IL-11

Aspects of the present disclosure involve inhibition of IL-11-mediated signalling.

Herein, ‘inhibition’ refers to a reduction, decrease or lessening relative to a control condition. For example, inhibition of the action of IL-11 by an agent capable of inhibiting IL-11-mediated signalling refers to a reduction, decrease or lessening of the extent/degree of IL-11-mediated signalling in the absence of the agent, and/or in the presence of an appropriate control agent.

Inhibition may herein also be referred to as neutralisation or antagonism. That is, an agent capable of inhibiting IL-11-mediated signalling (e.g. interaction, signalling or other activity mediated by IL-11 or an IL-11-containing complex) may be said to be a ‘neutralising’ or ‘antagonist’ agent with respect to the relevant function or process. For example, an agent which is capable of inhibiting IL-11-mediated signalling may be referred to as an agent which is capable of neutralising IL-11-mediated signalling, or may be referred to as an antagonist of IL-11-mediated signalling.

The IL-11 signalling pathway offers multiple routes for inhibition of IL-11 signalling. An agent capable of inhibiting IL-11-mediated signalling may do so e.g. through inhibiting the action of one or more factors involved in, or necessary for, signalling through a receptor for IL-11.

For example, inhibition of IL-11 signalling may be achieved by disrupting interaction between IL-11 (or an IL-11 containing complex, e.g. a complex of IL-11 and IL-11Rα) and a receptor for IL-11 (e.g. IL-11Rα, a receptor complex comprising IL-11Rα, gp130 or a receptor complex comprising IL-11Rα and gp130). In some embodiments, inhibition of IL-11-mediated signalling is achieved by inhibiting the gene or protein expression of one or more of e.g. IL-11, IL-11Rα and gp130.

Inhibition of IL-11-mediated signalling may also be achieved by disrupting interaction between IL-11:11 receptor complexes (i.e. complexes comprising IL-11 and IL-11Rα, or IL-11 and gp130, or IL-11, IL-11Rα and gp130) to form multimers (e.g. hexameric complexes) required for activation of downstream signalling by cells expressing IL-11 receptors.

In embodiments, inhibition of IL-11-mediated signalling is achieved by disrupting IL-11-mediated cis signalling but not disrupting IL-11-mediated trans signalling, e.g. inhibition of IL-11-mediated signalling is achieved by inhibiting gp130-mediated cis complexes involving membrane bound IL-11Rα. In embodiments, inhibition of IL-11-mediated signalling is achieved by disrupting IL-11-mediated trans signalling but not disrupting IL-11-mediated cis signalling, i.e. inhibition of IL-11-mediated signalling is achieved by inhibiting gp130-mediated trans signalling complexes such as IL-11 bound to soluble IL-11Rα or IL-6 bound to soluble IL-6R. In embodiments, inhibition of IL-11-mediated signalling is achieved by disrupting IL-11-mediated cis signalling and IL-11-mediated trans signalling. Any agent as described herein may be used to inhibit IL-11-mediated cis and/or trans signalling.

In other examples, inhibition of IL-11 signalling may be achieved by disrupting signalling pathways downstream of IL-11/IL-11Rα/gp130. That is, in some embodiments inhibition/antagonism of IL-11-mediated signalling comprises inhibition of a signalling pathway/process/factor downstream of signalling through the IL-11/IL-11 receptor complex.

In some embodiments inhibition/antagonism of IL-11-mediated signalling comprises inhibition of signalling through an intracellular signalling pathway which is activated by the IL-11/IL-11 receptor complex. In some embodiments inhibition/antagonism of IL-11-mediated signalling comprises inhibition of one or more factors whose expression/activity is upregulated as a consequence of signalling through the IL-11/IL-11 receptor complex.

In some embodiments, the methods of the present disclosure employ agents capable of inhibiting JAK/STAT signalling. In some embodiments, agents capable of inhibiting JAK/STAT signalling are capable of inhibiting the action of JAK1, JAK2, JAK3, TYK2, STAT1, STAT2, STAT3, STAT4, STAT5A, STAT5B and/or STAT6. For example, agents may be capable of inhibiting activation of JAK/STAT proteins, inhibiting interaction of JAK or STAT proteins with cell surface receptors e.g. IL-11Rα or gp130, inhibiting phosphorylation of JAK proteins, inhibiting interaction between JAK and STAT proteins, inhibiting phosphorylation of STAT proteins, inhibiting dimerization of STAT proteins, inhibiting translocation of STAT proteins to the cell nucleus, inhibiting binding of STAT proteins to DNA, and/or promoting degradation of JAK and/or STAT proteins. In some embodiments, a JAK/STAT inhibitor is selected from Ruxolitinib (Jakafi/Jakavi; Incyte), Tofacitinib (Xeljanz/Jakvinus; NIH/Pfizer), Oclacitinib (Apoquel), Baricitinib (Olumiant; Incyte/Eli Lilly), Filqotinib (G-146034/GLPG-0634; Galapagos NV), Gandotinib (LY-2784544; Eli Lilly), Lestaurtinib (CEP-701; Teva), Momelotinib (GS-0387/CYT-387; Gilead Sciences), Pacritinib (SB1518; CTI), PF-04965842 (Pfizer), Upadacitinib (ABT-494; AbbVie), Peficitinib (ASP015K/JNJ-54781532; Astellas), Fedratinib (SAR302503; Celgene), Cucurbitacin I (JSI-124) and CHZ868.

In some embodiments, the methods of the present disclosure employ agents capable of inhibiting MAPK/ERK signalling. In some embodiments, agents capable of inhibiting MAPK/ERK signalling are capable of inhibiting the action of GRB2, inhibiting the action of RAF kinase, inhibiting the action of MEK proteins, inhibiting the activation of MAP3K/MAP2K/MAPK and/or Myc, and/or inhibiting the phosphorylation of STAT proteins. In some embodiments, agents capable of inhibiting ERK signalling are capable of inhibiting ERK p42/44. In some embodiments, an ERK inhibitor is selected from SCH772984, SC1, VX-11e, DEL-22379, Sorafenib (Nexavar; Bayer/Onyx), SB590885, PLX4720, XL281, RAF265 (Novartis), encorafenib (LGX818/Braftovi; Array BioPharma), dabrafenib (Tafinlar; GSK), vemurafenib (Zelboraf; Roche), cobimetinib (Cotellic; Roche), CI-1040, PD0325901, Binimetinib (MEK162/MEKTOVI; Array BioPharma), selumetinib (AZD6244; Array/AstraZeneca) and Trametinib (GSK1120212/Mekinist; Novartis). In some embodiments, the methods of the present disclosure employ agents capable of inhibiting c-Jun N-terminal kinase (JNK) signalling/activity. In some embodiments, agents capable of inhibiting JNK signalling/activity are capable of inhibiting the action and/or phosphorylation of a JNK (e.g. JNK1, JNK2). In some embodiments, a JNK inhibitor is selected from SP600125, CEP 1347, TCS JNK 60, c-JUN peptide, SU3327, AEG 3482, TCS JNK 5a, BI78D3, IQ3, SR3576, IQ1S, JIP-1 (153-163) and CC401 dihydrochloride.

In the present Examples the inventors demonstrate that NOX4 expression and activity is upregulated by signalling through IL-11/IL-11Rα/gp130. NOX4 is an NADPH oxidase, and a source of reactive oxygen species (ROS). Expression of Nox4 is upregulated in transgenic mice with hepatocyte-specific Ill 1 expression, and primary human hepatocytes stimulated with IL11 upregulate NOX4 expression.

In some embodiments, the present disclosure employs agents capable of inhibiting NOX4 expression (gene or protein expression) or function. In some embodiments, the present disclosure employs agents capable of inhibiting IL-11-mediated upregulation of NOX4 expression/function. Agents capable of inhibiting NOX4 expression or function may be referred to herein as NOX4 inhibitors. For example, a NOX4 inhibitor may be capable of reducing expression (e.g. gene and/or protein expression) of NOX4, reducing the level of RNA encoding NOX4, reduce the level of NOX4 protein, and/or reducing the level of a NOX4 activity (e.g. reducing NOX4-mediated NADPH oxidase activity and/or NOX4-mediated ROS production).

NOX4 inhibitors include a NOX4-binding molecules and molecules capable of reducing NOX4 expression. NOX4-binding inhibitors include peptide/nucleic acid aptamers, antibodies (and antibody fragments) and fragments of interaction partners for NOX4 which behave as antagonists of NOX4 function, and small molecules inhibitors of NOX4. Molecules capable of reducing NOX4 expression include antisense RNA (e.g. siRNA, shRNA) to NOX4. In some embodiments, a NOX4 inhibitor is selected from a NOX4 inhibitor described in Altenhofer et al., Antioxid Redox Signal. (2015) 23(5): 406-427 or Augsburder et al., Redox Biol. (2019) 26: 101272, such as GKT137831.

### Binding Agents

In some embodiments, agents capable of inhibiting IL-11-mediated signalling may bind to IL-11. In some embodiments, agents capable of inhibiting IL-11-mediated signalling may bind to a receptor for IL-11 (e.g. IL-11Rα, gp130, or a complex containing IL-11Rα and/or gp130). Binding of such agents may inhibit IL-11-mediated signalling by reducing/preventing the ability of IL-11 to bind to receptors for IL-11, thereby inhibiting downstream signalling. Binding of such agents may inhibit IL-11 mediated cis and/or trans-signalling by reducing/preventing the ability of IL-11 to bind to receptors for IL-11, e.g. IL-11Rα and/or gp130, thereby inhibiting downstream signalling. Agents may bind to trans-signalling complexes such as IL-11 and soluble IL-11Rα and inhibit gp130-mediated signalling.

Agents capable of binding to IL-11/an IL-11 containing complex or a receptor for IL-11 may be of any kind, but in some embodiments the agent may be an antibody, an antigen-binding fragment thereof, a polypeptide, a peptide, a nucleic acid, an oligonucleotide, an aptamer or a small molecule. The agents may be provided in isolated or purified form, or may be formulated as a pharmaceutical composition or medicament.

### Antibodies and Antigen-Binding Fragments

In some embodiments, an agent capable of binding to IL-11/an IL-11 containing complex or a receptor for IL-11 is an antibody, or an antigen-binding fragment thereof. In some embodiments, an agent capable of binding to IL-11/an IL-11 containing complex or a receptor for IL-11 is a polypeptide, e.g. a decoy receptor molecule. In some embodiments, an agent capable of binding to IL-11/an IL-11 containing complex or a receptor for IL-11 may be an aptamer.

In some embodiments, an agent capable of binding to IL-11/an IL-11 containing complex or a receptor for IL-11 is an antibody, or an antigen-binding fragment thereof. An “antibody” is used herein in the broadest sense, and encompasses monoclonal antibodies, polyclonal antibodies, monospecific and multispecific antibodies (e.g., bispecific antibodies), and antibody fragments, as long as they display binding to the relevant target molecule.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Monoclonal antibodies (mAbs) are particularly useful in the methods of the present disclosure, and are a homogenous population of antibodies specifically targeting a single epitope on an antigen.

Polyclonal antibodies are also useful in the methods of the present disclosure. Monospecific polyclonal antibodies are preferred. Suitable polyclonal antibodies can be prepared using methods well-known in the art.

Antigen-binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Nat. Acad. Sd. USA 81, 6851-6855).

Antibodies and antigen-binding fragments according to the present disclosure comprise the complementarity-determining regions (CDRs) of an antibody which is capable of binding to the relevant target molecule (i.e. IL-11/an IL-11 containing complex/a receptor for IL-11).

In some embodiments, the agent is an IL-11-binding antibody, or an IL-11-binding fragment thereof. Antibodies which bind to IL-11 include e.g. monoclonal mouse anti-human IL-11 antibody clone #22626; Catalog No. MAB218 (R&D Systems, MN, USA), used e.g. in Bockhorn et al. Nat. Commun. (2013) 4(0):1393, clone 6D9A (Abbiotec), clone KT8 (Abbiotec), clone M3103F11 (BioLegend), clone 1F1 (Abnova Corporation), clone 3C6 (Abnova Corporation), clone GF1 (LifeSpan Biosciences), clone 13455 (Source BioScience), 11h3/19.6.1 (Hermann et al., Arthritis Rheum. (1998) 41(8):1388-97), AB-218-NA (R&D Systems), X203 (Ng et al., Sci Transl Med. (2019) 11(511) pii: eaaw1237) and anti-IL-11 antibodies disclosed in US 2009/0202533 A1, WO 99/59608 A2, WO 2018/109174 A2 and WO 2019/238882 A1.

In particular, anti-IL-11 antibody clone 22626 (also known as MAB218) has been shown to be an antagonist of IL-11 mediated signalling, e.g. in Schaefer et al., Nature (2017) 552(7683):110-115. Monoclonal antibody 11 h3/19.6.1 is disclosed in Hermann et al., Arthritis Rheum. (1998) 41(8):1388-97 to be a neutralising anti-IL-11 IgG1. AB-218-NA from R&D Systems, used e.g. in McCoy et al., BMC Cancer (2013) 13:16, is another example of neutralizing anti-IL-11 antibody. WO 2018/109174 A2 and WO 2019/238882 A1 disclose yet further exemplary anti-IL-11 antibody antagonists of IL-11 mediated signalling. X203 (also referred to as Enx203) disclosed in Ng, et al., “IL-11 is a therapeutic target in idiopathic pulmonary fibrosis.” bioRxiv 336537; doi: https://doi.org/10.1101/336537 and WO 2019/238882 A1 is an anti-IL-11 antibody antagonist of IL-11-mediated signalling, and comprises the VH region according to SEQ ID NO:92 of WO 2019/238882 A1 (SEQ ID NO:22 of the present disclosure), and the VL region according to SEQ ID NO:94 of WO 2019/238882 A1 (SEQ ID NO:23 of the present disclosure). Humanised versions of the X203 are described in WO 2019/238882 A1, including hEnx203 which comprises the VH region according to SEQ ID NO:117 of WO 2019/238882 A1 (SEQ ID NO:30 of the present disclosure), and the VL region according to SEQ ID NO:122 of WO 2019/238882 A1 (SEQ ID NO:31 of the present disclosure). Enx108A is a further example of an anti-IL-11 antibody antagonist of IL-11-mediated signalling, and comprises the VH region according to SEQ ID NO:8 of WO 2019/238882 A1 (SEQ ID NO:26 of the present disclosure), and the VL region according to SEQ ID NO:20 of WO 2019/238882 A1 (SEQ ID NO:27 of the present disclosure).

In some embodiments, the agent is an IL-11Rα-binding antibody, or an IL-11Rα-binding fragment thereof. Antibodies which bind to IL-11Rα include e.g. monoclonal antibody clone 025 (Sino Biological), clone EPR5446 (Abcam), clone 473143 (R & D Systems), clones 8E2, 8D10 and 8E4 and the affinity-matured variants of 8E2 described in US 2014/0219919 A1, the monoclonal antibodies described in Blanc et al (J. Immunol Methods. 2000 Jul. 31; 241(1-2); 43-59), X209 (Widjaja et al., Gastroenterology (2019) 157(3):777-792, which is also published as Widjaja, et al., “IL-11 neutralising therapies target hepatic stellate cell-induced liver inflammation and fibrosis in NASH.” bioRxiv 470062; doi: https://doi.org/10.1101/470062) antibodies disclosed in WO 2014121325 A1 and US 2013/0302277 A1, and anti-IL-11Rα antibodies disclosed in US 2009/0202533 A1, WO 99/59608 A2, WO 2018/109170 A2 and WO 2019/238884 A1.

In particular, anti-IL-11Rα antibody clone 473143 (also known as MAB1977) has been shown to be an antagonist of IL-11 mediated signalling, e.g. in Schaefer et al., Nature (2017) 552(7683):110-115. US 2014/0219919 A1 provides sequences for anti-human IL-11Rα antibody clones 8E2, 8D10 and 8E4, and discloses their ability to antagonise IL-11 mediated signalling—see e.g. [0489] to [0490] of US 2014/0219919 A1. US 2014/0219919 A1 moreover provides sequence information for an additional 62 affinity-matured variants of clone 8E2, 61 of which are disclosed to antagonise IL-11 mediated signalling—see Table 3 of US 2014/0219919 A1. WO 2018/109170 A2 and WO 2019/238884 A1 disclose yet further exemplary anti-IL-11Rα antibody antagonists of IL-11 mediated signalling. X209 (also referred to as Enx209) disclosed in Widjaja, et al., “IL-11 neutralising therapies target hepatic stellate cell-induced liver inflammation and fibrosis in NASH.” bioRxiv 470062; doi: https://doi.org/10.1101/470062 and WO 2019/238884 A1 is an anti-IL-11Rα antibody antagonist of IL-11-mediated signalling, and comprises the VH region according to SEQ ID NO:7 of WO 2019/238884 A1 (SEQ ID NO:24 of the present disclosure), and the VL region according to SEQ ID NO:14 of WO 2019/238884 A1 (SEQ ID NO:25 of the present disclosure). Humanised versions of the X209 are described in WO 2019/238884 A1, including hEnx209 which comprises the VH region according to SEQ ID NO:11 of WO 2019/238884 A1 (SEQ ID NO:32 of the present disclosure), and the VL region according to SEQ ID NO:17 of WO 2019/238884 A1 (SEQ ID NO:33 of the present disclosure).

The skilled person is well aware of techniques for producing antibodies suitable for therapeutic use in a given species/subject. For example, procedures for producing antibodies suitable for therapeutic use in humans are described in Park and Smolen Advances in Protein Chemistry (2001) 56: 369-421 (hereby incorporated by reference in its entirety).

Antibodies to a given target protein (e.g. IL-11 or IL-11Rα) can be raised in model species (e.g. rodents, lagomorphs), and subsequently engineered in order to improve their suitability for therapeutic use in a given species/subject. For example, one or more amino acids of monoclonal antibodies raised by immunisation of model species can be substituted to arrive at an antibody sequence which is more similar to human germline immunoglobulin sequences (thereby reducing the potential for anti-xenogenic antibody immune responses in the human subject treated with the antibody). Modifications in the antibody variable domains may focus on the framework regions in order to preserve the antibody paratope. Antibody humanisation is a matter of routine practice in the art of antibody technology, and is reviewed e.g. in Almagro and Fransson, Frontiers in Bioscience (2008) 13:1619-1633, Safdari et al., Biotechnology and Genetic Engineering Reviews (2013) 29(2): 175-186 and Lo et al., Microbiology Spectrum (2014) 2(1), all of which are hereby incorporated by reference in their entirety. The requirement for humanisation can be circumvented by raising antibodies to a given target protein (e.g. IL-11 or IL-11Rα) in transgenic model species expressing human immunoglobulin genes, such that the antibodies raised in such animals are fully-human (described e.g. in Bruggemann et al., Arch Immunol Ther Exp (Warsz) (2015) 63(2):101-108, which is hereby incorporated by reference in its entirety).

Phage display techniques may also be employed to the identification of antibodies to a given target protein (e.g. IL-11 or IL-11Rα), and are well-known to the skilled person. The use of phage display for the identification of fully human antibodies to human target proteins is reviewed e.g. in Hoogenboom, Nat. Biotechnol. (2005) 23, 1105-1116 and Chan et al., International Immunology (2014) 26(12): 649-657, which are hereby incorporated by reference in their entirety.

The antibodies/fragments may be antagonist antibodies/fragments that inhibit or reduce a biological activity of IL-11. The antibodies/fragments may be neutralising antibodies that neutralise the biological effect of IL-11, e.g. its ability to stimulate productive signalling via an IL-11 receptor. Neutralising activity may be measured by ability to neutralise IL-11 induced proliferation in the T11 mouse plasmacytoma cell line (Nordan, R. P. et al. (1987) J. Immunol. 139:813).

IL-11- or IL-11Rα-binding antibodies can be evaluated for the ability to antagonise IL-11-mediated signalling, e.g. using the assay described in US 2014/0219919 A1 or Blanc et al (J. Immunol Methods. 2000 Jul. 31; 241(1-2); 43-59. Briefly, IL-11- and IL-11Rα-binding antibodies can be evaluated in vitro for the ability to inhibit proliferation of Ba/F3 cells expressing IL-11Rα and gp130 from the appropriate species, in response to stimulation with IL-11 from the appropriate species. Alternatively, IL-11- and IL-11Rα-binding antibodies can be analysed in vitro for the ability to inhibit the fibroblast-to-myofibroblast transition following stimulation of fibroblasts with TGFβ1, by evaluation of αSMA expression (as described e.g. in WO 2018/109174 A2 (Example 6) and WO 2018/109170 A2 (Example 6), Ng et al., Sci Transl Med. (2019) 11(511) pii: eaaw1237 and Widjaja et al., Gastroenterology (2019) 157(3):777-792).

Antibodies generally comprise six CDRs; three in the light chain variable region (VL): LC-CDR1, LC-CDR2, LC-CDR3, and three in the heavy chain variable region (VH): HC-CDR1, HC-CDR2 and HC-CDR3. The six CDRs together define the paratope of the antibody, which is the part of the antibody which binds to the target molecule. The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibodies described herein are defined according to the Kabat system.

In some embodiments an antibody, or an antigen-binding fragment thereof, according to the present disclosure is derived from an antibody which binds specifically to IL-11 (e.g. Enx108A, Enx203 or hEnx203). In some embodiments an antibody, or an antigen-binding fragment thereof, according to the present disclosure is derived from an antibody which binds specifically to IL-11Rα (e.g. Enx209 or hEnx209).

Antibodies and antigen-binding fragments according to the present disclosure preferably inhibit IL-11-mediated signalling. Such antibodies/antigen-binding fragments may be described as being antagonists of IL-11-mediated signalling, and/or may be described as having the ability to neutralise IL-11-mediated signalling.

In some embodiments, the antibody/antigen-binding fragment comprises the CDRs of an antibody which binds to IL-11. In some embodiments the antibody/antigen-binding fragment comprises the CDRs of, or CDRs derived from, the CDRs of an IL-11-binding antibody described herein (e.g. Enx108A, Enx203 or hEnx203).

In some embodiments the antibody/antigen-binding fragment comprises a VH region incorporating the following CDRs:
(1)
   HC-CDR1 having the amino acid sequence of SEQ ID NO:34
   HC-CDR2 having the amino acid sequence of SEQ ID NO:35
   HC-CDR3 having the amino acid sequence of SEQ ID NO:36,
   or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:
(2)
   LC-CDR1 having the amino acid sequence of SEQ ID NO:37
   LC-CDR2 having the amino acid sequence of SEQ ID NO:38
   LC-CDR3 having the amino acid sequence of SEQ ID NO:39,
   or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the following CDRs:
(3)
   HC-CDR1 having the amino acid sequence of SEQ ID NO:40
   HC-CDR2 having the amino acid sequence of SEQ ID NO:41
   HC-CDR3 having the amino acid sequence of SEQ ID NO:42,
   or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:
(4)
   LC-CDR1 having the amino acid sequence of SEQ ID NO:43
   LC-CDR2 having the amino acid sequence of SEQ ID NO:44
   LC-CDR3 having the amino acid sequence of SEQ ID NO:45,
   or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (1), and a VL region incorporating the CDRs according to (2). In some embodiments the antibody/antigen-binding fragment comprises a VH region incorporating the CDRs according to (3), and a VL region incorporating the CDRs according to (4).

In some embodiments the antibody/antigen-binding fragment comprises the VH region and the VL region of an antibody which binds to IL-11. In some embodiments the antibody/antigen-binding fragment comprises the VH region and VL region of, or a VH region and VL region derived from, the VH region and VL region of an IL-11-binding antibody described herein (e.g. Enx108A, Enx203 or hEnx203).

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31.

In some embodiments, the antibody/antigen-binding fragment comprises the CDRs of an antibody which binds to IL-11Rα. In some embodiments the antibody/antigen-binding fragment comprises the CDRs of, or CDRs derived from, the CDRs of an IL-11Rα-binding antibody described herein (e.g. Enx209 or hEnx209).

In some embodiments the antibody/antigen-binding fragment comprises a VH region incorporating the following CDRs:
(5)
   HC-CDR1 having the amino acid sequence of SEQ ID NO:46
   HC-CDR2 having the amino acid sequence of SEQ ID NO:47
   HC-CDR3 having the amino acid sequence of SEQ ID NO:48,
   or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:
(6)
   LC-CDR1 having the amino acid sequence of SEQ ID NO:49
   LC-CDR2 having the amino acid sequence of SEQ ID NO:50
   LC-CDR3 having the amino acid sequence of SEQ ID NO:51,
   or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (5), and a VL region incorporating the CDRs according to (6).

In some embodiments the antibody/antigen-binding fragment comprises the VH region and the VL region of an antibody which binds to IL-11Rα. In some embodiments the antibody/antigen-binding fragment comprises the VH region and VL region of, or a VH region and VL region derived from, the VH region and VL region of an IL-11Rα-binding antibody described herein (e.g. Enx209 or hEnx209).

In embodiments in accordance with the present disclosure in which one or more amino acids of a reference amino acid sequence (e.g. a CDR sequence, VH region sequence or VL region sequence described herein) are substituted with another amino acid, the substitutions may conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | GAP |
| | | ILV |
| | Polar - uncharged | CSTM |
| | | NQ |
| | Polar - charged | DE |
| | | KR |
| AROMATIC | | HFWY |

In some embodiments, substitution(s) may be functionally conservative. That is, in some embodiments the substitution may not affect (or may not substantially affect) one or more functional properties (e.g. target binding) of the antibody/fragment comprising the substitution relative to the equivalent unsubstituted molecule.

In some embodiments, substitution(s) relative to a reference VH region or VL region sequence may be focussed in a particular region or regions of the VH region or VL region sequence. For example, variation from a reference VH region or VL region sequence may be focussed in one or more of the framework regions (FR1, FR2, FR3 and/or FR4).

Antibodies and antigen-binding fragments according to the present disclosure may be designed and prepared using the sequences of monoclonal antibodies (mAbs) capable of binding to the relevant target molecule. Antigen-binding regions of antibodies, such as single chain variable fragment (scFv), Fab and Fab2 fragments may also be used/provided. An ‘antigen-binding region’ or ‘antigen binding fragment’ is any fragment of an antibody which is capable of binding to the target for which the given antibody is specific.

In some embodiments the antibodies/fragments comprise the VL and VH regions of an antibody which is capable of binding to IL-11, an IL-11 containing complex, or a receptor for IL-11. The VL and VH region of an antigen-binding region of an antibody together constitute the Fv region. In some embodiments the antibodies/fragments comprise or consist of the Fv region of an antibody which is capable of binding to IL-11, an IL-11 containing complex, or a receptor for IL-11. The Fv region may be expressed as a single chain wherein the VH and VL regions are covalently linked, e.g. by a flexible oligopeptide. Accordingly, antibodies/fragments may comprise or consist of an scFv comprising the VL and VH regions of an antibody which is capable of binding to IL-11, an IL-11 containing complex, or a receptor for IL-11.

The VL and light chain constant (CL) region, and the VH region and heavy chain constant 1 (CH1) region of an antigen-binding region of an antibody together constitute the Fab region. In some embodiments the antibodies/fragments comprise or consist of the Fab region of an antibody which is capable of binding to IL-11, an IL-11 containing complex, or a receptor for IL-11.

In some embodiments, antibodies/fragments comprise, or consist of, whole antibody capable of binding to IL-11, an IL-11 containing complex, or a receptor for IL-11. A “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety. Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprises a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments the antibody/antigen-binding fragment of the present disclosure comprises an immunoglobulin heavy chain constant sequence. In some embodiments, an immunoglobulin heavy chain constant sequence may be a human immunoglobulin heavy chain constant sequence. In some embodiments the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some the immunoglobulin heavy chain constant sequence is, or is derived from, the heavy chain constant sequence of a human IgG1 allotype (e.g. G1m1, G1m2, G1m3 or G1m17).

In some embodiments the immunoglobulin heavy chain constant sequence is, or is derived from, the constant region sequence of human immunoglobulin G 1 constant (IGHG1; UniProt: P01857-1, v1). In some embodiments the immunoglobulin heavy chain constant sequence is, or is derived from, the constant region sequence of human immunoglobulin G 1 constant (IGHG1; UniProt: P01857-1, v1) comprising substitutions K214R, D356E and L358M (i.e. the G1m3 allotype). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.

In some embodiments the immunoglobulin heavy chain constant sequence is, or is derived from, the constant region sequence of human immunoglobulin G 4 constant (IGHG4; UniProt: P01861, v1). In some embodiments the immunoglobulin heavy chain constant sequence is, or is derived from, the constant region sequence of human immunoglobulin G 4 constant (IGHG4; UniProt: P01861, v1) comprising substitutions S241P and/or L248E. The S241P mutation is hinge stabilising while the L248E mutation further reduces the already low ADCC effector function of IgG4 (Davies and Sutton, Immunol Rev. 2015 November; 268(1):139-159; Angal et al Mol Immunol. 1993 January; 30(1):105-8). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.

In some embodiments the antibody/antigen-binding fragment of the present disclosure comprises an immunoglobulin light chain constant sequence. In some embodiments, an immunoglobulin light chain constant sequence may be a human immunoglobulin light chain constant sequence. In some embodiments the immunoglobulin light chain constant sequence is, or is derived from, a kappa (κ) or lambda (λ) light chain, e.g. human immunoglobulin kappa constant (IGKC; Cκ; UniProt: P01834-1, v2), or human immunoglobulin lambda constant (IGLC; Cλ), e.g. IGLC1 (UniProt: P0CG04-1, v1), IGLC2 (UniProt: P0DOY2-1, v1), IGLC3 (UniProt: P0DOY3-1, v1), IGLC6 (UniProt: P0CF74-1, v1) or IGLC7 (UniProt: A0M8Q6-1, v3).

In some embodiments the antibody/antigen-binding fragment comprises an immunoglobulin light chain constant sequence. In some embodiments the immunoglobulin light chain constant sequence is, or is derived from human immunoglobulin kappa constant (IGKC; Cκ; UniProt: P01834-1, v2; SEQ ID NO:90). In some embodiments the immunoglobulin light chain constant sequence is a human immunoglobulin lambda constant (IGLC; Cλ), e.g. IGLC1, IGLC2, IGLC3, IGLC6 or IGLC7. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:55.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:28, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:29.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:56, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:57.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:58, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:59.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies capable of binding to IL-11, an IL-11 containing complex, or a receptor for IL-11 may also be made using phage display technology as is well-known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies/fragments include bi-specific antibodies, e.g. composed of two different fragments of two different antibodies, such that the bi-specific antibody binds two types of antigen. The bispecific antibody comprises an antibody/fragment as described herein capable of binding to IL-11, an IL-11 containing complex, or a receptor for IL-11. The antibody may contain a different fragment having affinity for a second antigen, which may be any desired antigen. Techniques for the preparation of bi-specific antibodies are well-known in the art, e.g. see Mueller, D et al., (2010 Biodrugs 24 (2): 89-98), Wozniak-Knopp G et al., (2010 Protein Eng Des 23 (4): 289-297), and Baeuerle, P A et al., (2009 Cancer Res 69 (12): 4941-4944). Bispecific antibodies and bispecific antigen-binding fragments may be provided in any suitable format, such as those formats described in Kontermann MAbs 2012, 4(2): 182-197, which is hereby incorporated by reference in its entirety. For example, a bispecific antibody or bispecific antigen-binding fragment may be a bispecific antibody conjugate (e.g. an IgG2, F(ab′)2 or CovX-Body), a bispecific IgG or IgG-like molecule (e.g. an IgG, scFv4-Ig, IgG-scFv, scFv-IgG, DVD-Ig, IgG-sVD, sVD-IgG, 2 in 1-IgG, mAb2, or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (db), dsDb, DART, scDb, tandAbs, tandem scFv (taFv), tandem dAb/VHH, triple body, triple head, Fab-scFv, or F(ab′)2-scFv2), a bispecific Fc and CH3 fusion protein (e.g. a taFv-Fc, Di-diabody, scDb-CH3, scFv-Fc-scFv, HCAb-VHH, scFv-kih-Fc, or scFv-kih-CH3), or a bispecific fusion protein (e.g. a scFv2-albumin, scDb-albumin, taFv-toxin, DNL-Fab3, DNL-Fab4-IgG, DNL-Fab4-IgG-cytokine2). See in particular FIG. 2 of Kontermann MAbs 2012, 4(2): 182-19.

Methods for producing bispecific antibodies include chemically crosslinking antibodies or antibody fragments, e.g. with reducible disulphide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH-groups, to create disulfide-linked bispecific F(ab)2 heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen-binding fragments can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Färber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding to IL-11, an IL-11 containing complex, or a receptor for IL-11, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

Antibodies contemplated to be used in accordance with the present disclosure include antibodies having long-term persistence following administration to a subject. Antibodies may have pharmacokinetic properties such the antibody may be administered infrequently, e.g. once every ˜6 months. For example, antibodies according to the present disclosure may be engineered with Sequential Monoclonal Antibody Recycling Technology (SMART-Ig), as described e.g. in Fukuzawa et al., Sci Rep. (2017) 7(1):1080, which is hereby incorporated by reference in its entirety. Antibodies may be SMART recycling antibodies or SMART sweeping antibodies, engineered e.g. as described in Igawa et al., Nature Biotechnology (2010) 28:1203-1207 or Sampei et al., PLoS One (2018) 13(12):e0209509 (both hereby incorporated by reference in their entirety).

### Decoy Receptors

Peptide or polypeptide based agents capable of binding to IL-11 or IL-11 containing complexes may be based on the IL-11 receptor, e.g. an IL-11 binding fragment of an IL-11 receptor.

In some embodiments, the binding agent may comprise an IL-11-binding fragment of the IL-11Rα chain, and may preferably be soluble and/or exclude one or more, or all, of the transmembrane domain(s). In some embodiments, the binding agent may comprise an IL-11-binding fragment of gp130, and may preferably be soluble and/or exclude one or more, or all, of the transmembrane domain(s). Such molecules may be described as decoy receptors. Binding of such agents may inhibit IL-11 mediated cis and/or trans-signalling by reducing/preventing the ability of IL-11 to bind to receptors for IL-11, e.g. IL-11Rα or gp130, thereby inhibiting downstream signalling.

Curtis et al (Blood 1997 Dec. 1; 90 (11):4403-12) report that a soluble murine IL-11 receptor alpha chain (sIL-11R) was capable of antagonizing the activity of IL-11 when tested on cells expressing the transmembrane IL-11R and gp130. They proposed that the observed IL-11 antagonism by the sIL-11R depends on limiting numbers of gp130 molecules on cells already expressing the transmembrane IL-11R.

The use of soluble decoy receptors as the basis for inhibition of signal transduction and therapeutic intervention has also been reported for other signalling molecule:receptor pairs, e.g. VEGF and the VEGF receptor (De-Chao Yu et al., Molecular Therapy (2012); 20 5, 938-947; Konner and Dupont Clin Colorectal Cancer 2004 October; 4 Suppl 2:S81-5).

As such, in some embodiments a binding agent may be a decoy receptor, e.g. a soluble receptor for IL-11 and/or IL-11 containing complexes. Competition for IL-11 and/or IL-11 containing complexes provided by a decoy receptor has been reported to lead to IL-11 antagonist action (Curtis et al., supra). Decoy IL-11 receptors are also described in WO 2017/103108 A1 and WO 2018/109168 A1, which are hereby incorporated by reference in their entirety.

Decoy IL-11 receptors preferably bind IL-11 and/or IL-11 containing complexes, and thereby make these species unavailable for binding to gp130, IL-11Rα and/or gp130:IL-11Rα receptors. As such, they act as ‘decoy’ receptors for IL-11 and IL-11 containing complexes, much in the same way that etanercept acts as a decoy receptor for TNFα. IL-11-mediated signalling is reduced as compared to the level of signalling in the absence of the decoy receptor.

Decoy IL-11 receptors preferably bind to IL-11 through one or more cytokine binding modules (CBMs). The CBMs are, or are derived from or homologous to, the CBMs of naturally occurring receptor molecules for IL-11. For example, decoy IL-11 receptors may comprise, or consist of, one or more CBMs which are from, are derived from or homologous to the CBM of gp130 and/or IL-11Rα.

In some embodiments, a decoy IL-11 receptor may comprise, or consist of, an amino acid sequence corresponding to the cytokine binding module of gp130. In some embodiments, a decoy IL-11 receptor may comprise an amino acid sequence corresponding to the cytokine binding module of IL-11Rα. Herein, an amino acid sequence which ‘corresponds’ to a reference region or sequence of a given peptide/polypeptide has at least 60%, e.g. one of at least 65%, 70%, 75%, 80%, 85%, 90%, 91%, 92%, 93%, 94%, 95%, 96%, 97%, 98% or 99% sequence identity to the amino acid sequence of the reference region/sequence.

In some embodiments a decoy receptor may be able to bind IL-11, e.g. with binding affinity of at least 100 μM or less, optionally one of 10 μM or less, 1 μM or less, 100 nM or less, or about 1 to 100 nM. In some embodiments a decoy receptor may comprise all or part of the IL-11 binding domain and may optionally lack all or part of the transmembrane domains. The decoy receptor may optionally be fused to an immunoglobulin constant region, e.g. IgG Fc region.

### Inhibitors

The present disclosure contemplates the use of inhibitor molecules capable of binding to IL-11 or an IL-11 containing complex and inhibiting IL-11 mediated signalling. The present disclosure also contemplates the use of inhibitor molecules capable of binding to IL-11Rα, gp130, or a complex containing IL-11Rα and/or gp130, and inhibiting IL-11 mediated signalling.

In some embodiments the agent is a competitive inhibitor of IL-11. That is, in some embodiments the agent is an agent which competes with IL-11 and inhibits the action of IL-11. Competitive inhibitors of IL-11 include agents which compete with IL-11 for binding to IL-11 receptors (i.e. receptors comprising IL-11Rα, gp130, or a complex containing IL-11Rα and/or gp130).

Competitive inhibitors of IL-11 may be peptide- or polypeptide-based binding agents based on IL-11, e.g. mutant, variant or binding fragment of IL-11. Such agents may comprise an amino acid sequence having high sequence identity (e.g. 70%, 75%, 80%, 85% or greater) to the amino acid sequence of IL-11 or a fragment thereof. Suitable peptide or polypeptide based agents may bind to a receptor for IL-11 (e.g. IL-11Rα, gp130, or a complex containing IL-11Rα and/or gp130) in a manner that does not lead to initiation of signal transduction, or which produces sub-optimal signalling (i.e. a level of signalling which is reduced as compared to the level of signalling initiated by binding of wildtype IL-11). IL-11 variants and fragments of this kind may act as competitive inhibitors of endogenous IL-11.

For example, W147A is an IL-11 antagonist in which the amino acid 147 is mutated from a tryptophan to an alanine, which destroys the so-called ‘site III’ of IL-11. This mutant can bind to IL-11Rα, but engagement of the gp130 homodimer fails, resulting in efficient blockade of IL-11 signalling (Underhill-Day et al., 2003; Endocrinology 2003 August; 144(8):3406-14). Lee et al (Am J respire Cell Mol Biol. 2008 December; 39(6):739-746) also report the generation of an IL-11 antagonist mutant (a “mutein”) capable of specifically inhibiting the binding of IL-11 to IL-11Rα. IL-11 muteins are also described in WO 2009/052588 A1.

Menkhorst et al (Biology of Reproduction May 1, 2009 vol. 80 no. 5 920-927) describe a PEGylated IL-11 antagonist, PEGIL11A (CSL Limited, Parkvill, Victoria, Australia) which is effective to inhibit IL-11 action in female mice.

Pasqualini et al. Cancer (2015) 121(14):2411-2421 describe a ligand-directed, peptidomimetic drug, bone metastasis-targeting peptidomimetic-11 (BMTP-11) capable of binding to IL-11Rα.

In some embodiments a binding agent capable of binding to a receptor for IL-11 may be provided in the form of a small molecule inhibitor of one of IL-11Rα, gp130, or a complex containing IL-11Rα and/or gp130. In some embodiments a binding agent may be provided in the form of a small molecule inhibitor of IL-11 or an IL-11 containing complex, e.g. IL-11 inhibitor described in Lay et al., Int. J. Oncol. (2012); 41(2): 759-764, which is hereby incorporated by reference in its entirety.

### Aptamers

In some embodiments, an agent capable of binding to IL-11/an IL-11 containing complex or a receptor for IL-11 (e.g. IL-11Rα, gp130, or a complex containing IL-11Rα and/or gp130) is an aptamer. Aptamers, also called nucleic acid/peptide ligands, are nucleic acid or peptide molecules characterised by the ability to bind to a target molecule with high specificity and high affinity. Almost every aptamer identified to date is a non-naturally occurring molecule.

Aptamers to a given target (e.g. IL-11, an IL-11 containing complex or a receptor for IL-11) may be identified and/or produced by the method of Systematic Evolution of Ligands by EXponential enrichment (SELEX™), or by developing SOMAmers (slow off-rate modified aptamers) (Gold L et al. (2010) PLoS ONE 5(12):e15004). Aptamers and SELEX are described in Tuerk and Gold, Science (1990) 249(4968):505-10, and in WO 91/19813. Applying the SELEX and the SOMAmer technology includes for instance adding functional groups that mimic amino acid side chains to expand the aptamer's chemical diversity. As a result high affinity aptamers for a target may be enriched and identified.

Aptamers may be DNA or RNA molecules and may be single stranded or double-stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesised by methods which are well-known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12): 2223).

Suitable nucleic acid aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Suitable nucleic acid aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Suitable nucleic acid aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Aptamers may be peptides selected or engineered to bind specific target molecules. Peptide aptamers and methods for their generation and identification are reviewed in Reverdatto et al., Curr Top Med Chem. (2015) 15(12):1082-101, which is hereby incorporated by reference in its entirety. Peptide aptamers may optionally have a minimum length of one of 2, 3, 4, 5, 6, 7, 8, 9 or 10 amino acids. Peptide aptamers may optionally have a maximum length of one of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. Suitable peptide aptamers may optionally have a length of one of 2-30, 2-25, 2-20, 5-30, 5-25 or 5-20 amino acids.

Aptamers may have KD's in the nM or pM range, e.g. less than one of 500 nM, 100 nM, 50 nM, 10 nM, 1 nM, 500 pM, 100 pM.

### Properties of IL-11 Binding Agents

Agents capable of binding to IL-11/an IL-11 containing complex or a receptor for IL-11 according to the present disclosure may exhibit one or more of the following properties:
Specific binding to IL-11/IL-11 containing complex or a receptor for IL-11;
Binding to IL-11/IL-11 containing complex, or a receptor for IL-11, with a KD of 10 μM or less, preferably one of ≤5 μM≤1 μM, ≤500 nM, ≤100 nM, ≤10 nM, ≤1 nM or ≤100 pM;
Inhibition of interaction between IL-11 and IL-11Rα;
Inhibition of interaction between IL-11 and gp130;
Inhibition of interaction between IL-11 and IL-11Rα:gp130 receptor complex;
Inhibition of interaction between IL-11:IL-11Rα complex and gp130; and
Inhibition of interaction between IL-11:IL-11Rα:gp130 complexes (i.e. multimerisation of such complexes).

These properties can be determined by analysis of the relevant agent in a suitable assay, which may involve comparison of the performance of the agent to suitable control agents. The skilled person is able to identify an appropriate control conditions for a given assay.

For example, a suitable negative control for the analysis of the ability of a test antibody/antigen-binding fragment to bind to IL-11/an IL-11 containing complex/a receptor for IL-11 may be an antibody/antigen-binding fragment directed against a non-target protein (i.e. an antibody/antigen-binding fragment which is not specific for IL-11/an IL-11 containing complex/a receptor for IL-11). A suitable positive control may be a known, validated (e.g. commercially available) IL-11- or IL-11 receptor-binding antibody. Controls may be of the same isotype as the putative IL-11/IL-11 containing complex/IL-11 receptor-binding antibody/antigen-binding fragment being analysed, and may e.g. have the same constant regions.

In some embodiments, the agent may be capable of binding specifically to IL-11 or an IL-11 containing complex, or a receptor for IL-11 (e.g. IL-11Rα, gp130, or a complex containing IL-11Rα and/or gp130). An agent which specifically binds to a given target molecule preferably binds the target with greater affinity, and/or with greater duration than it binds to other, non-target molecules.

In some embodiments the agent may bind to IL-11 or an IL-11 containing complex with greater affinity than the affinity of binding to one or more other members of the IL-6 cytokine family (e.g. IL-6, leukemia inhibitory factor (LIF), oncostatin M (OSM), cardiotrophin-1 (CT-1), ciliary neurotrophic factor (CNTF) and cardiotrophin-like cytokine (CLC)). In some embodiments the agent may bind to a receptor for IL-11 (e.g. IL-11Rα, gp130, or a complex containing IL-11Rα and/or gp130) with greater affinity than the affinity of binding to one or more other members of the IL-6 receptor family. In some embodiments the agent may bind with greater affinity to IL-11Rα than the affinity of binding to one or more of IL-6Rα, leukemia inhibitory factor receptor (LIFR), oncostatin M receptor (OSMR), ciliary neurotrophic factor receptor alpha (CNTFRα) and cytokine receptor-like factor 1 (CRLF1).

In some embodiments, the extent of binding of a binding agent to an non-target is less than about 10% of the binding of the agent to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the binding agent binds to IL-11, an IL-11 containing complex or a receptor for IL-11 with a K_{D} that is at least 0.1 order of magnitude (i.e. 0.1×10n, where n is an integer representing the order of magnitude) greater than the K_{D} towards another, non-target molecule. This may optionally be one of at least 0.2, 0.3, 0.4, 0.5, 0.6, 0.7, 0.8, 0.9, 1.0, 1.5, or 2.0.

Binding affinity for a given binding agent for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4): 342-353), or by a radiolabelled antigen binding assay (RIA).

In some embodiments, the agent is capable of binding to IL-11 or an IL-11 containing complex, or a receptor for IL-11 with a K_{D} of 50 μM or less, preferably one of ≤10 μM, ≤5 μM, ≤4 μM, ≤3 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 μM, ≤400 μM, ≤300 μM, ≤200 μM, or ≤100 μM.

In some embodiments, the agent binds to IL-11, an IL-11 containing complex or a receptor for IL-11 with an affinity of binding (e.g. as determined by ELISA) of EC50=10,000 ng/ml or less, preferably one of ≤5,000 ng/ml, ≤1000 ng/ml, ≤900 ng/ml, ≤800 ng/ml, ≤700 ng/ml, ≤600 ng/ml, ≤500 ng/ml, ≤400 ng/ml, 5300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, 530 ng/ml, ≤20 ng/ml, ≤15 ng/ml, ≤10 ng/ml, ≤7.5 ng/ml, ≤5 ng/ml, ≤2.5 ng/ml, or ≤1 ng/ml. Such ELISAs can be performed e.g. as described in Antibody Engineering, vol. 1 (2nd Edn), Springer Protocols, Springer (2010), Part V, pp 657-665.

In some embodiments, the agent binds to IL-11 or an IL-11-containing complex in a region which is important for binding to a receptor for the IL-11 or IL-11-containing complex, e.g. gp130 or IL-11Rα, and thereby inhibits interaction between IL-11 or an IL-11-containing complex and a receptor for IL-11, and/or signalling through the receptor. In some embodiments, the agent binds to a receptor for IL-11 in a region which is important for binding to IL-11 or an IL-11-containing complex, and thereby inhibits interaction between IL-11 or an IL-11-containing complex and a receptor for IL-11, and/or signalling through the receptor.

The ability of a given binding agent (e.g. an agent capable of binding IL-11/an IL-11 containing complex or a receptor for IL-11) to inhibit interaction between two proteins can be determined for example by analysis of interaction in the presence of, or following incubation of one or both of the interaction partners with, the binding agent. An example of a suitable assay to determine whether a given binding agent is capable of inhibiting interaction between two interaction partners is a competition ELISA.

A binding agent which is capable of inhibiting a given interaction (e.g. between IL-11 and IL-11Rα, or between IL-11 and gp130, or between IL-11 and IL-11Rα:gp130, or between IL-11:IL-11Rα and gp130, or between IL-11:IL-11Rα:gp130 complexes) is identified by the observation of a reduction/decrease in the level of interaction between the interaction partners in the presence of—or following incubation of one or both of the interaction partners with—the binding agent, as compared to the level of interaction in the absence of the binding agent (or in the presence of an appropriate control binding agent). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the binding agent may be labelled or used in conjunction with a detectable entity for the purposes of detecting and/or measuring the level of interaction. For example, the agent may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding agent may be directly labelled with a detectable label or it may be indirectly labelled. For example, the binding agent may be unlabelled, and detected by another binding agent which is itself labelled. Alternatively, the second binding agent may have bound to it biotin and binding of labelled streptavidin to the biotin may be used to indirectly label the first binding agent.

Ability of a binding agent to inhibit interaction between two binding partners can also be determined by analysis of the downstream functional consequences of such interaction, e.g. IL-11-mediated signalling. For example, downstream functional consequences of interaction between IL-11 and IL-11Rα:gp130 or between IL-11:IL-11Rα and gp130, or between IL-11:IL-11Rα:gp130 complexes may include e.g. a process mediated by IL-11, or gene/protein expression of e.g. IL-11.

Inhibition of interaction between IL-11 or an IL-11 containing complex and a receptor for IL-11 can be analysed using 3H-thymidine incorporation and/or Ba/F3 cell proliferation assays such as those described in e.g. Curtis et al. Blood, 1997, 90(11) and Karpovich et al. Mol. Hum. Reprod. 2003 9(2): 75-80. Ba/F3 cells co-express IL-11Rα and gp130.

In some embodiments, the binding agent may be capable of inhibiting interaction between IL-11 and IL-11Rα to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα in the absence of the binding agent (or in the presence of an appropriate control binding agent). In some embodiments, the binding agent may be capable of inhibiting interaction between IL-11 and IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα in the absence of the binding agent (or in the presence of an appropriate control binding agent).

In some embodiments, the binding agent may be capable of inhibiting interaction between IL-11 and gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and gp130 in the absence of the binding agent (or in the presence of an appropriate control binding agent). In some embodiments, the binding agent may be capable of inhibiting interaction between IL-11 and gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and gp130 in the absence of the binding agent (or in the presence of an appropriate control binding agent).

In some embodiments, the binding agent may be capable of inhibiting interaction between IL-11 and IL-11Rα:gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα:gp130 in the absence of the binding agent (or in the presence of an appropriate control binding agent). In some embodiments, the binding agent may be capable of inhibiting interaction between IL-11 and IL-11Rα:gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα:gp130 in the absence of the binding agent (or in the presence of an appropriate control binding agent).

In some embodiments, the binding agent may be capable of inhibiting interaction between IL-11:IL-11Rα complex and gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11:IL-11Rα complex and gp130 in the absence of the binding agent (or in the presence of an appropriate control binding agent). In some embodiments, the binding agent is capable of inhibiting interaction between IL-11:IL-11Rα complex and gp130 to less than 1 times, e.g. one of ≤0.99 times, ≤0.95 times, 50.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα complex and gp130 in the absence of the binding agent.

In some embodiments, the binding agent may be capable of inhibiting interaction between IL-11:IL-11Rα:gp130 complexes (i.e. multimerisation of such complexes) to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11:IL-11Rα:gp130 complexes in the absence of the binding agent (or in the presence of an appropriate control binding agent). In some embodiments, the binding agent is capable of inhibiting interaction between IL-11:IL-11Rα:gp130 complexes to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα:gp130 complexes in the absence of the binding agent.

### Agents Capable of Reducing Expression of IL-11 or an IL-11 Receptor

In aspects of the present disclosure the agent capable of inhibiting IL-11-mediated signalling may be capable of preventing or reducing the expression of one or more of IL-11, IL-11Rα or gp130.

Expression may be gene or protein expression, and may be determined as described herein or by methods in the art that will be well-known to a skilled person. Expression may be by a cell/tissue/organ/organ system of a subject.

Suitable agents may be of any kind, but in some embodiments an agent capable of preventing or reducing the expression of one or more of IL-11, IL-11Rα or gp130 may be a small molecule or an oligonucleotide.

An agent capable of preventing or reducing of the expression of one or more of IL-11, IL-11Rα or gp130 may do so e.g. through inhibiting transcription of the gene encoding IL-11, IL-11Rα or gp130, inhibiting post-transcriptional processing of RNA encoding IL-11, IL-11Rα or gp130, reducing the stability of RNA encoding IL-11, IL-11Rα or gp130, promoting degradation of RNA encoding IL-11, IL-11Rα or gp130, inhibiting post-translational processing of IL-11, IL-11Rα or gp130 polypeptide, reducing the stability of IL-11, IL-11Rα or gp130 polypeptide or promoting degradation of IL-11, IL-11Rα or gp130 polypeptide.

Taki et al. Clin Exp Immunol (1998) April; 112(1): 133-138 reported a reduction in the expression of IL-11 in rheumatoid synovial cells upon treatment with indomethacin, dexamethasone or interferon-gamma (IFNγ).

The present disclosure contemplates the use of antisense nucleic acid to prevent/reduce expression of IL-11, IL-11Rα or gp130. In some embodiments, an agent capable of preventing or reducing the expression of IL-11, IL-11Rα or gp130 may cause reduced expression by RNA interference (RNAi).

In some embodiments, the agent may be an inhibitory nucleic acid, such as antisense or small interfering RNA, including but not limited to shRNA or siRNA.

In some embodiments the inhibitory nucleic acid is provided in a vector. For example, in some embodiments the agent may be a lentiviral vector encoding shRNA for one or more of IL-11, IL-11Rα or gp130.

Oligonucleotide molecules, particularly RNA, may be employed to regulate gene expression. These include antisense oligonucleotides, targeted degradation of mRNAs by small interfering RNAs (siRNAs), post-transcriptional gene silencing (PTGs), developmentally regulated sequence-specific translational repression of mRNA by micro-RNAs (miRNAs) and targeted transcriptional gene silencing.

An antisense oligonucleotide is an oligonucleotide, preferably single-stranded, that targets and binds, by complementary sequence binding, to a target oligonucleotide, e.g. mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription of a target nucleotide sequence.

In view of the known nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130)) oligonucleotides may be designed to repress or silence the expression of IL-11, IL-11Rα or gp130.

Such oligonucleotides may have any length, but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 98%, 99% or 100% complementarity) to a sequence of nucleotides of corresponding length in the target oligonucleotide, e.g. the IL-11, IL-11Rα or gp130 mRNA. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Repression of expression of IL-11, IL-11Rα or gp130 will preferably result in a decrease in the quantity of IL-11, IL-11Rα or gp130 expressed by a cell/tissue/organ/organ system/subject. For example, in a given cell the repression of IL-11, IL-11Rα or gp130 by administration of a suitable nucleic acid will result in a decrease in the quantity of IL-11, IL-11Rα or gp130 expressed by that cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post-transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457(7228):426-433).

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double-stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

siRNA ligands are typically double-stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single-stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double-stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double-stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of IL-11, IL-11Rα or gp130. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of IL-11, IL-11Rα or gp130.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g. heart, liver, or kidney specific) promoter. In a further embodiment, the siRNA, longer dsRNA or miRNA is produced exogenously (in vitro) by transcription from a vector.

Suitable vectors may be oligonucleotide vectors configured to express the oligonucleotide agent capable of IL-11, IL-11Rα or gp130 repression. Such vectors may be viral vectors or plasmid vectors. The therapeutic oligonucleotide may be incorporated in the genome of a viral vector and be operably linked to a regulatory sequence, e.g. promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long term expression of the therapeutic oligonucleotide. Examples include lentiviral (Nature 2009 Jan. 22; 457(7228):426-433), adenovirus (Shen et al., FEBS Lett 2003 Mar. 27; 539(1-3)111-4) and retroviruses (Barton and Medzhitov PNAS Nov. 12, 2002 vol. 99, no. 23 14943-14945).

In other embodiments a vector may be configured to assist delivery of the therapeutic oligonucleotide to the site at which repression of IL-11, IL-11Rα or gp130 expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, and lipids), polymers, antibodies, and RNAs; or encapsulating the oligonucleotide in nanoparticulate formulations (Wang et al., AAPS J. 2010 December; 12(4): 492-503).

In one embodiment, a vector may comprise a nucleic acid sequence in both the sense and antisense orientation, such that when expressed as RNA the sense and antisense sections will associate to form a double-stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through-O-or-S-.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, pseudouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6, diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Accordingly, the present disclosure provides nucleic acid that is capable, when suitably introduced into or expressed within a mammalian, e.g. human, cell that otherwise expresses IL-11, IL-11Rα or gp130, of suppressing IL-11, IL-11Rα or gp130 expression by RNAi.

Nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130)) oligonucleotides may be designed to repress or silence the expression of IL-11, IL-11Rα or gp130.

The nucleic acid may have substantial sequence identity to a portion of IL-11, IL-11Rα or gp130 mRNA, e.g. as defined in GenBank accession no. NM_000641.3 GI:391353405 (IL-11), NM_001142784.2 GI:391353394 (IL-11Rα), NM_001190981.1 GI:300244534 (gp130) or the complementary sequence to said mRNA.

The nucleic acid may be a double-stranded siRNA. (As the skilled person will appreciate, and as explained further below, a siRNA molecule may include a short 3′ DNA sequence also.)

Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridise with each other. In a mammalian cell, the hairpin structure may be cleaved from the molecule by the enzyme DICER, to yield two distinct, but hybridised, RNA molecules.

In some preferred embodiments, the nucleic acid is generally targeted to the sequence of one of SEQ ID NOs 4 to 7 (IL-11) or to one of SEQ ID NOs 8 to 11 (IL-11Rα).

Only single-stranded (i.e. non self-hybridised) regions of an mRNA transcript are expected to be suitable targets for RNAi. It is therefore proposed that other sequences very close in the IL-11 or IL-11Rα mRNA transcript to the sequence represented by one of SEQ ID NOs 4 to 7 or 8 to 11 may also be suitable targets for RNAi. Such target sequences are preferably 17-23 nucleotides in length and preferably overlap one of SEQ ID NOs 4 to 7 or 8 to 11 by at least 1, 2, 3, 4, 5, 6, 7, 8, 9, 10, 11, 12, 13, 14, 15, 16, 17, 18 or all 19 nucleotides (at either end of one of SEQ ID NOs 4 to 7 or 8 to 11).

Accordingly, the present disclosure provides nucleic acid that is capable, when suitably introduced into or expressed within a mammalian cell that otherwise expresses IL-11 or IL-11Rα, of suppressing IL-11 or IL-11Rα expression by RNAi, wherein the nucleic acid is generally targeted to the sequence of one of SEQ ID NOs 4 to 7 or 8 to 11.

By “generally targeted” the nucleic acid may target a sequence that overlaps with SEQ ID NOs 4 to 7 or 8 to 11. In particular, the nucleic acid may target a sequence in the mRNA of human IL-11 or IL-11Rα that is slightly longer or shorter than one of SEQ ID NOs 4 to 7 or 8 to 11 (preferably from 17-23 nucleotides in length), but is otherwise identical to one of SEQ ID NOs 4 to 7 or 8 to 11.

It is expected that perfect identity/complementarity between the nucleic acid of the present disclosure and the target sequence, although preferred, is not essential. Accordingly, the nucleic acid of the present disclosure may include a single mismatch compared to the mRNA of IL-11 or IL-11Rα. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the present disclosure that include non-natural nucleotides.

In one embodiment, the nucleic acid (herein referred to as double-stranded siRNA) includes the double-stranded RNA sequences shown in SEQ ID NOs 12 to 15. In another embodiment, the nucleic acid (herein referred to as double-stranded siRNA) includes the double-stranded RNA sequences shown in SEQ ID NOs 16 to 19.

However, it is also expected that slightly shorter or longer sequences directed to the same region of IL-11 or IL-11Rα mRNA will also be effective. In particular, it is expected that double-stranded sequences between 17 and 23 bp in length will also be effective.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c).

While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred.

Moreover, the 3′ overhangs may be omitted entirely from the siRNA.

The present disclosure also provides single-stranded nucleic acids (herein referred to as single-stranded siRNAs) respectively consisting of a component strand of one of the aforementioned double-stranded nucleic acids, preferably with the 3′-overhangs, but optionally without. The present disclosure also provides kits containing pairs of such single-stranded nucleic acids, which are capable of hybridising with each other in vitro to form the aforementioned double-stranded siRNAs, which may then be introduced into cells.

The present disclosure also provides DNA that, when transcribed in a mammalian cell, yields an RNA (herein also referred to as an shRNA) having two complementary portions which are capable of self-hybridising to produce a double-stranded motif, e.g. including a sequence selected from the group consisting of SEQ ID NOs: 12 to 15 or 16 to 19 or a sequence that differs from any one of the aforementioned sequences by a single base pair substitution.

The complementary portions will generally be joined by a spacer, which has suitable length and sequence to allow the two complementary portions to hybridise with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Washington, USA) is UUCAAGAGA. In this and other cases, the ends of the spacer may hybridise with each other, e.g. elongating the double-stranded motif beyond the exact sequences of SEQ ID NOs 12 to 15 or 16 to 19 by a small number (e.g. 1 or 2) of base pairs.

Similarly, the transcribed RNA preferably includes a 3′ overhang from the downstream complementary portion. Again, this is preferably -UU or -UG, more preferably -UU.

Such shRNA molecules may then be cleaved in the mammalian cell by the enzyme DICER to yield a double-stranded siRNA as described above, in which one or each strand of the hybridised dsRNA includes a 3′ overhang.

Techniques for the synthesis of the nucleic acids of the present disclosure are of course well-known in the art.

The skilled person is well able to construct suitable transcription vectors for the DNA of the present disclosure using well-known techniques and commercially available materials. In particular, the DNA will be associated with control sequences, including a promoter and a transcription termination sequence.

Of particular suitability are the commercially available pSuper and pSuperior systems of OligoEngine (Seattle, Washington, USA). These use a polymerase-III promoter (H1) and a T5 transcription terminator sequence that contributes two U residues at the 3′ end of the transcript (which, after DICER processing, provide a 3′ UU overhang of one strand of the siRNA).

Another suitable system is described in Shin et al. (RNA, 2009 May; 15(5): 898-910), which uses another polymerase-III promoter (U6).

The double-stranded siRNAs of the present disclosure may be introduced into mammalian cells in vitro or in vivo using known techniques, as described below, to suppress expression of IL-11 or a receptor for IL-11.

Similarly, transcription vectors containing the DNAs of the present disclosure may be introduced into tumour cells in vitro or in vivo using known techniques, as described below, for transient or stable expression of RNA, again to suppress expression of IL-11 or a receptor for IL-11.

Accordingly, the present disclosure also provides a method of suppressing expression of IL-11 or a receptor for IL-11 in a mammalian, e.g. human, cell, the method comprising administering to the cell a double-stranded siRNA of the present disclosure or a transcription vector of the present disclosure.

Similarly, the present disclosure further provides a method of treating diseases/conditions described herein, comprising administering to a subject a double-stranded siRNA of the present disclosure or a transcription vector of the present disclosure.

The present disclosure further provides the double-stranded siRNAs of the present disclosure and the transcription vectors of the present disclosure, for use in a method of treating/preventing a disease/condition in accordance with the present disclosure.

The present disclosure further provides the use of the double-stranded siRNAs of the present disclosure and the transcription vectors of the present disclosure in the preparation of a medicament for the treatment/prevention of a disease/condition described herein.

The present disclosure further provides a composition comprising a double-stranded siRNA of the present disclosure or a transcription vector of the present disclosure in admixture with one or more pharmaceutically acceptable carriers. Suitable carriers include lipophilic carriers or vesicles, which may assist in penetration of the cell membrane.

Materials and methods suitable for the administration of siRNA duplexes and DNA vectors of the present disclosure are well-known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE, dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210).

In particular, suitable techniques for cellular administration of the nucleic acids of the present disclosure both in vitro and in vivo are disclosed in the following articles:

General reviews: Borkhardt, A. 2002. Blocking oncogenes in malignant cells by RNA interference—new hope for a highly specific cancer treatment? Cancer Cell. 2:167-8. Hannon, G. J. 2002. RNA interference. Nature. 418:244-51. McManus, M. T., and P. A. Sharp. 2002. Gene silencing in mammals by small interfering RNAs. Nat Rev Genet. 3:737-47. Scherr, M., M. A. Morgan, and M. Eder. 2003b. Gene silencing mediated by small interfering RNAs in mammalian cells. Curr Med Chem. 10:245-56. Shuey, D. J., D. E. McCallus, and T. Giordano. 2002. RNAi: gene-silencing in therapeutic intervention. Drug Discov Today. 7:1040-6.

Systemic delivery using liposomes: Lewis, D. L., J. E. Hagstrom, A. G. Loomis, J. A. Wolff, and H. Herweijer. 2002. Efficient delivery of siRNA for inhibition of gene expression in postnatal mice. Nat Genet. 32:107-8. Paul, C. P., P. D. Good, I. Winer, and D. R. Engelke. 2002. Effective expression of small interfering RNA in human cells. Nat Biotechnol. 20:505-8. Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51. Sorensen, D. R., M. Leirdal, and M. Sioud. 2003. Gene silencing by systemic delivery of synthetic siRNAs in adult mice. J Mol Biol. 327:761-6.

Virus mediated transfer: Abbas-Terki, T., W. Blanco-Bose, N. Deglon, W. Pralong, and P. Aebischer. 2002. Lentiviral-mediated RNA interference. Hum Gene Ther. 13:2197-201. Barton, G. M., and R. Medzhitov. 2002. Retroviral delivery of small interfering RNA into primary cells. Proc Natl Acad Sci USA. 99:14943-5. Devroe, E., and P. A. Silver. 2002. Retrovirus-delivered siRNA. BMC Biotechnol. 2:15. Lori, F., P. Guallini, L. Galluzzi, and J. Lisziewicz. 2002. Gene therapy approaches to HIV infection. Am J Pharmacogenomics. 2:245-52. Matta, H., B. Hozayev, R. Tomar, P. Chugh, and P. M. Chaudhary. 2003. Use of lentiviral vectors for delivery of small interfering RNA. Cancer Biol Ther. 2:206-10. Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci USA. 100:183-8. Scherr, M., K. Battmer, A. Ganser, and M. Eder. 2003a. Modulation of gene expression by lentiviral-mediated delivery of small interfering RNA. Cell Cycle. 2:251-7. Shen, C., A. K. Buck, X. Liu, M. Winkler, and S. N. Reske. 2003. Gene silencing by adenovirus-delivered siRNA. FEBS Lett. 539:111-4.

Peptide delivery: Morris, M. C., L. Chaloin, F. Heitz, and G. Divita. 2000. Translocating peptides and proteins and their use for gene delivery. Curr Opin Biotechnol. 11:461-6. Simeoni, F., M. C. Morris, F. Heitz, and G. Divita. 2003. Insight into the mechanism of the peptide-based gene delivery system MPG: implications for delivery of siRNA into mammalian cells. Nucleic Acids Res. 31:2717-24. Other technologies that may be suitable for delivery of siRNA to the target cells are based on nanoparticles or nanocapsules such as those described in US patent numbers 6,649,192B and 5,843,509B.

### Inhibition of IL-11-Mediated Signalling

In embodiments of the present disclosure, agents capable of inhibiting the action of IL-11 may possess one or more of the following functional properties:
Inhibition of signalling mediated by IL-11;
Inhibition of signalling mediated by binding of IL-11 to IL-11Rα:gp130 receptor complex;
Inhibition of signalling mediated by binding of IL-11:IL-11Rα complex to gp130 (i.e. IL-11 trans signalling);
Inhibition of signalling mediated by multimerisation of IL-11:IL-11Rα:gp130 complexes;
Inhibition of a process mediated by IL-11;
Inhibition of gene/protein expression of IL-11 and/or IL-11Rα.

These properties can be determined by analysis of the relevant agent in a suitable assay, which may involve comparison of the performance of the agent to suitable control agents. The skilled person is able to identify an appropriate control conditions for a given assay.

IL-11-mediated signalling and/or processes mediated by IL-11 includes signalling mediated by fragments of IL-11 and polypeptide complexes comprising IL-11 or fragments thereof. IL-11-mediated signalling may be signalling mediated by human IL-11 and/or mouse IL-11. Signalling mediated by IL-11 may occur following binding of IL-11 or an IL-11 containing complex to a receptor to which IL-11 or said complex binds.

In some embodiments, an agent may be capable of inhibiting the biological activity of IL-11 or an IL-11-containing complex.

In some embodiments, the agent is an antagonist of one or more signalling pathways which are activated by signal transduction through receptors comprising IL-11Rα and/or gp130, e.g. IL-11Rα:gp130. In some embodiments, the agent is capable of inhibiting signalling through one or more immune receptor complexes comprising IL-11Rα and/or gp130, e.g. IL-11Rα:gp130. In various aspects of the present disclosure, an agent provided herein is capable of inhibiting IL-11-mediated cis and/or trans signalling. In some embodiments in accordance with the various aspects of the present disclosure, an agent provided herein is capable of inhibiting IL-11-mediated cis signalling.

In some embodiments, the agent may be capable of inhibiting IL-11-mediated signalling to less than 100%, e.g. one of 99% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of signalling in the absence of the agent (or in the presence of an appropriate control agent). In some embodiments, the agent is capable of reducing IL-11-mediated signalling to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of signalling in the absence of the agent (or in the presence of an appropriate control agent).

In some embodiments, the IL-11-mediated signalling may be signalling mediated by binding of IL-11 to IL-11Rα:gp130 receptor. Such signalling can be analysed e.g. by treating cells expressing IL-11Rα and gp130 with IL-11, or by stimulating IL-11 production in cells which express IL-11Rα and gp130.

The IC₅₀ for an agent for inhibition of IL-11-mediated signalling may be determined, e.g. by culturing Ba/F3 cells expressing IL-11Rα and gp130 in the presence of human IL-11 and the agent, and measuring 3H-thymidine incorporation into DNA. In some embodiments, the agent may exhibit an IC₅₀ of 10 μg/ml or less, preferably one of ≤5 μg/ml, ≤4 μg/ml, ≤3.5 μg/ml, ≤3 μg/ml, ≤2 μg/ml, ≤1 μg/ml, ≤0.9 μg/ml, ≤0.8 μg/ml, ≤0.7 μg/ml, ≤0.6 μg/ml, or ≤0.5 μg/ml in such an assay.

In some embodiments, the IL-11-mediated signalling may be signalling mediated by binding of IL-11:IL-11Rα complex to gp130. In some embodiments, the IL-11:IL-11Rα complex may be soluble, e.g. complex of extracellular domain of IL-11Rα and IL-11, or complex of soluble IL-11Rα isoform/fragment and IL-11. In some embodiments, the soluble IL-11Rα is a soluble (secreted) isoform of IL-11Rα, or is the liberated product of proteolytic cleavage of the extracellular domain of cell membrane bound IL-11Rα.

In some embodiments, the IL-11:IL-11Rα complex may be cell-bound, e.g. complex of cell-membrane bound IL-11Rα and IL-11. Signalling mediated by binding of IL-11:IL-11Rα complex to gp130 can be analysed by treating cells expressing gp130 with IL-11:IL-11Rα complex, e.g. recombinant fusion protein comprising IL-11 joined by a peptide linker to the extracellular domain of IL-11Rα, e.g. hyper IL-11. Hyper IL-11 was constructed using fragments of IL-11Rα (amino acid residues 1 to 317 consisting of domain 1 to 3; UniProtKB: Q14626) and IL-11 (amino acid residues 22 to 199 of UniProtKB: P20809) with a 20 amino acid long linker (SEQ ID NO:20). The amino acid sequence for Hyper IL-11 is shown in SEQ ID NO:21.

In some embodiments, the agent may be capable of inhibiting signalling mediated by binding of IL-11:IL-11Rα complex to gp130, and is also capable of inhibiting signalling mediated by binding of IL-11 to IL-11Rα:gp130 receptor.

In some embodiments, the agent may be capable of inhibiting a process mediated by IL-11.

In some embodiments, the agent may be capable of inhibiting gene/protein expression of IL-11 and/or IL-11Rα. Gene and/or protein expression can be measured as described herein or by methods in the art that will be well-known to a skilled person.

In some embodiments, the agent may be capable of inhibiting gene/protein expression of IL-11 and/or IL-11Rα to less than 100%, e.g. one of 99% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of expression in the absence of the agent (or in the presence of an appropriate control agent). In some embodiments, the agent is capable of inhibiting gene/protein expression of IL-11 and/or IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of expression in the absence of the agent (or in the presence of an appropriate control agent).

### Therapeutic and Prophylactic Indications

The present disclosure is broadly concerned with the treatment/prevention of age-related diseases/conditions. Age-related diseases/conditions as referred to herein are diseases/conditions having an incidence which increases with age. Age-related diseases and conditions are described e.g. in Franceschi et al., Front Med (Lausanne) (2018) 5: 61 and Jaul and Barron Front Public Health (2017) 5: 335, both of which are hereby incorporated by reference in their entirety.

Age-related diseases/conditions are typically characterised by progressive degeneration of tissue structure and/or the progressive decline of physiological tissue function. The molecular and cellular mechanisms underlying such diseases/conditions include one or more of deregulated autophagy, mitochondrial dysfunction, telomere shortening, oxidative stress, inflammation, metabolic dysfunction, and commonly cellular senescence.

Aging is a major risk factor for many chronic diseases. In the liver, aging increases the susceptibility towards acute liver injury and hepatic fibrotic response (Kim et al., Curr Opin Gastroenterol (2015) 31(3): 184-191; Hunt et al., Comput Struct Biotechnol J (2019) 17: 1151-1161, Ferrucci et al., Aging Cell (2020) 19(2): e13080). Moreover, aging has been positively associated with increased risk and poor prognosis of various liver diseases including non-alcoholic fatty liver disease (NAFLD), alcoholic liver disease, hepatitis C, and negatively associated with hepatic regenerative capacity (Kim et al., Curr Opin Gastroenterol (2015) 31(3): 184-191; Papatheodoridi et al., Hepatology (2020) 71(1): 363-374).

As used herein, an “age-related” disease/condition or phenotype may also be referred to as being “aging-related” or “age/aging-associated”. In aspects and embodiments of the present disclosure, a disease/condition or phenotype which is described as being “age-related” may arise as a consequence of the age of the subject having the relevant disease/condition or phenotype, rather than another etiological cause. In some aspects and embodiments, a disease/condition or phenotype which is “age-related” may arise as a consequence of cellular senescence. By way of illustration, “age-related” changes in body composition may refer to changes in body composition arising as a consequence of aging of the subject and/or of cellular senescence, rather than the subject's diet.

The accumulation of senescent cells is one of the hallmarks in aging (Hunt et al., Comput Struct Biotechnol J (2019) 17: 1151-1161). Cellular senescence is characterised by reduced replicative capacity and producing senescence-associated secretory phenotype (SASPs) proteins, resulting in a chronic, low-grade inflammatory environment for neighbouring cells (Hunt et al., Comput Struct Biotechnol J (2019) 17: 1151-1161; Borghesan et al., Trends Cell Biol. (2020) 30(10):777-791). Under pathological stress conditions, excessive accumulation of senescence cells in affected tissues adversely affects the tissue's regenerative ability and chronic inflammation that can resemble various age-related disorders such as Alzheimer's disease, cancer, arthritis, cataracts, osteoporosis, atherosclerosis, hypertension, cardiovascular disease, type 2 diabetes, and chronic liver disorders (Baker and Haynes, Trends Biochem Sci (2011) 36(5): 254-261; Kim et al., Curr Opin Gastroenterol (2015) 31(3): 184-191; Hernandez-Segura et al., Trends Cell Biol (2018) 28(6): 436-453; Stahl et al. Front Immunol (2018) 9: 2795; Belikov, Ageing Res Rev (2019) 49: 11-26; Campisi et al., Nature (2019) 571(7764): 183-192; Gorgoulis et al., Cell (2019) 179(4): 813-827; Schmeer et al., Cells (2019) 8(11); Papatheodoridi et al., Hepatology (2020) 71(1): 363-374). Cellular senescence is therefore thought to be a key physiological process in the development and progression of age-related diseases (Borghesan et al., Trends Cell Biol. (2020) 30(10):777-791; Pignolo et al., Trends Mol Med (2020) 26(7): 630-638).

In some aspects and embodiments the present disclosure contemplates treatment/prevention of cellular senescence, and diseases/conditions characterised by cellular senescence. In some aspects and embodiments, the methods of the present disclosure comprise inhibiting cellular senescence. In some aspects and embodiments, the methods of the present disclosure comprise inhibiting senescent cells. In some aspects and embodiments, the methods comprise reducing the number of senescent cells and/or inhibiting the activity of senescent cells.

In some embodiments, reducing the number of senescent cells comprises inhibiting the process of cellular senescence. That is, in some embodiments reducing the number of senescent cells comprises inhibiting the development of senescent cells from non-senescent precursor cells. In some embodiments, reducing the number of senescent cells comprises reversing the process of cellular senescence. That is, in some embodiments reducing the number of senescent cells comprises promoting reversion of senescent cells to a non-senescent phenotype. In some embodiments, reducing the number of senescent cells comprises depleting senescent cells.

Cellular senescence is described e.g. in Childs et al. Nat Med (2015) 21(12):1424-1435 and van Deursen, Nature (2014) 509(7501): 439-446, both of which are hereby incorporated by reference in their entirety. Cellular senescence is characterised by cessation of cell division (associated with activation of p16^{INK4a}p21^{CIP1} and p53), chromatin remodelling (including e.g. DNA damage response (DDR), formation of promyelocytic leukemia protein (PML) bodies and senescence associated heterochromatic foci (SAHF)), senescence-associated p-galactosidase activity, and production of a mixture of proinflammatory factors termed the senescence-associated secretory phenotype (SASP).

In accordance with the present disclosure, a senescent cell may display one or more of the following relative to an equivalent non-senescent cell of the same cell type/from the same tissue: increased expression of p16^{INK4a}, p21^{CIP1} and/or p53; increased level of DDR; increased number of PML bodies; increased number of SAHF, increased expression and/or activity of senescence-associated β-galactosidase; increased expression of one or more SASP factors (e.g. IL-1b or IL-8).

In particular aspects and embodiments, the present disclosure relates to the treatment of diseases/conditions comprising and/or characterised by cellular senescence, i.e. diseases/conditions in which cellular senescence is pathologically-implicated. A disease/condition in which cellular senescence is ‘pathologically-implicated’ is a disease/condition in which the number/proportion and/or activity of senescent cells is positively associated with the disease or condition.

A disease/condition in which cellular senescence is ‘pathologically-implicated’ may be a disease/condition for which an increase in the number/proportion and/or activity of senescent cells (relative to the non-diseased, healthy state) is positively associated with the onset, development and/or progression of the disease/condition. A disease/condition in which cellular senescence is ‘pathologically-implicated’ may be a disease/condition for which an increase in the number/proportion and/or activity of senescent cells (relative to the non-diseased, healthy state) is positively associated with the severity of one or more symptoms of the disease/condition. A disease/condition in which cellular senescence is ‘pathologically-implicated’ may be a disease/condition for which an increase in the number/proportion and/or activity of senescent cells (relative to the non-diseased, healthy state) is a risk factor for the onset, development and/or progression of the disease/condition.

Diseases/conditions (e.g. age-related diseases/conditions) contemplated to be treated/presented in accordance with the present disclosure include e.g. geriatric syndromes, Alzheimer's disease, cancer, hyperlipidaemia, hypertriglyceridemia, hypercholesterolemia, steatosis (e.g. of the liver), non-alcoholic fatty liver disease (NAFLD), non-alcoholic fatty liver (NAFL) and non-alcoholic steatohepatitis (NASH), cardiovascular disease, hypertension (e.g. systolic, diastolic), heart failure with reduced or preserved ejection fraction, renal disease (e.g. chronic kidney disease), atherosclerosis, hypertension, maculopathy, age-related macular degeneration (AMD), cataracts, chronic obstructive pulmonary disease (COPD), arthritis, osteoarthritis, osteopenia, osteoporosis, Parkinson's disease, periodontitis, rheumatoid arthritis, diabetes mellitus, type II diabetes mellitus, chronic liver disease, sarcopenia, constipation, impotence, vaginal dryness, hair loss, skin disease and skin fragility.

Geriatric syndromes are conditions which are common in elderly patients, and include frailty, cognitive impairment, delirium, dementia, incontinence, hearing impairment, visual impairment, sarcopenia, metabolic syndrome, malnutrition, gait disturbance, falls and pressure ulcers.

Further exemplary diseases/conditions contemplated to be treated/presented in accordance with the present disclosure include frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

Where a disease/condition is described herein as “age-related”, reference is made to the disease/condition arising as a consequence of the age of the subject as distinct from other possible etiological causes. For example, “age-related steatosis” refers to the specific subtype of steatosis arising as a consequence of the aging process, which is distinct from steatosis arising e.g. as a consequence of diet.

In particular embodiments, the disease/condition to be treated/prevented in accordance with the present disclosure is selected from: osteoarthritis, osteopenia, osteoporosis, Parkinson's disease, periodontitis, frailty, cognitive impairment, delirium, dementia, incontinence, hearing impairment, visual impairment, malnutrition, gait disturbance, falls and pressure ulcers.

In some aspects and embodiments, the present disclosure contemplates the treatment/prevention of frailty.

Frailty may be determined in accordance with the phenotypic criteria established by Fried et al., J Gerontol A Biol Sci Med Sci (2001) 56(3):M146-56 (hereby incorporated by reference in its entirety), as subject having three or more of: low grip strength, low energy, slowed waking speed, low physical activity, and/or unintentional weight loss, which may in turn be defined in accordance with the frailty-defining criteria of the Women's Health and Aging Studies (WHAS) or the Cardiovascular Health Study (CHS) as summarised in Table 1 of Xue, Clin Geriatr Med (2011) February; 27(1): 1-15 (hereby incorporated by reference in its entirety).

In some aspects and embodiments, the present disclosure contemplates the treatment/prevention of an age-related change in body composition.

Age-related changes in body composition are described e.g. in Santanasto et al., J Gerontol A Biol Sci Med Sci. (2017) 72(4): 513-519 and St-Onge and Gallagher, Nutrition (2010) 26(2): 152-155, both of which are hereby incorporated by reference in their entirety. Age-related changes in body composition include: reduction in muscle mass (i.e. sarcopenia), reduction in bone mass (e.g. leading to osteoporosis), increase in fat mass, degeneration of cartilage (e.g. leading to osteoarthritis), changes in the kidney (e.g. leading to renal dysfunction (e.g. age-dependent deterioration in glomerular filtration rate)), changes in the organs of the respiratory system (e.g. the lungs, e.g. leading to chronic obstructive pulmonary disease (COPD)), changes in the organs of the digestive system (e.g. leading to constipation), changes in the bladder (e.g. leading to urinary incontinence), degeneration of teeth and/or gums (e.g. leading to periodontal disease), hair loss, skin fragility (e.g. leading to dryness and/or wrinkles), changes in the organs of the auditory system (e.g. leading to hearing loss), and changes to the reproductive organs (e.g. leading to impotence or vaginal dryness).

Age-related reduction in muscle mass may comprise a reduction in skeletal muscle mass. Skeletal muscle undergoes age-associated changes to the mitochondria, leading to the formation of inefficient mitochondria that release more reactive oxygen species (Johnson et al., Trends Endocrinol Metab. (2013) 24(5):247-56). Mitochondrial dysfunction is in turn thought to give rise to activation of skeletal muscle apoptosis, resulting in atrophy of skeletal muscle (Lenk et al. J Cachexia Sarcopenia Muscle. (2010) 1(1):9-21).

Age-related reduction in bone mass is described e.g. in Demontiero et al. Ther Adv Musculoskelet Dis. (2012) 4(2): 61-76. Underlying mechanisms include bone resorption by osteoclasts and insufficient formation of bone tissue by osteoblasts. Reduction in bone mass may result in osteoporosis, which is defined as defined as deterioration in bone mass and micro-architecture, with increasing risk to fragility fractures (Raisz and Rodan, Endocrinol Metab Clin North Am. (2003) 32(1):15-24).

Aging is often characterised by increased in body total fat mass independent from general and physiological fluctuations in weight and body mass index (BMI) (Zong et al., Obesity (2016) 24(11):2414-2421). In particular, accumulation of muscle fat, visceral fat and liver fat, in form of lipid droplets (LD), shows an age-dependent increase (Reinders et al., Curr Opin Clin Nutr Metab Care. (2017) 20(1):11-15).

In some embodiments, an age-related change in body composition in accordance with the present disclosure is selected from: age-related reduction in muscle mass, sarcopenia, age-related reduction in bone mass, osteoporosis, and age-related increase in fat mass. In some embodiments, an age-related change in body composition in accordance with the present disclosure is selected from: age-related reduction in muscle mass, sarcopenia and age-related increase in fat mass.

Aging is often associated with increases in serum lipid levels. Older adults display higher serum levels of triglycerides and cholesterol, and in some instances display hyperlipidaemia (e.g. hypertriglyceridemia, hypercholesterolemia or combined hyperlipidaemia (combination of hypertriglyceridemia and hypercholesterolemia). Hyperlipidaemia is in turn commonly associated e.g. with atherosclerosis and cardiovascular disease.

Hypertriglyceridemia is described e.g. in Berglund et al., J. Clin. Endocrinol. Metab. (2012) 97(9):2969-89, and is defined by blood triglyceride level ≥150 mg/dL (≥1.7 mmol/L). Hypercholesterolemia is described e.g. in Bhatnagar et al., BMJ (2008) 337:a993. The UK NHS defines hypercholesterolemia as blood total cholesterol level of ≥5 mmol/L or blood low-density lipoprotein (LDL) level of ≥3 mmol/L. The US NIH defines hypercholesterolemia as blood total cholesterol level of ≥240 mg/dL.

Aging is often associated with liver steatosis, e.g. as described in Nguyen et al., Cell Rep (2018) August 7; 24(6):1597-1609. Steatosis refers to the abnormal retention of lipid within a cell/tissue/organ. Steatosis may be macrovesicular or microvesicular, and commonly affects the liver. Age-related steatosis can lead to age-related non-alcoholic fatty liver disease (NAFLD). NAFLD is reviewed e.g. in Benedict and Zhang, World J Hepatol. (2017) 9(16): 715-732 and Albhaisi et al., Version 1. F1000Res. (2018) 7: F1000 Faculty Rev-720, both of which are hereby incorporated by reference in their entirety. NAFLD is characterised by steatosis of the liver, and in particular of hepatocytes. NAFLD includes non-alcoholic fatty liver (NAFL) and non-alcoholic steatohepatitis (NASH). NAFL is characterized by steatosis of the liver, involving greater than 5% of parenchyma, with no evidence of hepatocyte injury. NAFL may progress to NASH, which is steatosis combined with inflammation and/or fibrosis (steatohepatitis).

In particular embodiments, a disease/condition to be treated in accordance with the present disclosure may comprise or be characterised by one or more of the following: frailty, a reduction in muscle mass, an increase in fat mass, an increase in serum lipids (e.g. hyperlipidaemia), an increase in serum triglycerides (e.g. hypertriglyceridemia), an increase in serum cholesterol (e.g. hypercholesterolemia), an increase in liver triglycerides (e.g. steatosis of the liver), and a reduction in serum p-hydroxybutyrate. It will be appreciated that a reduction/increase is determined relative to the non-diseased state, or in the absence of the condition.

The therapeutic and prophylactic effects of the present disclosure are achieved through inhibition of IL-11-mediating signalling (i.e. antagonism of IL-11-mediated signalling), e.g. in a cell, tissue/organ/organ system/subject.

IL-11 is upregulated with age across all tissue in both male and female mice. It activates ERK to phosphorylate and inactivate LKB1 directly at LKB1(Serine325) and indirectly through P90RSK (LKB1(Serine428)). LKB1 has, until now with its demonstration by the inventors herein, been thought to be constitutively active and not regulated by phosphorylation. IL11-mediated ERK phosphorylation and inactivation of LKB1 leads to LKB1 dissociation from AMPK and AMPK is then dephosphorylated and inactivated. Inactive AMPK can no longer activate members of the TSC complex, which act to inhibit mTORC1. As such, mTORC1 becomes activated. Activated mTORC1 phosphorylates and activates P70S6K and RPS6 to stimulate protein synthesis and many other pro-aging pathways including inhibition of autophagy that impairs proteostasis. This schema is outlined in FIG. 16B. Thus, inhibition of IL-11-mediated signaling has wide-reaching and global therapeutic and prophylactic effects on ageing.

Without wishing to be bound by theory, IL-11 inhibits AMPK and activates mTOR via its upstream activity on LKB1, and the inventors have demonstrated that agents capable of inhibiting IL-11-mediated signaling have comparable or superior effects to putative anti-ageing agents metformin and rapamycin combined and exert a strong anti-ageing effect by increasing AMPK activity and decreasing mTOR activity.

Thus, in some embodiments, a disease/condition to be treated or prevented in accordance with the present disclosure may be associated with increased IL-11, decreased AMPK, and/or increased mTOR function, gene and/or protein expression, or activity in one or more affected tissues relative to a baseline healthy patient. In some embodiments, a disease/condition to be treated or prevented in accordance with the present disclosure may be associated with an IL-11 gain-of-function mutation in one or more affected tissues. Indeed, an “age-related” disease/condition or phenotype outlined herein may in some embodiments be associated with an IL-11 gain-of-function mutation in one or more affected tissues.

In some embodiments, a disease/condition to be treated or prevented in accordance with the present disclosure may be, or may be associated with, one or more Hallmark of Ageing. The “Hallmarks of Ageing”, as described in López-Otín et al. 2013 consist of: telomere attrition, genomic instability, mitochondrial dysfunction, cellular senescence, stem cell exhaustion, loss of proteostasis, deregulated nutrient sensing, epigenetic alterations, and altered intercellular communication. In some embodiments, the disease/condition to be treated or prevented in accordance with the present disclosure is, or is associated with, a Hallmark selected from deregulated nutrient sensing, loss of proteostasis and/or cellular senescence.

The utility of the therapeutic and prophylactic methods of the present disclosure extends to diseases/conditions that are caused or exacerbated by a disease/condition described hereinabove, or for which a disease/condition described hereinabove provides a poor prognosis.

In some embodiments, a disease/condition to be treated/prevented in accordance with the present disclosure may be characterised by an increase in the expression of IL-11 and/or IL-11Rα (i.e. gene and/or protein expression) in an organ/tissue/subject affected by the disease/condition e.g. as compared to normal organ/tissue/subject (i.e. in the absence of the disease/condition).

In some embodiments, the disease/condition may be associated with an upregulation of IL-11, e.g. an upregulation of IL-11 in cells or tissue in which the symptoms of the disease manifests or may occur, or upregulation of extracellular IL-11 or IL-11Rα.

Treatment may be effective to reduce/delay/prevent/reverse the development or progression of the disease/condition. Treatment may be effective to reduce/delay/prevent/reverse the worsening of one or more symptoms of the disease/condition. Treatment may be effective to improve one or more symptoms of the disease/condition. Treatment may be effective to reduce the severity of and/or reverse one or more symptoms of the disease/condition. Treatment may be effective to reverse the effects of the disease/condition.

Prevention may refer to prevention of development of the disease/condition, and/or prevention of worsening of the disease/condition, e.g. prevention of progression of the disease/condition, e.g. to a later/chronic stage.

Aspects and embodiments of the present disclosure relate to improving/increasing healthspan. The present disclosure provide methods comprising improving/increasing the healthspan of a subject, and articles (agents, compositions) for use in such methods.

As used herein, “healthspan” may be defined as the period of life spent free of age-related diseases/conditions. The healthspan of a given subject may therefore refer to the period during which the subject does not have an age-related disease/condition (e.g. an age-related disease described herein). A subject may be considered to not to have a given disease/condition until they are diagnosed with the relevant disease condition.

It will be appreciated that a subject having an improved/increased/extended healthspan remains free of an age-related disease/condition for a longer period as compared to a reference subject. In the context of the present disclosure, a subject administered with an agent capable of inhibiting IL-11-mediated signalling has an increased healthspan as compared to an equivalent, untreated subject.

In some embodiments of the present disclosure, a subject's healthspan may refer to the period during which the subject does not have (i.e. has not be diagnosed with) one of: a geriatric syndrome, Alzheimer's disease, cancer, hyperlipidaemia, hypertriglyceridemia, hypercholesterolemia, steatosis (e.g. of the liver), non-alcoholic fatty liver disease (NAFLD), non-alcoholic fatty liver (NAFL) and non-alcoholic steatohepatitis (NASH), cardiovascular disease, hypertension (e.g. systolic, diastolic), heart failure with reduced or preserved ejection fraction, renal disease (e.g. chronic kidney disease), atherosclerosis, hypertension, maculopathy, age-related macular degeneration (AMD), cataracts, chronic obstructive pulmonary disease (COPD), arthritis, osteoarthritis, osteopenia, osteoporosis, Parkinson's disease, periodontitis, rheumatoid arthritis, diabetes mellitus, type II diabetes mellitus, chronic liver disease, sarcopenia, constipation, impotence, vaginal dryness, hair loss, skin disease and skin fragility.

Alternatively, “healthspan” may be defined as the period of life during which a subject does not exhibit significant age-related deterioration in the function of a tissue, organ or organ system of the subject. The deterioration in function may arise as a consequence of an age-related disease/condition. A subject displaying deterioration in a function of a given tissue/organ/organ system may display a level of the relevant function which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level observed in comparable subjects not experiencing deterioration in the relevant function.

In accordance with various aspects of the present disclosure, the methods may comprise one or more of the following (e.g. in the context of treatment/prevention of a disease/condition described herein):
Reducing symptoms of frailty;
Increasing/maintaining lean mass;
Inhibiting age-related reduction in lean mass;
Reducing/maintaining fat mass;
Inhibiting age-related increase in fat mass;
Reducing age-related decline in metabolic function;
Reducing age-related hyperlipidemia;
Reducing age-related hypertriglyceridemia;
Reducing age-related hypercholesterolemia;
Reducing age-related steatosis (e.g. of the liver);
Increasing/maintaining fatty acid oxidation and/or ketone production;
Increasing/maintaining serum levels of β-hydroxybutyrate;
Reducing the number/size and/or severity of geropathological lesions
Inhibiting the development of geropathological lesions;
Reducing age-related fibrosis
Inhibiting the development of age-related fibrosis;
Inhibiting cellular senescence;
Increasing/maintaining cardiac function;
Inhibiting age-related deterioration of cardiac function;
Increasing/maintaining renal function;
Inhibiting age-related deterioration of renal function
Increasing/maintaining metabolic function;
Inhibiting age-related deterioration of metabolic function;
Increasing/maintaining liver function;
Inhibiting age-related deterioration of liver function;
Increasing/maintaining lung function;
Inhibiting age-related deterioration of lung function
Increasing/maintaining skin function; and
Inhibiting age-related deterioration of skin function.

### Administration

Administration of an agent capable of inhibiting IL-11-mediated signalling is preferably in a “therapeutically effective” or “prophylactically effective” amount, this being sufficient to show benefit to the subject.

The actual amount administered, and rate and time-course of administration, will depend on the nature and severity of the disease and the nature of the agent. Prescription of treatment, e.g. decisions on dosage etc., is within the responsibility of general practitioners and other medical doctors, and typically takes account of the disease/condition to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of the agent may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, 6, 8, 10 or 12 months. By way of example, doses may be given once every 7, 14, 21 or 28 days (plus or minus 3, 2, or 1 days).

In therapeutic applications, agents capable of inhibiting IL-11-mediated signalling are preferably formulated as a medicament or pharmaceutical together with one or more other pharmaceutically acceptable ingredients well-known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well-known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for suitable administration in accordance with the disease/condition to be treated, e.g. topical, parenteral, systemic, intravenous, intra-arterial, intramuscular, intrathecal, intraocular, intra-conjunctival, subcutaneous, oral, intra-dermal or transdermal routes of administration which may include injection. Injectable formulations may comprise the selected agent in a sterile or isotonic medium. Topical formulations may be provided as creams or lotions. The formulation and mode of administration may be selected according to the agent and disease to be treated.

### Detection of IL-11 and Receptors for IL-11

Some aspects and embodiments of the present disclosure concern detection of expression of IL-11 or a receptor for IL-11 (e.g. IL-11Rα, gp130, or a complex containing IL-11Rα and/or gp130) in a sample obtained from a subject.

In some aspects and embodiments the present disclosure concerns the upregulation of expression (over-expression) of IL-11 or a receptor for IL-11 (as a protein or oligonucleotide encoding the respective IL-11 or receptor for IL-11) and detection of such upregulation as an indicator of suitability for treatment with an agent capable of inhibiting the action of IL-11 or with an agent capable of preventing or reducing the expression of IL-11 or a receptor for IL-11.

Upregulated expression comprises expression at a level that is greater than would normally be expected for a cell or tissue of a given type. Upregulation may be determined by measuring the level of expression of the relevant factor in a cell or tissue. Comparison may be made between the level of expression in a cell or tissue sample from a subject and a reference level of expression for the relevant factor, e.g. a value or range of values representing a normal level of expression of the relevant factor for the same or corresponding cell or tissue type. In some embodiments reference levels may be determined by detecting expression of IL-11 or a receptor for IL-11 in a control sample, e.g. in corresponding cells or tissue from a healthy subject or from healthy tissue of the same subject. In some embodiments, reference levels may be obtained from a standard curve or data set.

Levels of expression may be quantitated for absolute comparison, or relative comparisons may be made.

In some embodiments, upregulation of IL-11 or a receptor for IL-11 (e.g. IL-11Rα, gp130, or a complex containing IL-11Rα and/or gp130) may be considered to be present when the level of expression in the test sample is at least 1.1 times that of a reference level. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times that of the reference level.

Expression levels may be determined by one of a number of known in vitro assay techniques, such as PCR based assays, in situ hybridisation assays, flow cytometry assays, immunological or immunohistochemical assays.

By way of example suitable techniques involve a method of detecting the level of IL-11 or a receptor for IL-11 in a sample by contacting the sample with an agent capable of binding IL-11 or a receptor for IL-11 and detecting the formation of a complex of the agent and IL-11 or receptor for IL-11. The agent may be any suitable binding molecule, e.g. an antibody, polypeptide, peptide, oligonucleotide, aptamer or small molecule, and may optionally be labelled to permit detection, e.g. visualisation, of the complexes formed. Suitable labels and means for their detection are well-known to those in the art and include fluorescent labels (e.g. fluorescein, rhodamine, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, Cy5), isotope markers, radioisotopes (e.g. 32P, 33P, 35S), chemiluminescence labels (e.g. acridinium ester, luminol, isoluminol), enzymes (e.g. peroxidase, alkaline phosphatase, glucose oxidase, beta-galactosidase, luciferase), antibodies, ligands and receptors. Detection techniques are well-known to those of skill in the art and can be selected to correspond with the labelling agent. Suitable techniques include PCR amplification of oligonucleotide tags, mass spectrometry, detection of fluorescence or colour, e.g. upon enzymatic conversion of a substrate by a reporter protein, or detection of radioactivity.

Assays may be configured to quantify the amount of IL-11 or receptor for IL-11 in a sample. Quantified amounts of IL-11 or receptor for IL-11 from a test sample may be compared with reference values, and the comparison used to determine whether the test sample contains an amount of IL-11 or receptor for IL-11 that is higher or lower than that of the reference value to a selected degree of statistical significance.

Quantification of detected IL-11 or receptor for IL-11 may be used to determine up- or down-regulation or amplification of genes encoding IL-11 or a receptor for IL-11. In cases where the test sample contains fibrotic cells, such up-regulation, down-regulation or amplification may be compared to a reference value to determine whether any statistically significant difference is present.

A sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject. Samples may be collected by known techniques, such as biopsy or needle aspirate. Samples may be stored and/or processed for subsequent determination of IL-11 expression levels.

Samples may be used to determine the upregulation of IL-11 or receptor for IL-11 in the subject from which the sample was taken.

In some preferred embodiments, a sample may be a tissue sample, e.g. biopsy, taken from a tissue/organ affected by a disease/condition described herein. A sample may contain cells.

A subject may be selected for therapy/prophylaxis in accordance with the present disclosure based on determination that the subject has an upregulated level of expression of IL-11 or of a receptor for IL-11 (e.g. IL-11Rα, gp130, or a complex containing IL-11Rα and/or gp130). Upregulated expression of IL-11 or of a receptor for IL-11 may serve as a marker of a disease/condition described herein suitable for treatment with an agent capable of inhibiting IL-11 mediated signalling.

Upregulation may be in a given tissue or in selected cells from a given tissue. Upregulation of expression of IL-11 or of a receptor for IL-11 may also be determined in a circulating fluid, e.g. blood, or in a blood-derived sample. Upregulation may be of extracellular IL-11 or IL-11Rα. In some embodiments expression may be locally or systemically upregulated.

Following selection, a subject may be administered with an agent capable of inhibiting IL-11 mediated signalling.

### Diagnosis and Prognosis

Detection of upregulation of expression of IL-11 or a receptor for IL-11 (e.g. IL-11Rα, gp130, or a complex containing IL-11Rα and/or gp130) may also be used in a method of diagnosing a disease/condition described herein, identifying a subject at risk of developing a disease/condition described herein, and in methods of prognosing or predicting a subject's response to treatment with an agent capable of inhibiting IL-11 mediated signalling.

“Developing”, “development” and other forms of “develop” may refer to the onset of a disorder/disease, or the continuation or progression of a disorder/disease.

In some embodiments a subject may be suspected of having or suffering from a disease/condition described herein, e.g. based on the presence of other symptoms indicative of the disease/condition in the subject's body or in selected cells/tissues of the subject's body, or may be considered at risk of developing the disease/condition, e.g. because of genetic predisposition or exposure to environmental conditions, known to be risk factors for the disease/condition. Determination of upregulation of expression of IL-11 or a receptor for IL-11 may confirm a diagnosis or suspected diagnosis, or may confirm that the subject is at risk of developing the disease/condition. The determination may also diagnose the disease/condition or predisposition as one suitable for treatment with an agent capable of inhibiting IL-11-mediated signalling.

As such, a method of providing a prognosis for a subject having, or suspected of having a disease/condition described herein may be provided, the method comprising determining whether the expression of IL-11 or a receptor for IL-11 is upregulated in a sample obtained from the subject and, based on the determination, providing a prognosis for treatment of the subject with an agent capable of inhibiting IL-11-mediated signalling.

In some aspects, methods of diagnosis or methods of prognosing or predicting a subject's response to treatment with an agent capable of inhibiting IL-11-mediated signalling may not require determination of the expression of IL-11 or a receptor for IL-11, but may be based on determining genetic factors in the subject that are predictive of upregulation of expression or activity. Such genetic factors may include the determination of genetic mutations, single nucleotide polymorphisms (SNPs) or gene amplification in IL-11, IL-11Rα and/or gp130 which are correlated with and/or predictive of upregulation of expression or activity and/or IL-11 mediated signalling. The use of genetic factors to predict predisposition to a disease state or response to treatment is known in the art, e.g. see Peter Stärkel Gut 2008; 57:440-442; Wright et al., Mol. Cell. Biol. March 2010 vol. 30 no. 6 1411-1420.

Genetic factors may be assayed by methods known to those of ordinary skill in the art, including PCR based assays, e.g. quantitative PCR, competitive PCR. By determining the presence of genetic factors, e.g. in a sample obtained from a subject, a diagnosis may be confirmed, and/or a subject may be classified as being at risk of developing a disease/condition described herein, and/or a subject may be identified as being suitable for treatment with an agent capable of inhibiting IL-11 mediated signalling.

Some methods may comprise determination of the presence of one or more SNPs linked to secretion of IL-11 or susceptibility to development of a disease/condition described herein. SNPs are usually bi-allelic and therefore can be readily determined using one of a number of conventional assays known to those of skill in the art (e.g. see Anthony J. Brookes. The essence of SNPs. Gene Volume 234, Issue 2, 8 Jul. 1999, 177-186; Fan et al., Highly Parallel SNP Genotyping. Cold Spring Harb Symp Quant Biol 2003. 68: 69-78; Matsuzaki et al., Parallel Genotyping of Over 10,000 SNPs using a one-primer assay on a high-density oligonucleotide array. Genome Res. 2004. 14: 414-425).

The methods may comprise determining which SNP allele is present in a sample obtained from a subject. In some embodiments determining the presence of the minor allele may be associated with increased IL-11 secretion or susceptibility to development of a disease/condition described herein.

Accordingly, in one aspect of the present disclosure a method for screening a subject is provided, the method comprising:
obtaining a nucleic acid sample from the subject;
determining which allele is present in the sample at the polymorphic nucleotide position of one or more of the SNPs listed in FIG. 33, FIG. 34, or FIG. 35 of WO 2017/103108 A1 (incorporated by reference herein), or a SNP in linkage disequilibrium with one of the listed SNPs with an r²≥0.8.

The determining step may comprise determining whether the minor allele is present in the sample at the selected polymorphic nucleotide position. It may comprise determining whether 0, 1 or 2 minor alleles are present.

The screening method may be, or form part of, a method for determining susceptibility of the subject to development of a disease/condition described herein, or a method of diagnosis or prognosis as described herein.

The method may further comprise the step of identifying the subject as having susceptibility to, or an increased risk of, developing a disease/condition described herein, e.g. if the subject is determined to have a minor allele at the polymorphic nucleotide position. The method may further comprise the step of selecting the subject for treatment with an agent capable of inhibiting IL-11 mediated signalling and/or administering an agent capable of inhibiting IL-11 mediated signalling to the subject in order to provide a treatment for a disease/condition described herein in the subject or to prevent development or progression of the disease/condition in the subject.

In some embodiments, a method of diagnosing a disease/condition described herein, identifying a subject at risk of developing a disease/condition described herein, and methods of prognosing or predicting a subject's response to treatment with an agent capable of inhibiting IL-11 mediated signalling employs an indicator that is not detection of upregulation of expression of IL-11 or a receptor for IL-11, or genetic factors.

In some embodiments, a method of diagnosing a disease/condition described herein, identifying a subject at risk of developing the disease/condition, and methods of prognosing or predicting a subject's response to treatment with an agent capable of inhibiting IL-11 mediated signalling is based on detecting, measuring and/or identifying one or more indicators of the disease/condition.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body. The sample obtained from a subject may be of any kind, as described herein above.

Other diagnostic or prognostic tests may be used in conjunction with those described here to enhance the accuracy of the diagnosis or prognosis or to confirm a result obtained by using the tests described here.

### Subjects

Subjects may be animal or human. Subjects are preferably mammalian, more preferably human. The subject may be a non-human mammal, but is more preferably human. The subject may be male or female. The subject may be a patient. The patient may have a disease/condition described herein.

The subject may be a subject in need of therapeutic/prophylactic intervention in accordance with the present disclosure. The subject may be a subject that would benefit from therapeutic/prophylactic intervention in accordance with the present disclosure.

The subject may have been diagnosed with a disease/condition described herein requiring treatment, may be suspected of having such a disease/condition, or may be at risk from developing such a disease/condition.

In embodiments according to the present disclosure the subject is preferably a human subject. In embodiments according to the present disclosure, a subject may be selected for treatment according to the methods based on characterisation for certain markers of a disease/condition described herein.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-21 7), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human IL-11 (UniProt | MNCVCRLVLVVLSLWPDTAVAPGPPPGPPRVSPDPRAELDSTVLLTRSLLADTRQL |
| | P20809) | AAQLRDKFPADGDHNLDSLPTLAMSAGALGALQLPGVLTRLRADLLSYLRHVQWLR |
| | | RAGGSSLKTLEPELGTLQARLDRLLRRLQLLMSRLALPQPPPDPPAPPLAPPSSAW |
| | | GGIRAAHAILGGLHLTLDWAVRGLLLLKTRL |
| 2 | Human gp130 | MLTLQTWLVQALFIFLTTESTGELLDPCGYISPESPVVQLHSNFTAVCVLKEKCMD |
| | (UniProt P40189-1) | YFHVNANYIVWKTNHFTIPKEQYTIINRTASSVTFTDIASLNIQLTCNILTFGQLE |
| | | QNVYGITIISGLPPEKPKNLSCIVNEGKKMRCEWDGGRETHLETNFTLKSEWATHK |
| | | FADCKAKRDTPTSCTVDYSTVYFVNIEVWVEAENALGKVTSDHINFDPVYKVKPNP |
| | | PHNLSVINSEELSSILKLTWTNPSIKSVIILKYNIQYRTKDASTWSQIPPEDTAST |
| | | RSSFTVQDLKPFTEYVFRIRCMKEDGKGYWSDWSEEASGITYEDRPSKAPSFWYKI |
| | | DPSHTQGYRTVQLVWKTLPPFEANGKILDYEVTLTRWKSHLQNYTVNATKLTVNLT |
| | | NDRYLATLTVRNLVGKSDAAVLTIPACDFQATHPVMDLKAFPKDNMLWVEWTTPRE |
| | | SVKKYILEWCVLSDKAPCITDWQQEDGTVHRTYLRGNLAESKCYLITVTPVYADGP |
| | | GSPESIKAYLKQAPPSKGPTVRTKKVGKNEAVLEWDQLPVDVQNGFIRNYTIFYRT |
| | | IIGNETAVNVDSSHTEYTLSSLTSDTLYMVRMAAYTDEGGKDGPEFTFTTPKFAQG |
| | | EIEAIVVPVCLAFLLTTLLGVLFCFNKRDLIKKHIWPNVPDPSKSHIAQWSPHTPP |
| | | RHNFNSKDQMYSDGNFTDVSVVEIEANDKKPFPEDLKSLDLFKKEKINTEGHSSGI |
| | | GGSSCMSSSRPSISSSDENESSQNTSSTVQYSTVVHSGYRHQVPSVQVFSRSESTQ |
| | | PLLDSEERPEDLQLVDHVDGGDGILPRQQYFKQNCSQHESSPDISHFERSKQVSSV |
| | | NEEDFVRLKQQISDHISQSCGSGQMKMFQEVSAADAFGPGTEGQVERFETVGMEAA |
| | | TDEGMPKSYLPQTVRQGGYMPQ |
| 3 | Human IL11RA | MSSSCSGLSRVLVAVATALVSASSPCPQAWGPPGVQYGQPGRSVKLCCPGVTAGDP |
| | (UniProt Q14626) | VSWFRDGEPKLLQGPDSGLGHELVLAQADSTDEGTYICQTLDGALGGTVTLQLGYP |
| | | PARPVVSCQAADYENFSCTWSPSQISGLPTRYLTSYRKKTVLGADSQRRSPSTGPW |
| | | PCPQDPLGAARCVVHGAEFWSQYRINVTEVNPLGASTRLLDVSLQSILRPDPPQGL |
| | | RVESVPGYPRRLRASWTYPASWPCQPHFLLKFRLQYRPAQHPAWSTVEPAGLEEVI |
| | | TDAVAGLPHAVRVSARDFLDAGTWSTWSPEAWGTPSTGTIPKEIPAWGQLHTQPEV |
| | | EPQVDSPAPPRPSLQPHPRLLDHRDSVEQVAVLASLGILSFLGLVAGALALGLWLR |
| | | LRRGGKDGSPKPGFLASVIPVDRRPGAPNL |
| 4 | siRNA target IL-11 | CCTTCCAAAGCCAGATCTT |
| 5 | siRNA target IL-11 | GCCTGGGCAGGAACATATA |
| 6 | siRNA target IL-11 | CCTGGGCAGGAACATATAT |
| 7 | siRNA target IL-11 | GGTTCATTATGGCTGTGTT |
| 8 | siRNA target IL-11Rα | GGACCATACCAAAGGAGAT |
| 9 | siRNA target IL-11Rα | GCGTCTTTGGGAATCCTTT |
| 10 | siRNA target IL-11Rα | GCAGGACAGTAGATCCCT |
| 11 | siRNA target IL-11Rα | GCTCAAGGAACGTGTGTAA |
| 12 | siRNA to IL-11 | CCUUCCAAAGCCAGAUCUUdTdT-AAGAUCUGGCUUUGGAAGGdTdT |
| | (NM_000641.3) | |
| 13 | siRNA to IL-11 | GCCUGGGCAGGAACAUAUAdTdT-UAUAUGUUCCUGCCCAGGCdTdT |
| | (NM_000641.3) | |
| 14 | siRNA to IL-11 | CCUGGGCAGGAACAUAUAUdTdT-AUAUAUGUUCCUGCCCAGGdTdT |
| | (NM_000641.3) | |
| 15 | siRNA to IL-11 | GGUUCAUUAUGGCUGUGUUdTdT-AACACAGCCAUAAUGAACCdTdT |
| | (NM_000641.3) | |
| 16 | siRNA to IL-11Rα | GGACCAUACCAAAGGAGAUdTdT-AUCUCCUUUGGUAUGGUCCdTdT |
| | (U32324.1) | |
| 17 | siRNA to IL-11Rα | GCGUCUUUGGGAAUCCUUUdTdT-AAAGGAUUCCCAAAGACGCdTdT |
| | (U32324.1) | |
| 18 | siRNA to IL-11Rα | GCAGGACAGUAGAUCCCUAdTdT-UAGGGAUCUACUGUCCUGCdTdT |
| | (U32324.1) | |
| 19 | siRNA to IL-11Rα | GCUCAAGGAACGUGUGUAAdTdT-UUACACACGUUCCUUGAGCdTdT |
| | (U32324.1) | |
| 20 | 20 amino acid linker | GPAGQSGGGGGSGGGSGGGSV |
| 21 | Hyper IL-11 (IL- | MSSSCSGLSRVLVAVATALVSASSPCPQAWGPPGVQYGQPGRSVKLCCPGVTAGDP |
| | 11RA:IL-11 fusion) | VSWFRDGEPKLLQGPDSGLGHELVLAQADSTDEGTYICQTLDGALGGTVTLQLGYP |
| | | PARPVVSCQAADYENFSCTWSPSQISGLPTRYLTSYRKKTVLGADSQRRSPSTGPW |
| | | PCPQDPLGAARCVVHGAEFWSQYRINVTEVNPLGASTRLLDVSLQSILRPDPPQGL |
| | | RVESVPGYPRRLRASWTYPASWPCQPHFLLKFRLQYRPAQHPAWSTVEPAGLEEVI |
| | | TDAVAGLPHAVRVSARDFLDAGTWSTWSPEAWGTPSTGPAGQSGGGGGSGGGSGGG |
| | | SVPGPPPGPPRVSPDPRAELDSTVLLTRSLLADTRQLAAQLRDKFPADGDHNLDSL |
| | | PTLAMSAGALGALQLPGVLTRLRADLLSYLRHVQWLRRAGGSSLKTLEPELGTLQA |
| | | RLDRLLRRLQLLMSRLALPQPPPDPPAPPLAPPSSAWGGIRAAHAILGGLHLTLDW |
| | | AVRGLLLLKTRL |
| 22 | Enx203 VH | EVQLQQSGPELVKPGASVKIPCKASGYTFTDYNMDWVKQSHGKSLEWIGDINPHNG |
| | | GPIYNQKFTGKATLTVDKSSSTAYMELRSLTSEDTAVYYCARGELGHWYFDVWGTG |
| | | TTVTVSS |
| 23 | Enx203 VL | DIVLTQSPASLAVSLGQRATISCRASKSVSTSGYSYIHWYQQKPGQPPKLLIYLAS |
| | | NLDSGVPARFSGSGSGTDFTLNIHPVEEEDAATYYCQHSRDLPPTFGGGTKLEIK |
| 24 | Enx209 VH | QVQLQQPGAELVRPGSSVKLSCKASGYTFTNYWMHWLKQRPVQGLEWIGNIGPSDS |
| | | KTHYNQKFKDKATLTVDKSSSTAYMQLNSLTSEDSAVYYCARGDYVLFTYWGQGTL |
| | | VTVSA |
| 25 | Enx209 VL | DIVLTQSPATLSLSPGERATLSCRASQSISNNLHWYQQKSHEAPRLLIKYASQSIS |
| | | GIPARFSGSGSGTDFTLSFSSLETEDFAVYFCQQSYSWPLTFGQGTKLEIK |
| 26 | Enx108A VH | QVQLVQSGGGVVQPGRSLRLSCAASGFTFSSYGMHWVRQAPGKGLEWVAVISYDGS |
| | | NKYYADSVKGRFTISRDNSKNTLYLQMNSLRAEDTAVYYCAKIGATDPLDYWGQGT |
| | | LVTVSS |
| 27 | Enx108A VL | QSALTQPRSVSGSPGQSVTLSCTGTSSDVGGYNYVSWYQHYPGKAPKLMIFDVNER |
| | | SSGVPDRFSGSKSGNTASLTISGLQAEDEADYYCASYAGRYTWMFGGGTKVTVLG |
| 28 | Enx108A hIgG4 | QVQLVQSGGGVVQPGRSLRLSCAASGFTFSSYGMHWVRQAPGKGLEWVAVISYDGS |
| | (L248E, S241P) HC | NKYYADSVKGRFTISRDNSKNTLYLQMNSLRAEDTAVYYCAKIGATDPLDYWGQGT |
| | | LVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPC |
| | | PPCPAPEFEGGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLPSSIEKTISK |
| | | AKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| 29 | Enx108A lambda LC | QSALTQPRSVSGSPGQSVTLSCTGTSSDVGGYNYVSWYQHYPGKAPKLMIFDVNER |
| | | SSGVPDRFSGSKSGNTASLTISGLQAEDEADYYCASYAGRYTWMFGGGTKVTVLGQ |
| | | PKAAPSVTLFPPSSEELQANKATLVCLISDFYPGAVTVAWKADSSPVKAGVETTTP |
| | | SKQSNNKYAASSYLSLTPEQWKSHRSYSCQVTHEGSTVEKTVAPTECS |
| 30 | hEnx203 VH | EVQLVQSGAEVKKPGASVKISCKASGYTFTDYNMDWVKQAPGQRLEWIGDINPHNG |
| | | GPIYNQKFTGRATLTVDKSASTAYMELSSLRSEDTAVYYCARGELGHWYFDVWGQG |
| | | TTVTVSS |
| 31 | hEnx203 VL | DIVLTQSPASLALSPGERATLSCRASKSVSTSGYSYIHWYQQKPGQAPRLLIYLAS |
| | | NLDSGVPARFSGSGSGTDFTLTISSLEEEDFATYYCQHSRDLPPTFGQGTKLEIK |
| 32 | hEnx209 VH | QVQLVQSGAEVKKPGASVKVSCKASGYTFTNYWMHWLRQRPGQGLEWIGNIGPSDS |
| | | KTHYNQKFKDRVTMTVDKSTSTAYMELSSLRSEDTAVYYCARGDYVLFTYWGQGTL |
| | | VTVSS |
| 33 | hEnx209 VL | DIVLTQSPATLSLSPGERATLSCRASQSISNNLHWYQQKPGQAPRLLIKYASQSIS |
| | | GIPARFSGSGSGTDFTLTISSLEPEDFAVYYCQQSYSWPLTFGQGTKLEIK |
| 34 | Enx108A VH CDR1 | SYGMH |
| 35 | Enx108A VH CDR2 | VISYDGSNKYYADSVKG |
| 36 | Enx108A VH CDR3 | IGATDPLDY |
| 37 | Enx108A VL CDR1 | TGTSSDVGGYNYVS |
| 38 | Enx108A VL CDR2 | DVNERSS |
| 39 | Enx108A VL CDR3 | ASYAGRYTWM |
| 40 | Enx203, hEnx203 VH | DYNMD |
| | CDR1 | |
| 41 | Enx203, hEnx203 VH | DINPHNGGPIYNQKFTG |
| | CDR2 | |
| 42 | Enx203, hEnx203 VH | GELGHWYFDV |
| | CDR3 | |
| 43 | Enx203, hEnx203 VL | RASKSVSTSGYSYIH |
| | CDR1 | |
| 44 | Enx203, hEnx203 VL | LASNLDS |
| | CDR2 | |
| 45 | Enx203, hEnx203 VL | QHSRDLPPT |
| | CDR3 | |
| 46 | Enx209, hEnx209 VH | NYWMH |
| | CDR1 | |
| 47 | Enx209, hEnx209 VH | NIGPSDSKTHYNQKFKD |
| | CDR2 | |
| 48 | Enx209, hEnx209 VH | GDYVLFTY |
| | CDR3 | |
| 49 | Enx209, hEnx209 VL | RASQSISNNLH |
| | CDR1 | |
| 50 | Enx209, hEnx209 VL | YASQSIS |
| | CDR2 | |
| 51 | Enx209, hEnx209 VL | QQSYSWPLT |
| | CDR3 | |
| 52 | Human IGHG1 | ASTKGPSVFPLAPSSKSTSGGTAALGCLVKDYFPEPVTVSWNSGALTSGVHTFPAV |
| | constant (K214R, | LQSSGLYSLSSVVTVPSSSLGTQTYICNVNHKPSNTKVDKRVEPKSCDKTHTCPPC |
| | D356E, L358M) | PAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 53 | Human IGHG4 | ASTKGPSVFPLAPCSRSTSESTAALGCLVKDYFPEPVTVSWNSGALTSGVHTFPAV |
| | constant (L248E, | LQSSGLYSLSSVVTVPSSSLGTKTYTCNVDHKPSNTKVDKRVESKYGPPCPPCPAP |
| | S241P) | EFEGGPSVFLFPPKPKDTLMISRTPEVTCVVVDVSQEDPEVQFNWYVDGVEVHNAK |
| | | TKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLGK |
| 54 | Human IGKC | RTVAAPSVFIFPPSDEQLKSGTASVVCLLNNFYPREAKVQWKVDNALQSGNSQESV |
| | constant | TEQDSKDSTYSLSSTLTLSKADYEKHKVYACEVTHQGLSSPVTKSFNRGEC |
| 55 | Human IGLC2 | GQPKAAPSVTLFPPSSEELQANKATLVCLISDFYPGAVTVAWKADSSPVKAGVETT |
| | constant | TPSKQSNNKYAASSYLSLTPEQWKSHRSYSCQVTHEGSTVEKTVAPTECS |
| 56 | hEnx203 hIgG1 HC | EVQLVQSGAEVKKPGASVKISCKASGYTFTDYNMDWVKQAPGQRLEWIGDINPHNG |
| | | GPIYNQKFTGRATLTVDKSASTAYMELSSLRSEDTAVYYCARGELGHWYFDVWGQG |
| | | TTVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKSLSLSPG |
| | | K |
| 57 | hEnx203 kappa LC | DIVLTQSPASLALSPGERATLSCRASKSVSTSGYSYIHWYQQKPGQAPRLLIYLAS |
| | | NLDSGVPARFSGSGSGTDFTLTISSLEEEDFATYYCQHSRDLPPTFGQGTKLEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 58 | hEnx209 hIgG4 | QVQLVQSGAEVKKPGASVKVSCKASGYTFTNYWMHWLRQRPGQGLEWIGNIGPSDS |
| | (L248E, S241P) HC | KTHYNQKFKDRVTMTVDKSTSTAYMELSSLRSEDTAVYYCARGDYVLFTYWGQGTL |
| | | VTVSSASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESKYGPPCP |
| | | PCPAPEFEGGPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPSSIEKTISKA |
| | | KGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 59 | hEnx209 kappa LC | DIVLTQSPATLSLSPGERATLSCRASQSISNNLHWYQQKPGQAPRLLIKYASQSIS |
| | | GIPARFSGSGSGTDFTLTISSLEPEDFAVYYCQQSYSWPLTFGQGTKLEIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |

### Numbered Paragraphs

The following numbered paragraphs (paras) provide further statements of features and combinations of features which are contemplated in connection with the present invention:
1. An agent capable of inhibiting interleukin 11 (IL-11)-mediated signalling for use in a method of treating or preventing an age-related disease/condition.
2. Use of an agent capable of inhibiting interleukin 11 (IL-11)-mediated signalling in the manufacture of a medicament for use in a method of treating or preventing an age-related disease/condition.
3. A method of treating or preventing an age-related disease/condition, the method comprising administering a therapeutically or prophylactically effective amount of an agent capable of inhibiting interleukin 11 (IL-11)-mediated signalling to a subject.
4. An agent capable of inhibiting interleukin 11 (IL-11)-mediated signalling for use in a method of inhibiting cellular senescence and/or inhibiting the activity of senescent cells in a subject in need thereof.
5. Use of an agent capable of inhibiting interleukin 11 (IL-11)-mediated signalling in the manufacture of a medicament for use in a method of inhibiting cellular senescence and/or inhibiting the activity of senescent cells in a subject in need thereof.
6. A method of inhibiting cellular senescence and/or inhibiting the activity of senescent cells in a subject in need thereof, the method comprising administering a therapeutically or prophylactically effective amount of an agent capable of inhibiting interleukin 11 (IL-11)-mediated signalling to the subject in need thereof.
7. The agent for use according to para 4, the use according to para 5, or the method according to para 6, wherein the subject is a subject having a disease or condition characterised by cellular senescence.
8. The agent for use, the use, or the method according to para 7, wherein the disease or condition characterised by cellular senescence is an age-related disease or condition.
9. The agent for use, the use, or the method according to para 7 or para 8, wherein the disease or condition characterised by cellular senescence is selected from: a geriatric syndrome, Alzheimer's disease, cardiovascular disease, atherosclerosis, hypertension, maculopathy, chronic obstructive pulmonary disease (COPD), osteoarthritis, osteopenia, osteoporosis, Parkinson's disease, periodontitis, rheumatoid arthritis, diabetes mellitus or sarcopenia.
10. An agent capable of inhibiting interleukin 11 (IL-11)-mediated signalling for use in a method of treating or preventing an age-related disease or condition selected from: a geriatric syndrome, Alzheimer's disease, cancer, hyperlipidaemia, hypertriglyceridemia, hypercholesterolemia, steatosis (e.g. of the liver), non-alcoholic fatty liver disease (NAFLD), non-alcoholic fatty liver (NAFL) and non-alcoholic steatohepatitis (NASH), cardiovascular disease, hypertension (e.g. systolic, diastolic), heart failure with reduced or preserved ejection fraction, renal disease (e.g. chronic kidney disease), atherosclerosis, hypertension, maculopathy, age-related macular degeneration (AMD), cataracts, chronic obstructive pulmonary disease (COPD), arthritis, osteoarthritis, osteopenia, osteoporosis, Parkinson's disease, periodontitis, rheumatoid arthritis, diabetes mellitus, type II diabetes mellitus, chronic liver disease, sarcopenia, constipation, impotence, vaginal dryness, hair loss, skin disease and skin fragility.
11. Use of an agent capable of inhibiting interleukin 11 (IL-11)-mediated signalling in the manufacture of a medicament for use in a method of treating or preventing an age-related disease or condition selected from: a geriatric syndrome, Alzheimer's disease, cancer, hyperlipidaemia, hypertriglyceridemia, hypercholesterolemia, steatosis (e.g. of the liver), non-alcoholic fatty liver disease (NAFLD), non-alcoholic fatty liver (NAFL) and non-alcoholic steatohepatitis (NASH), cardiovascular disease, hypertension (e.g. systolic, diastolic), heart failure with reduced or preserved ejection fraction, renal disease (e.g. chronic kidney disease), atherosclerosis, hypertension, maculopathy, age-related macular degeneration (AMD), cataracts, chronic obstructive pulmonary disease (COPD), arthritis, osteoarthritis, osteopenia, osteoporosis, Parkinson's disease, periodontitis, rheumatoid arthritis, diabetes mellitus, type II diabetes mellitus, chronic liver disease, sarcopenia, constipation, impotence, vaginal dryness, hair loss, skin disease and skin fragility.
12. A method of treating or preventing an age-related disease or condition, comprising administering a therapeutically or prophylactically effective amount of an agent capable of inhibiting interleukin 11 (IL-11)-mediated signalling to a subject having an age-related disease or condition selected from: a geriatric syndrome, Alzheimer's disease, cancer, hyperlipidaemia, hypertriglyceridemia, hypercholesterolemia, steatosis (e.g. of the liver), non-alcoholic fatty liver disease (NAFLD), non-alcoholic fatty liver (NAFL) and non-alcoholic steatohepatitis (NASH), cardiovascular disease, hypertension (e.g. systolic, diastolic), heart failure with reduced or preserved ejection fraction, renal disease (e.g. chronic kidney disease), atherosclerosis, hypertension, maculopathy, age-related macular degeneration (AMD), cataracts, chronic obstructive pulmonary disease (COPD), arthritis, osteoarthritis, osteopenia, osteoporosis, Parkinson's disease, periodontitis, rheumatoid arthritis, diabetes mellitus, type II diabetes mellitus, chronic liver disease, sarcopenia, constipation, impotence, vaginal dryness, hair loss, skin disease and skin fragility.
13. An agent capable of inhibiting interleukin 11 (IL-11)-mediated signalling for use in a method of treating or preventing frailty.
14. Use of an agent capable of inhibiting interleukin 11 (IL-11)-mediated signalling in the manufacture of a medicament for use in a method of treating or preventing frailty.
15. A method of treating or preventing frailty, comprising administering a therapeutically or prophylactically effective amount of an agent capable of inhibiting interleukin 11 (IL-11)-mediated signalling to a subject.
16. An agent capable of inhibiting interleukin 11 (IL-11)-mediated signalling for use in a method of treating or preventing an age-related change in body composition.
17. Use of an agent capable of inhibiting interleukin 11 (IL-11)-mediated signalling in the manufacture of a medicament for use in a method of treating or preventing an age-related change in body composition.
18. A method of treating or preventing an age-related change in body composition, comprising administering a therapeutically or prophylactically effective amount of an agent capable of inhibiting interleukin 11 (IL-11)-mediated signalling to a subject.
19. The agent for use, the use, or the method according to any one of paras 16 to 18, wherein the age-related change in body composition is selected from: reduction in muscle mass, reduction in bone mass, increase in fat mass, degeneration of cartilage, changes in the kidney, changes in the organs of the respiratory system, changes in the organs of the digestive system, changes in the bladder, degeneration of teeth and/or gums, hair loss, skin fragility, changes in the organs of the auditory system, and changes to the reproductive organs.
20. An agent capable of inhibiting interleukin 11 (IL-11)-mediated signalling for use in a method of increasing the healthspan of a subject.
21. Use of an agent capable of inhibiting interleukin 11 (IL-11)-mediated signalling in the manufacture of a medicament for use in a method of increasing the healthspan of a subject.
22. A method of increasing the healthspan of a subject, comprising administering a therapeutically or prophylactically effective amount of an agent capable of inhibiting interleukin 11 (IL-11)-mediated signalling to the subject.
23. The agent for use, the use, or the method according to any one of paras 1 to 22, wherein the agent is selected from the group consisting of: an antibody or an antigen-binding fragment thereof, a polypeptide, a peptide, a nucleic acid, an oligonucleotide, an aptamer or a small molecule.
24. The agent for use, the use, or the method according to any one of paras 1 to 23, wherein the agent is an agent capable of preventing or reducing the binding of interleukin 11 (IL-11) to a receptor for interleukin 11 (IL-11R).
25. The agent for use, the use or the method according to any one of paras 1 to 24, wherein the agent is capable of binding to interleukin 11 (IL-11) or a receptor for interleukin 11 (IL-11R).
26. The agent for use, the use or the method according to any one of paras 1 to 25, wherein the agent is an antibody or an antigen-binding fragment thereof.
27. The agent for use, the use or the method according to any one of paras 1 to 26, wherein the agent is an anti-IL-11 antibody antagonist of IL-11-mediated signalling, or an antigen-binding fragment thereof.
28. The agent for use, the use or the method according to any one of paras 1 to 27, wherein the antibody or antigen-binding fragment comprises:
   (i) a heavy chain variable (VH) region incorporating the following CDRs:
      HC-CDR1 having the amino acid sequence of SEQ ID NO:34
      HC-CDR2 having the amino acid sequence of SEQ ID NO:35
      HC-CDR3 having the amino acid sequence of SEQ ID NO:36; and
   (ii) a light chain variable (VL) region incorporating the following CDRs:
      LC-CDR1 having the amino acid sequence of SEQ ID NO:37
      LC-CDR2 having the amino acid sequence of SEQ ID NO:38
      LC-CDR3 having the amino acid sequence of SEQ ID NO:39.
29. The agent for use, the use or the method according to any one of paras 1 to 27, wherein the antibody or antigen-binding fragment comprises:
   (i) a heavy chain variable (VH) region incorporating the following CDRs:
      HC-CDR1 having the amino acid sequence of SEQ ID NO:40
      HC-CDR2 having the amino acid sequence of SEQ ID NO:41
      HC-CDR3 having the amino acid sequence of SEQ ID NO:42; and
   (ii) a light chain variable (VL) region incorporating the following CDRs:
      LC-CDR1 having the amino acid sequence of SEQ ID NO:43
      LC-CDR2 having the amino acid sequence of SEQ ID NO:44
      LC-CDR3 having the amino acid sequence of SEQ ID NO:45.
30. The agent for use, the use or the method according to any one of paras 1 to 26, wherein the agent is an anti-IL-11Rα antibody antagonist of IL-11-mediated signalling, or an antigen-binding fragment thereof.
31. The agent for use, the use, or the method according to any one of paras 1 to 26 or para 30, wherein the antibody or antigen-binding fragment comprises:
   (i) a heavy chain variable (VH) region incorporating the following CDRs:
      HC-CDR1 having the amino acid sequence of SEQ ID NO:46
      HC-CDR2 having the amino acid sequence of SEQ ID NO:47
      HC-CDR3 having the amino acid sequence of SEQ ID NO:48; and
   (ii) a light chain variable (VL) region incorporating the following CDRs:
      LC-CDR1 having the amino acid sequence of SEQ ID NO:49
      LC-CDR2 having the amino acid sequence of SEQ ID NO:50
      LC-CDR3 having the amino acid sequence of SEQ ID NO:51.
32. The agent for use, the use or the method according to any one of paras 1 to 25, wherein the agent is a decoy receptor for IL-11.
33. The agent for use, the use or the method according to para 32, wherein the decoy receptor for IL-11 comprises: (i) an amino acid sequence corresponding to the cytokine binding module of gp130 and (ii) an amino acid sequence corresponding to the cytokine binding module of IL-11Rα.
34. The agent for use, the use or the method according to any one of paras 1 to 25, wherein the agent is a competitive inhibitor of IL-11.
35. The agent for use, the use or the method according to para 34, wherein the agent is an IL-11 mutein.
36. The agent for use, the use or the method according to para 35, wherein the IL-11 mutein is W147A.
37. The agent for use, the use or the method according to any one of paras 1 to 23, wherein the agent is capable of preventing or reducing the expression of interleukin 11 (IL-11) or a receptor for interleukin 11 (IL-11R).
38. The agent for use, the use or the method according to para 37, wherein the agent is an antisense oligonucleotide capable of preventing or reducing the expression of IL-11.
39. The agent for use, the use or the method according to para 38, wherein the antisense oligonucleotide capable of preventing or reducing the expression of IL-11 is siRNA targeted to IL11 comprising the sequence of SEQ ID NO:12, 13, 14 or 15.
40. The agent for use, the use or the method according to para 37, wherein the agent is an antisense oligonucleotide capable of preventing or reducing the expression of IL-11Rα.
41. The agent for use, the use or the method according to para 40, wherein the antisense oligonucleotide capable of preventing or reducing the expression of IL-11Rα is siRNA targeted to IL11RA comprising the sequence of SEQ ID NO:16, 17, 18 or 19.
42. The agent for use, the use or the method according to any one of paras 1 to 41, wherein the method comprises administering the agent to a subject in which expression of interleukin 11 (IL-11) or a receptor for IL-11 (IL-11R) is upregulated.

The present disclosure includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the present disclosure in diverse forms thereof.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Methods disclosed herein may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. In some embodiments, methods performed in vivo may be performed on non-human animals. “Ex vivo” refers to something present or taking place outside an organism, e.g. outside the human or animal body, which may be on tissue (e.g. whole organs) or cells taken from the organism.

Where a nucleic acid sequence is disclosed herein, the reverse complement thereof is also expressly contemplated.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press Aspects and embodiments of the present disclosure will now be discussed with reference to the accompanying figures. Further aspects and embodiments will be apparent to those skilled in the art. All documents mentioned in this text are incorporated herein by reference in their entirety. While the present disclosure has been described in conjunction with the exemplary embodiments described below, many equivalent modifications and variations will be apparent to those skilled in the art when given this disclosure. Accordingly, the exemplary embodiments of the present disclosure set forth above are considered to be illustrative and not limiting. Various changes to the described embodiments may be made without departing from the spirit and scope of the present disclosure.

### BRIEF DESCRIPTION OF THE FIGURES

Embodiments and experiments illustrating the principles of the present disclosure will now be discussed with reference to the accompanying figures.

FIG. 1. Graph and table showing the effects of treatment from 55 weeks of age with anti-IL-11 antagonist antibody X203 or isotype-matched control IgG, on frailty of mice aged 110 weeks.

FIG. 2. Graph and table showing the effects of treatment from 55 weeks of age with anti-IL-11 antagonist antibody X203 or isotype-matched control IgG, on body temperature of mice aged 110 weeks.

FIGS. 3A and 3B. Graphs and tables showing the effects of treatment from 55 weeks of age with anti-IL-11 antagonist antibody X203 or isotype-matched control IgG, on (3A) change in fat mass and (3B) change in lean mass.

FIGS. 4A and 4B. Graphs and tables showing the effects of treatment from 55 weeks of age with anti-IL-11 antagonist antibody X203 or isotype-matched control IgG, on (4A) mass of soleus as a proportion of body weight and (4B) mass of gastrocnemius as a proportion of body weight.

FIG. 5. Image showing the level of IL-11 protein in livers obtained from 3 month old mice and 28 month old mice, as determined by western blot.

FIGS. 6A to 6D. Graphs showing the effects of treatment with anti-IL-11Rα antagonist antibody X209 or isotype-matched control IgG on the level of (6A) P21/Cdkn1a, (6B) Il1b, (6C) Il11 and (6D) Tgfb mRNA at day 7, of AML12 cells induced to undergo a cellular senescence programme.

FIGS. 7A to 7D. Graphs showing the effects of treatment with anti-IL-11 antagonist antibody X203 or isotype-matched control IgG on the level of (7A) P21/Cdkn1a, (7B) Il1b, (7C) Il11 and (7D) Tgfb mRNA at day 7, of AML12 cells induced to undergo a cellular senescence programme.

FIG. 8. Image showing the level of IL-11 protein in livers, ventricles (heart), kidneys, solei, and gastrocnemii of 110 week-old or 12 week-old male and female mice, as determined by western blot.

FIG. 9. Graph and tables showing the effects of treatment from 55 weeks of age with anti-IL-11 antagonist antibody X203 or isotype-matched control IgG, on blood urea nitrogen (BUN) levels. Dotted lines indicate the mean BUN levels of 12 week-old male (51.3 mg/dl) and female (31.8 mg/dl) C57Bl/6J mice.

FIG. 10. Graph and tables showing the effects of treatment from 55 weeks of age with anti-IL-11 antagonist antibody X203 or isotype-matched control IgG, on serum creatinine levels. Dotted lines indicate the mean serum creatinine levels of 12 week-old male (0.92 mg/dl) and female (0.77 mg/dl) C57Bl/6J mice.

FIG. 11. Graph and tables showing the effects of treatment from 55 weeks of age with anti-IL-11 antagonist antibody X203 or isotype-matched control IgG, on serum triglyceride levels. Dotted lines indicate the mean serum triglyceride levels of 12 week-old male (54 mg/dl) and female (37 mg/dl) C57Bl/6J mice.

FIG. 12. Graph and tables showing the effects of treatment from 55 weeks of age with anti-IL-11 antagonist antibody X203 or isotype-matched control IgG, on liver triglyceride levels. Dotted lines indicate the mean liver triglyceride levels of 12 week-old male (165 mg/g) and female (130 mg/g) C57Bl/6J mice.

FIG. 13. Graph and tables showing the effects of treatment from 55 weeks of age with anti-IL-11 antagonist antibody X203 or isotype-matched control IgG, on serum cholesterol levels. Dotted lines indicate the mean serum cholesterol levels of 12 week-old male (149 mg/dl) and female (90 mg/dl) C57Bl/6J mice.

FIG. 14. Graph and tables showing the effects of treatment from 55 weeks of age with anti-IL-11 antagonist antibody X203 or isotype-matched control IgG, on serum p-hydroxybutyrate levels. Dotted lines indicate the mean serum p-hydroxybutyrate levels of 12 week-old male (0.159 mM) and female (0.236 mM) C57Bl/6J mice.

FIG. 15. Western blot image showing the levels of p-LKB1, LKB1, p-mTOR, mTOR in primary human cardiac fibroblasts that are stimulated with IL11 (10 ng/ml), for 15 minutes, 2 hours, 4 hours, 6 hours, or 24 hours.

FIG. 16. IL-11 stimulates ERK-mediated phosphorylation of LKB1 serine 325(S325) and P90RSK-mediated phosphorylation of LKB1 serine 428 (S428) with subsequent inactivation of AMPK and activation of mTOR, P70RSK and S6RP. (16A) A549 cells were infected with AAV8 encoding human LKB1 (multiplicity of infection=20) for 24 hours and then stimulated with IL11 (10 ng/ml) for the indicated times (15 minutes, 2 hours, 4 hours, 6 hours, or 24 hours). Cell lysates were immunoblotted for the proteins shown. (16B) Schematic of activation of ERK (directly) and P90RSK (indirectly) by IL-11-mediated signalling and downstream effects on hallmarks of ageing. P or p; phosphorylated amino acid.

FIG. 17. Human hepatic stellate cells (17A) and hepatocytes (17B) were infected with AAV8 encoding human LKB1 (multiplicity of infection=20) for 24 hours and then stimulated with IL11 (10 ng/ml) for 2 hours. Cell lysates were immunoblotted for the proteins shown.

FIG. 18. Western blot image showing the levels of p-LKB1, LKB1, p-AMPK, AMPK in primary human cardiac fibroblasts that are unstimulated (BL+DMSO) or stimulated with IL11 for 24 hours in the presence of either DMSO, Rapamycin, Wortmannin, or U0126. IL11(10 ng/ml), Rapamycin (10 nM), Wortmannin (1 μM), U0126 (10 μM).

FIG. 19. Western blot image showing the levels of p-ERK, ERK, p-P90RSK, P90RSK, p-LKB1, LKB1, p-AMPK, AMPK, p-mTOR, mTOR, p-p70S6K, p70S6K, p-S6RP, S6RP, SMA, and GAPDH in primary human cardiac fibroblasts that are unstimulated (BL) or stimulated for 24 hours with either IL11 or TGFβ1 in the presence of X203, X209, or IgG (11E10) isotype control. IL11/TGFβ1 (10 ng/ml), IgG/X203/X209 (2 μg/ml).

FIG. 20. Western blot image showing the levels of p-ERK, ERK, p-P90RSK, P90RSK, p-LKB1, LKB1, p-AMPK, AMPK, p-Acetyl-CoA Carboxylase (p-ACC), Acetyl-CoA Carboxylase (ACC), p-mTOR, mTOR, p-p70S6K, p70S6K, p-S6RP, S6RP in primary human hepatocytes that are unstimulated (BL) or stimulated for 24 hours with either IL11, 0.5% BSA, or palmitate (saturated fatty acid) in the presence of X203, X209, or IgG (11E10) isotype control. IL11 (10 ng/ml), IgG/X203/X209 (2 μg/ml), palmitate (0.5 mM) conjugated in free BSA (0.5% BSA solution) in the ratio of 6:1.

FIG. 21. Western blot image showing the levels of p-ERK, ERK, p-P90RSK, P90RSK, p-LKB1, LKB1, p-AMPK, AMPK, p-mTOR, mTOR, p-p70S6K, p70S6K, p-S6RP, S6RP, SMA, and GAPDH in primary human hepatic stellate cells that are unstimulated (BL+DMSO) or stimulated for 24 hours with IL11 in the presence DMSO or U0126 (10 μM).

FIG. 22. Western blot image showing the levels of p-ERK, ERK, p-P90RSK, P90RSK, p-LKB1, LKB1, p-AMPK, AMPK, p-mTOR, mTOR, p-p70S6K, p70S6K, p-S6RP, S6RP in primary human hepatocytes that are unstimulated (BL+DMSO) or stimulated for 24 hours with IL11 in the presence DMSO or U0126 (10 μM).

FIG. 23. Western blot image showing the levels of p-LKB1, LKB1, p-AMPK, AMPK in primary human hepatocytes or primary human hepatic stellate cells that are unstimulated (BL) or stimulated for 24 hours with increasing concentrations (1.25, 2.5, 5, 10 or 20 ng/ml) of IL-11 or IL-6.

FIG. 24. (24A) Schematic of senescence prevention experiment for data shown in B-F. Senescent AML12 were generated by treating the cells with a sub-lethal concentration of H₂O₂ (0.75 mM) for 1 hour, followed by 23 hours recovery per day, for 7 days as described by Tripathi et. al. (2020). IgG/X209 (2 μg/ml) was added into the cultures during the 23 hours recovery period for 10 days; cells were harvested at day 10 for RNA extraction and subsequent qPCR experiments. Relative mRNA expression of (24B) Cdkn2a (p16), (24C) Cdkn1a (p21), (24D) Il1β, (24E) Il8, and (24F) Il11.

FIG. 25. (25A) Schematic of senescence reversal experiment for data shown in B-D. Senescent AML12 were generated for 7 days as described above. IgG/X203/X209 (2 μg/ml) was added into the cultures at the end of senescence induction period for 72 hours; cells were harvested at day 10 for RNA extraction and subsequent qPCR experiments. Relative mRNA expression of (25B) Cdkn1a (p21), (25C) Il1β, and (25D) Il11.

FIG. 26. IL-11 expression in cells in the kidneys of old mice. Images showing EGFP expression (grey; right column) throughout the kidneys of 110 week-old IL11:EGFP reporter mouse: in the cortex, medulla, papilla and in renal vessels. No EGFP staining was seen in age-matched control kidneys from wild-type mice (left column). Scale represents 50 μm.

FIG. 27. IL-11 expression in cells in the cardiac ventricles and atria of old mice. EGFP expression (grey) is seen in the old (110 week) IL11:EGFP reporter mouse hearts: notably in the interstitium and around blood vessels. In the atrium, specific staining is seen in the epicardium of old IL11:EGFP mice. No staining for EGFP was seen in age-matched controls or young (10 week) IL11:EGFP reporter mice. Scale represents 50 μm.

FIG. 28. IL-11 expression in cells in the lungs of old mice. EGFP expression (grey) is seen in the old (110 week) IL11:EGFP reporter mouse lung: notably peri-bronchiolar. No staining for EGFP was seen in age-matched controls or young (10 week) IL11:EGFP reporter mice. Scale represents 50 μm.

FIG. 29. IL-11 expression in cells in the spleen of old mice. EGFP expression (grey) is seen in the old (110 week) IL11:EGFP reporter mouse spleen cells and to a much lesser extent also in young (10 week) IL11:EGFP spleens. No staining for EGFP was seen in age-matched control spleen from wild-type mice.

FIG. 30. Western blot image showing the levels of IL-11 in abdominal fat tissue of wild-type male and female mice at 12 and 110 weeks of age.

FIG. 31. Livers of old Il11ra1-deleted mice have less ERK/LKB1/mTORC1 activity, more AMPK activity, and less expression of key senescence markers (p16 and p21) as compared to wild-type mice. Image showing the levels of p-ERK, ERK, p-LKB, LKB, p-AMPK, AMPK, p-mTOR, mTOR, p-p70S6K, p70S6K, p-S6RP, S6RP, p16, p21, and GAPDH in livers obtained from 10 or 110 week-old male Il11ra1+/+ (wild-type, Il11ra1 expressing) or Il1ra1−/− (Il11ra1 knockout) mice, as determined by western blot (n=3/group).

FIG. 32. Gastrocnemii of old Il11ra1-deleted mice have less ERK/LKB1/mTORC1 activity, more AMPK activity, and less expression of key senescence markers (p16 and p21) as compared to wild-type mice. Image showing the levels of p-ERK, ERK, p-LKB, LKB, p-AMPK, AMPK, p-mTOR, mTOR, p16, p21, and GAPDH in gastrocnemii obtained from 10 or 110 week-old male Il11ra1^{+/+} (wild-type, Il11ra1 expressing) or Il11ra1′ (Il11ra1 knockout) mice, as determined by western blot.

FIG. 33. Solei of old Il11ra1-deleted mice have less ERK/LKB1/mTORC1 activity, more AMPK activity, and less expression of key senescence markers (p16 and p21) as compared to wild-type mice. Image showing the levels of p-ERK, ERK, p-LKB, LKB, p-AMPK, AMPK, p-mTOR, mTOR, p-p70S6K, p70S6K, p-S6RP, S6RP, p16, p21, and GAPDH in soleii obtained from 10 or 110 week-old male Il11ra1^{+/+} (wild-type, Il11ra1 expressing) or Il11ra1^{-/-} (Il11ra1 knockout) mice, as determined by western blot.

FIG. 34. Abdominal fats of old Il11ra1-deleted mice have less ERK/LKB1/mTORC1 activity, more AMPK activity, and less expression of key senescence markers (p16 and p21) as compared to wild-type mice. Image showing the levels of p-ERK, ERK, p-LKB, LKB, p-AMPK, AMPK, p-mTOR, mTOR, p16, p21, and GAPDH in abdominal fat obtained from 10 or 110 week-old male Il11ra1^{+/+} (wild-type, Il11ra1 expressing) or Il11ra1^{-/-} (Il11ra1 knockout) mice, as determined by western blot.

FIG. 35. Old Il11ra1 KO mice had less body weight as compared to wild-type controls. Graphs and tables showing body weight for 110 week-old male and female Il11ra1^{+/+} (wild-type, Il11ra1 expressing) or Il11ra1^{+/+} (Il11ra1 knockout) mice; 2-way ANOVA.

FIG. 36. Old Il11ra1 KO mice had less body fat percentage as compared to wild-type controls. Graphs and tables showing fat percentage (fat mass (g)/body weigh (g) in %) for 110 week-old male and female Il11ra1^{+/+} (wild-type, Il11ra1 expressing) or Il11ra1^{-/-} (Il11ra1 knockout) mice as determined by echo MRI; 2-way ANOVA.

FIG. 37. Old Il11ra1 KO mice had more lean muscle mass percentage as compared to wild-type controls. Graphs and tables showing lean muscle mass percentage (muscle mass (g)/body weight (g) in %) for 110 week-old male and female Il11ra1^{+/+} (wild-type, Il11ra1 expressing) or Il11ra1^{+/+} (Il11ra1 knockout) mice as determined by echo MRI; 2-way ANOVA.

FIG. 38. Old Il11ra1 KO mice exhibited reduced frailty as compared to wild-type controls. Graphs and tables showing frailty score, which was performed blinded, for 110 week-old male and female Il11ra1^{+/+} (wild-type, Il11ra1 expressing) or Il11ra1^{+/+} (Il11ra1 knockout) mice using the protocol published by Rizzo et. al. (2018), Healthspan and lifespan measures in aging mice: Optimization of testing protocols, replicability, and rater reliability, Current Protocols in Mouse Biology, 8, e45. doi:10.1002/cpmo.45; 2-way ANOVA.

FIG. 39. Old Il11ra1 KO mice exhibited reduced body temperature as compared to wild-type controls. Graphs and tables showing body temperature for 110 week-old male and female Il11ra1^{+/+} (wild-type, Il11ra1 expressing) or Il11ra1^{-/-} (Il11ra1 knockout) mice; 2-way ANOVA.

FIG. 40. Old Il11ra1 KO mice had less abdominal fat and more muscle (soleus and gastrocnemius) as compared to wild-type controls. Graphs and tables showing the mass of (40A) abdominal fat, (40B) soleus, and (40C) gastrocnemius as a proportion of body weight for 110 week-old male and female Il11ra1^{+/+} (wild-type, Il11ra1 expressing) or Il11ra1^{-/-} (Il11ra1 knockout) mice; 2-way ANOVA.

FIG. 41. Old Il11ra1 KO mice had less fibrosis across organs as compared to wild-type controls. Graphs and tables showing collagen contents in (38A) abdominal fat, (38B) gastrocnemius, (38C) soleus and (38D) liver for 110 week-old male and female Il11ra1^{+/+} (wild-type, Il11ra1 expressing) or Il11ra1^{-/-} (Il11ra1 knockout) mice as determined by hydroxyproline assay; 2-way ANOVA.

FIG. 42. Old Il11ra1 KO mice had less fibrosis across organs as compared to wild-type controls. Graphs and tables showing collagen contents (42A) heart atria, (42B) heart ventricle, (42C) kidney, and (42D) lung for 110 week-old male and female Il11ra1^{+/+} (wild-type, Il11ra1 expressing) or Il11ra1^{-/-} (Il11ra1 knockout) mice as determined by hydroxyproline assay; 2-way ANOVA.

FIG. 43. Images showing the levels of p-ERK, ERK, p-LKB, LKB, p-AMPK, AMPK, p-mTOR, mTOR, p-p70S6K, p70S6K, p-S6RP, S6RP, p16, p21, and GAPDH in livers obtained from 12 week-old control male mice (n=3), 110 week-old male mice receiving either IgG (n=5) and or X203 (n=5) as determined by western blot.

FIG. 44. Semi-quantitative densitometry analysis of the western blot images of FIG. 43 (one-way ANOVA with Tukey's correction).

FIG. 45. Images showing the levels of p-ERK, ERK, p-LKB, LKB, p-AMPK, AMPK, p-mTOR, mTOR, p16, p21, and GAPDH in gastrocnemii obtained from 12 week-old control male mice (n=3), 110 week-old male mice receiving either IgG (n=5) and or X203 (n=5) as determined by western blot.

FIG. 46. Semi-quantitative densitometry analysis of the western blot images of FIG. 45 (one-way ANOVA with Tukey's correction).

FIG. 47. Images showing the levels of p-ERK, ERK, p-LKB, LKB, p-AMPK, AMPK, p-mTOR, mTOR, p-p70S6K, p70S6K, p-S6RP, S6RP, p16, p21, and GAPDH in solei obtained from 12 week-old control male mice (n=3), 110 week-old male mice receiving either IgG (n=5) and or X203 (n=5) as determined by western blot.

FIG. 48. Semi-quantitative densitometry analysis of the western blot images of FIG. 47 (one-way ANOVA with Tukey's correction).

FIG. 49. Images showing the levels of p-ERK, ERK, p-LKB, LKB, p-AMPK, AMPK, p-mTOR, mTOR, p16, p21, and GAPDH in abdominal fats obtained from 12 week-old control male mice (n=3), 110 week-old male mice receiving either IgG (n=5) and or X203 (n=5) as determined by western blot.

FIG. 50. Semi-quantitative densitometry analysis of the western blot images of FIG. 49 (one-way ANOVA with Tukey's correction).

FIG. 51. A neutralizing IL-11 antibody reduces the levels of IL-6 protein in the serum of old mice. Graphs showing the effects of treatment with anti-IL11 (X203) or IgG isotype control on serum IL-6 levels levels of old (110 week-old) male and female mice. Dotted lines indicate the mean serum IL-6 levels of 12 week-old control male (0.159 μg/ml) and female (0.236 μg/ml) mice. Data were analyzed by 2-way ANOVA.

FIG. 52. A neutralizing IL-11 antibody reduces multiple markers of inflammation in kidneys of old mice. Wild-type C57BL6/J mice were treated with an intra-peritoneal injection of anti-IL11 (X203; 40 mg/kg, once every three weeks) or an isotype IgG control (11E10, 40 mg/kg, once every three weeks) from 55 weeks of age until 110 weeks of age. 110 week-old mice were sacrificed and organs harvested, snap frozen and RNA extracted using standard procedures. Samples were analyzed by quantitative RT-PCR (QPCR) using primers specific for Ccl2, Ccl5, Tnfα, Il1β, Il11, Il6 or Gapdh.

FIG. 53. A neutralizing IL-11 antibody reduces multiple markers of inflammation in livers of old mice. Wild-type C57BL6/J mice were treated with an intra-peritoneal injection of anti-IL11 (X203; 40 mg/kg, once every three weeks) or an isotype IgG control (11E10, 40 mg/kg, once every three weeks) from 55 weeks of age until 110 weeks of age. 110 week-old mice were sacrificed and organs harvested, snap frozen and RNA extracted using standard procedures. Samples were analyzed by quantitative RT-PCR (QPCR) using primers specific for Ccl2, Ccl5, Tnfα, Il1β, Il11, Il6 or Gapdh.

FIG. 54. A neutralizing IL-11 antibody reduces multiple markers of inflammation in skeletal muscle (soleus) of old mice. Wild-type C57BL6/J mice were treated with an intra-peritoneal injection of anti-IL11 (X203; 40 mg/kg, once every three weeks) or an isotype IgG control (11E10, 40 mg/kg, once every three weeks) from 55 weeks of age until 110 weeks of age. 110 week-old mice were sacrificed, and organs harvested, snap frozen and RNA extracted using standard procedures. Samples were analyzed by quantitative RT-PCR (QPCR) using primers specific for Ccl2, Ccl5, Tnfα, Il1β, Il11, Il6 or Gapdh.

### EXAMPLES

In the following Examples, the inventors demonstrate that antagonism of IL-11-mediated signalling is useful for the treatment and prevention of age-related disease including frailty and age-related changes in body composition.

### Example 1: Analysis of the Ability of Antagonists of IL-11-Mediated Signalling to Treat Frailty and Age-Related Changes in Body Composition

The inventors sought to investigate whether inhibiting IL-11-mediated signalling using neutralising antibodies could reduce frailty and age-related changes in body composition, in aged mice.

C57Bl/6J mice aged 46 weeks were obtained from the Jackson Laboratory, and randomised on a 1:1 basis to receive either X203 or an isotype control IgG antibody.

X203 is a mouse anti-mouse IL-11 IgG, and is described e.g. in Ng et al., Sci Transl Med. (2019) 11(511) pii: eaaw1237 (also published as Ng, et al., “IL-11 is a therapeutic target in idiopathic pulmonary fibrosis.” bioRxiv 336537; doi: https://doi.org/10.1101/336537). X203 is also referred to as “Enx203”. X203 comprises the VH region according to SEQ ID NO:92 of WO 2019/238882 A1 (SEQ ID NO:22 of the present disclosure), and the VL region according to SEQ ID NO:94 of WO 2019/238882 A1 (SEQ ID NO:23 of the present disclosure).

From age 55 weeks (human equivalent of ˜45 years old (i.e. middle age)) to age 110 weeks (human equivalent of ˜75 years old), mice were administered with 40 mg/kg of either X203 or isotype-matched control IgG via intra-peritoneal injection every 3 weeks.

At age 110 weeks, frailty was assessed using the Mouse Frailty Index described in Rizzo et al. Curr Protoc Mouse Biol. (2018) 8(2):e45, which is hereby incorporated by reference in its entirety. In this protocol, mice are individually observed and evaluated for the absence or presence and severity of 27 different characteristics each scored as 0, 0.5, or 1 based on level of severity. A frailty index score is calculated as the cumulative score of all measures. As a separate measurement of frailty, core body temperature was recorded, as core body temperature is known to decrease with increasing frailty in mice (Reynolds et al., Mechanisms of Ageing and Development (1985) 30(2):143-52). Data were analysed by 2-way ANOVA.

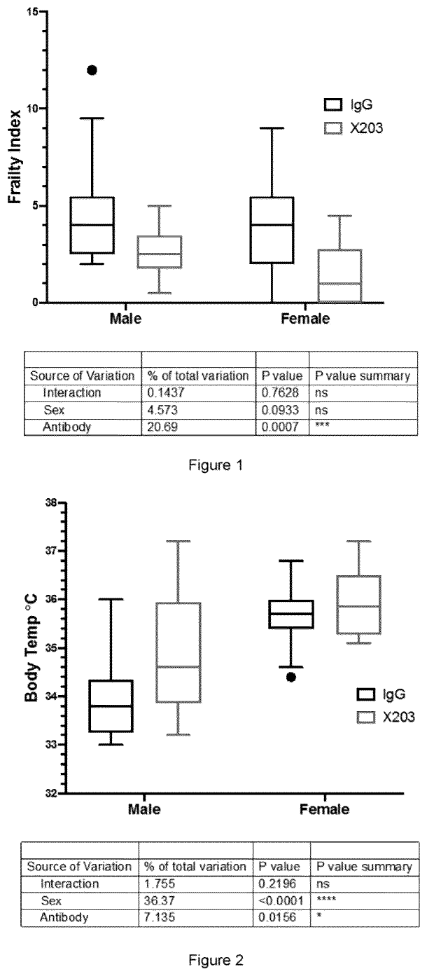

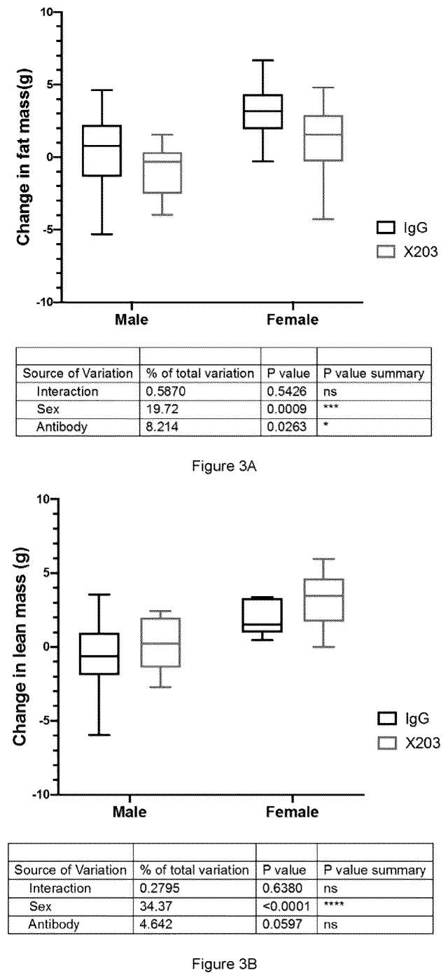

The results are shown in FIGS. 1 and 2. Treatment with X203 was found to cause a significant reduction in frailty index, and was also associated with a significantly higher body temperature.

Male mice were also found to have a lower body temperature as compared to female mice, in accordance with previous studies of frailty (e.g. Sanchez-Alavez et al. (2011) Age 33(1):89-99)).

Mice also underwent body-composition assessment via echo MRI both at baseline (age 55 weeks) and at 108-109 weeks. This enabled calculation of the change in body fat mass and lean (muscle) mass in the X203 and IgG control treatment groups. Gastrocnemius and soleus muscles were also harvested from the mice at age 110 weeks and their weights were recorded. Data were analysed by 2-way ANOVA.

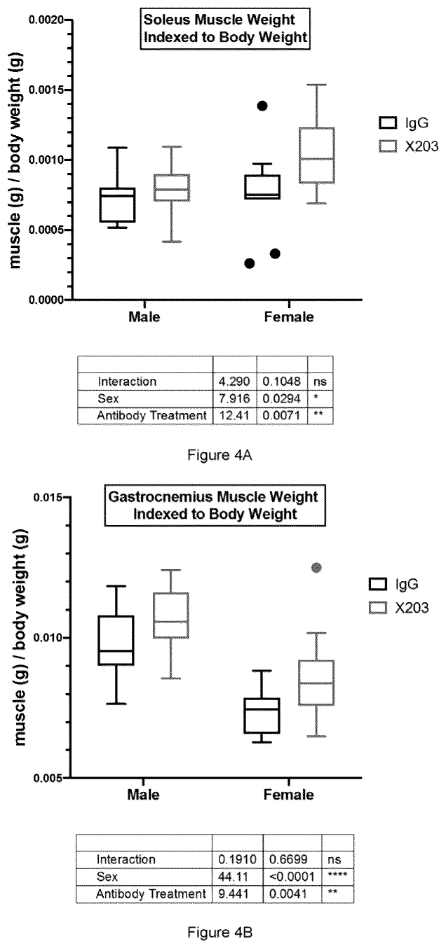

The results are shown in FIGS. 3A, 3B, 4A and 4B. Mice treated with X203 were found to have gained significantly less fat mass than mice in the IgG control treatment group (FIG. 3A). Treatment with X203 was also found to be associated with significantly greater lean mass, as compared to treatment with the IgG control antibody. In agreement with this finding, the weights of gastrocnemius and soleus muscles (as a proportion of their body weight) of mice treated with X203 were greater than those from mice treated with the IgG control antibody (FIGS. 4A and 4B).

### Example 2: Further Analysis of the Ability of Antagonists of IL-11-Mediated Signalling to Treat Age-Related Disease

### 2.1 Analysis of Behavioural Correlates of Frailty and Aging

C57Bl/6J mice are randomised to receive either X203 or IgG control antibody at a dose of 40m/kg via IP injection every 3 weeks from age 55 weeks to age 110 weeks.

At 110 weeks, mice are analysed for grip strength using a grip strength meter. Reduced grip strength is a robust marker of ageing-associated frailty in mice (see Fischer et al., Aging (2016) 8: 2370-2391, hereby incorporated by reference in its entirety).

Mice grip a wire mesh attached to the meter with both forelimbs, and are then gently pulled by the tail in a horizontal plane parallel to the base plate of the meter. Grip strength is recorded as the maximum force registered.

Mice treated with X203 display greater grip strength than mice treated with IgG control antibody.

### 2.2 Analysis of Geropathological Lesions

C57Bl/6J mice are randomised to receive either X203 or IgG control antibody at a dose of 40m/kg via IP injection every 3 weeks from age 55 weeks to age 110 weeks.

At 110 weeks, mice are euthanized, sections are prepared from heart, lung, liver, spleen, kidney and skeletal muscle tissues, subjected to hematoxylin and eosin or Masson's trichrome staining and analysed by light microscopy. Tissue sections are evaluated for ageing-associated lesions as described in Snider et al., Geroscience (2019) 40: 97-103 and Snyder et al., Geroscience (2019) 41: 455-465 (both hereby incorporated by reference in their entirety). Analysis is performed by a veterinary pathologist blinded to treatment group.

Mice treated with X203 display fewer, smaller and/or less developed geropathology lesions as compared to mice treated with IgG control antibody.

### 2.3 Analysis of Organ Fibrosis

Ageing is associated with progressive fibrosis of multiple organs, especially in the heart, lung, kidney and skeletal muscle (see e.g. Murtha et al., Aging Dis. (2019) 10:419-428, O'Sullivan et al., J. Am. Soc. Nephrol. (2017) 28: 407-420 and Etienne et al., Skelet. Muscle (2020) 10: 4, all of which are hereby incorporated by reference in their entirety).

C57Bl/6J mice are randomised to receive either X203 or IgG control antibody at a dose of 40m/kg via IP injection every 3 weeks from age 55 weeks to age 110 weeks.

At 110 weeks, global tissue collagen content is evaluated in the heart, lung, kidney and skeletal muscle tissues by hydroxyproline assay. Tissue sections from these tissues are also subjected to Masson's trichrome staining and analysed by light microscopy (blinded to treatment group).

Mice treated with X203 display less fibrosis in the tissues analysed as compared to mice treated with IgG control antibody.

### 2.4 Analysis of Markers of Cellular Senescence

Accumulation of senescent cells is a hallmark of ageing, and molecular markers of cellular senescence include senescence-associated ß galactosidase (SA-ß-gal) activity and levels of the cell cycle regulators p16^{INK4a}, p21^{CIP1} and p53 (see Wang et al., Front. Genet. (2018) 9: 247, which is hereby incorporated by reference in its entirety).

C57Bl/6J mice are randomised to receive either X203 or IgG control antibody at a dose of 40m/kg via IP injection every 3 weeks from age 55 weeks to age 110 weeks.

At 110 weeks, tissues are harvested from the mice cells of the relevant tissues and analysed for gene and/or protein expression of p16^{INK4a}, p21^{CIP1} and p53, and for SA-ß-gal activity.

Mice treated with X203 display lower levels of markers of cellular senescence as compared to mice treated with IgG control antibody.

### 2.5 Analysis of Markers of Cardiac and Renal Function

Impairment in heart and kidney function is a pathological hallmark of ageing (see e.g. Murtha et al., Aging Dis. (2019) 10:419-428, de Lucia et al. J. Gerontol. A Biol. Sci. Med. Sci. (2019) 74: 455-461 and Feridooni et al., J. Physiol. (2017) 595: 3721-3742, all of which are hereby incorporated by reference in their entirety). Aging-associated impairment of cardiac function is typically manifests as left ventricular remodelling/hypertrophy and impairment in diastolic function, and reduction in systolic function may also occur.

C57Bl/6J mice are randomised to receive either X203 or IgG control antibody at a dose of 40 mg/kg via IP injection every 3 weeks from age 55 weeks to age 110 weeks, and renal function was evaluated by analysis of serum levels of blood urea nitrogen (BUN) and creatinine using the Urea Assay Kit (ab83362, Abcam) and Creatinine Assay Kit (ab65340, Abcam) in accordance with the manufacturer's instructions.

Levels of BUN and serum creatinine was also analysed in 12 week-old male and female C57Bl/6J mice, in order to determine baseline levels. Data were analysed by 2-way ANOVA.

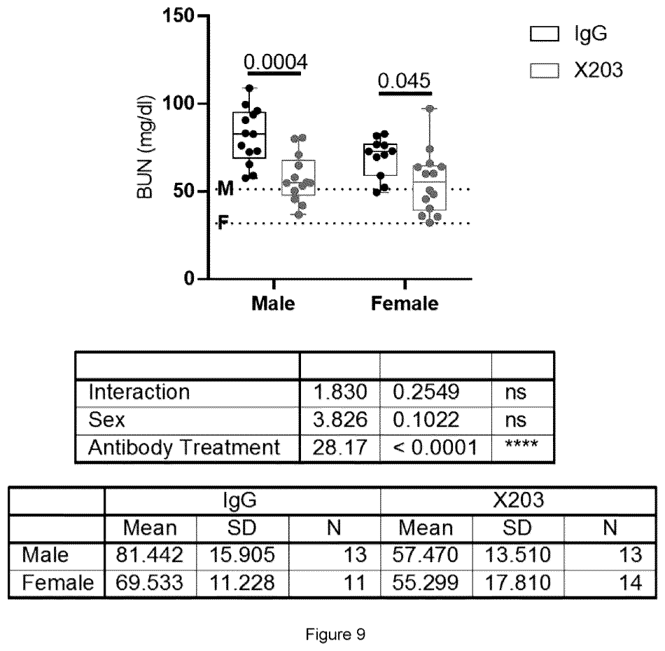

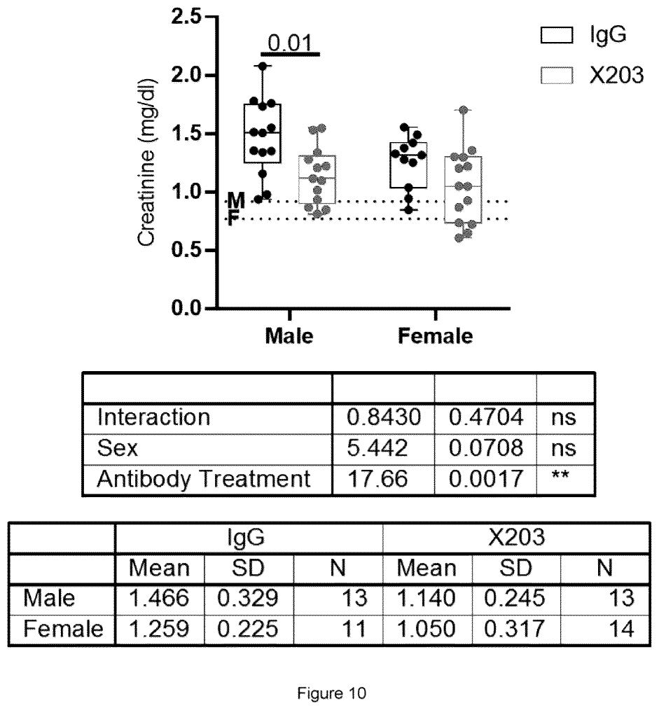

The results are shown in FIGS. 9 and 10.

FIG. 9 shows that male and female mice treated with X203 respectively displayed a 79% and 42% reduction in BUN levels relative to mice treated with IgG control antibody (using the mean BUN levels of 12 week old male (51.3 mg/dl) and female (31.8 mg/dl) mice as baseline).

FIG. 10 shows that male and female mice treated with X203 respectively displayed a 60% and 43% reduction in creatinine levels relative to mice treated with IgG control antibody (using the mean creatinine levels of 12 week old male (0.92 mg/dl) and female (0.77 mg/dl) mice as baseline).

Thus mice treated with X203 displayed improved renal function as compared to mice treated with IgG control antibody.

### 2.6 Analysis of Metabolic Function

C57Bl/6J mice were randomised to receive either X203 or IgG control antibody at a dose of 40 mg/kg via IP injection every 3 weeks from age 55 weeks to age 110 weeks, and serum triglyceride, liver triglyceride, serum cholesterol and serum p-hydroxybutyrate levels were measured using Triglyceride Assay Kit (ab65336, Abcam), Cholesterol Assay Kit (ab65390, Abcam), and Beta-Hydroxybutyrate Colorimetric Assay Kit (700190; Cayman chemicals), in accordance with the manufacturer's instructions. Levels of serum triglyceride, liver triglyceride, serum cholesterol and serum p-hydroxybutyrate were also analysed in 12 week-old male and female C57Bl/6J mice, in order to determine baseline levels. Data were analysed by 2-way ANOVA.

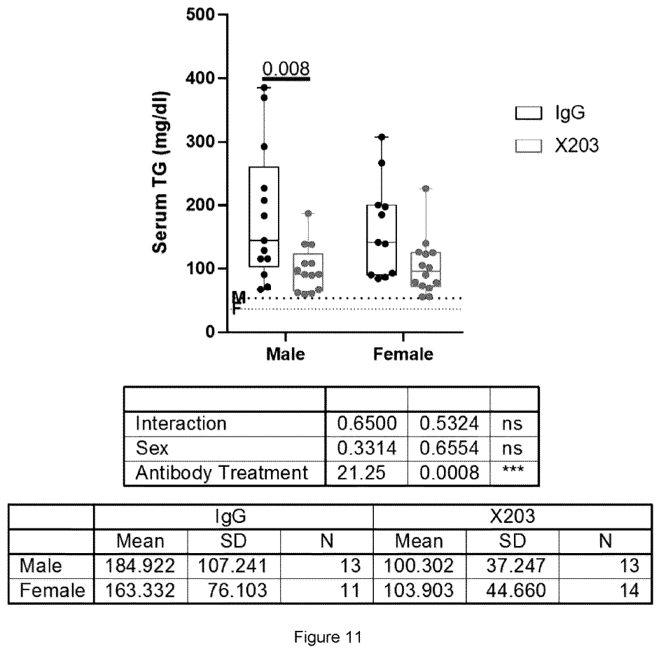

The results are shown in FIGS. 11 to 14.

FIG. 11 shows that male and female mice treated with X203 respectively displayed a 65% and 47% reduction in serum triglyceride levels relative to mice treated with IgG control antibody (using the mean serum triglyceride levels of 12 week old male (54 mg/dl) and female (37 mg/dl) mice as baseline).

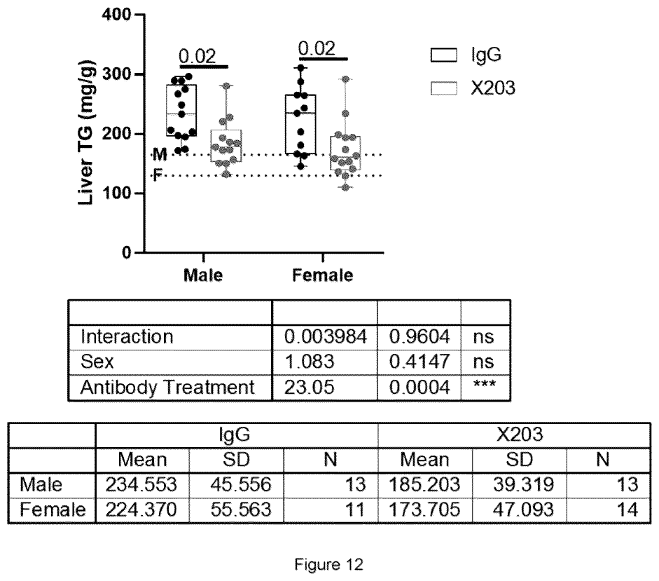

FIG. 12 shows that male and female mice treated with X203 respectively displayed a 71% and 54% reduction in liver triglyceride levels relative to mice treated with IgG control antibody (using the mean liver triglyceride levels of 12 week old male (165 mg/g) and female (130 mg/g) mice as baseline).

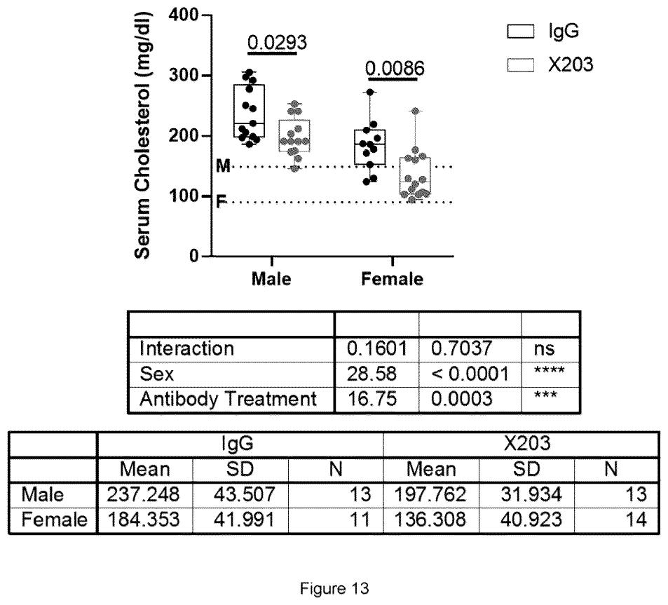

FIG. 13 shows that male and female mice treated with X203 respectively displayed a 45% and 51% reduction in serum cholesterol levels relative to mice treated with IgG control antibody (using the mean serum cholesterol levels of 12 week old male (149 mg/dl) and female (90 mg/dl) mice as baseline).

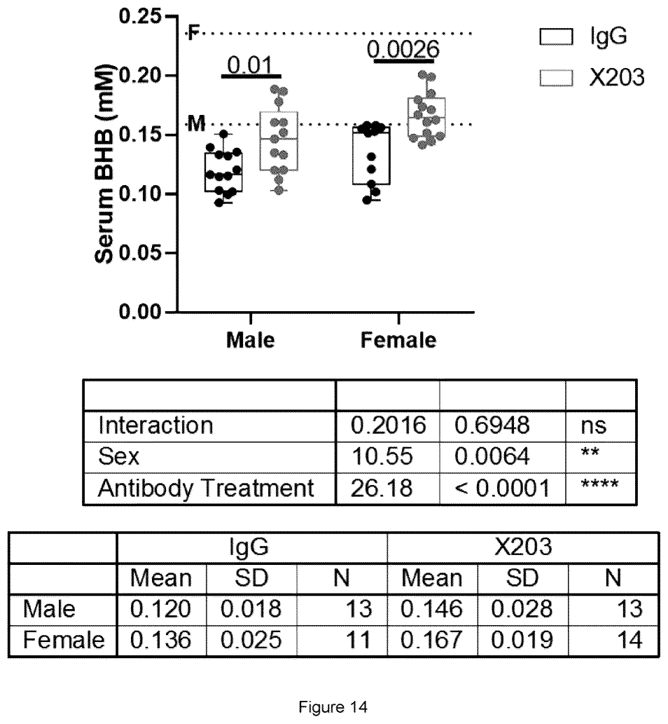

FIG. 14 shows that male and female mice treated with X203 respectively displayed a 66% and 26% reduction in serum p-hydroxybutyrate levels relative to mice treated with IgG control antibody (using the mean serum p-hydroxybutyrate levels of 12 week old male (0.159 mM) and female (0.236 mM) mice as baseline).

Thus treatment with X203 was found reduce aging-associated hypertriglyceridemia, steatosis (liver fat accumulation) and hypercholesterolemia. Treatment with X203 was found to increase levels of circulating β-hydroxybutyrate, which is a peripheral marker of fatty acid oxidation and ketone production. Taken together, these data indicate that antagonism of IL-11-mediated signalling reduces aging-associated decline in metabolic function.

### Example 3: Evaluation of IL-11-Mediated Signalling and Senescence

### 3.1 Levels of IL-11 Protein in Young and Old Mice

The inventors evaluated the expression of IL-11 protein in the livers of 28 month-old and 3 month-old C57Bl/6J mice.

Briefly, livers were harvested from mice and homogenized in radioimmunoprecipitation assay (RIPA) lysis and Extraction Buffer (ThermoFisher Scientific) containing protease and phosphatase inhibitors (Roche), followed by centrifugation to clear the lysate. Protein concentrations were determined by Bradford assay (Bio-Rad). Equal amounts of protein lysates were separated by SDS-PAGE, transferred to PVDF membranes, blocked for 1 h with 3% BSA, and incubated overnight with X203 (1:2000) or anti-GAPDH (1:1000, CST). Protein bands were visualized using the ECL detection system (Pierce) with the appropriate secondary antibodies: anti-mouse HRP and anti-rabbit HRP (1:5000, CST). Proteins were visualized using the ECL detection system (Pierce) with appropriate secondary antibodies.

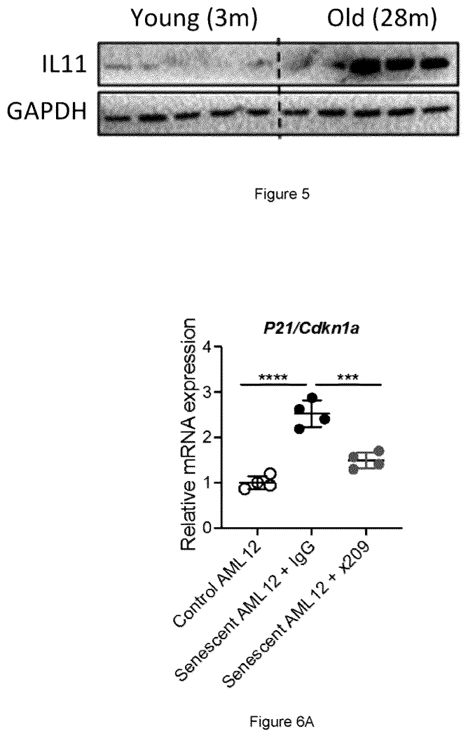

The results are shown in FIG. 5. The level of IL-11 protein was found to be greater in the livers of 28 month-old mice as compared to 3 month-old mice.

In further experiments, the inventors evaluated the expression of IL-11 protein in the livers, ventricles, kidneys, gastrocnemii, and solei of 110 week-old and 12 week-old male and female C57Bl/6J mice. Total protein extracts from livers, ventricles, kidneys, gastrocnemii, and solei in RIPA lysis and Extraction Buffer and analysed by western blot as described above.

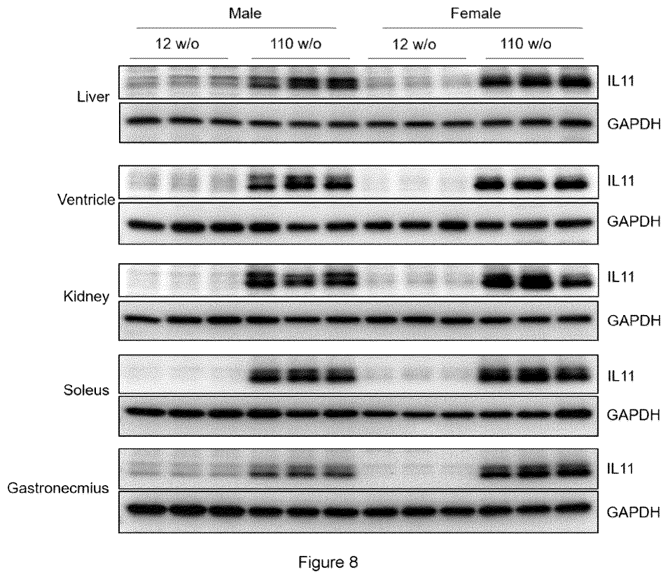

The results are shown in FIG. 8. The level of IL-11 protein was found to be greater in the livers, ventricles, kidneys, gastrocnemii, and solei of 110 week-old mice as compared to 12 week-old mice.

### 3.2 Effects of Antagonism of IL-11-Mediated Signalling on Expression of Markers of Senescence by Cells in an In Vitro Model of Senescence Induction

The inventors next investigated the effect of antagonising IL-11-mediated signalling on the expression of markers of senescence and inflammation by senescent cells in vitro, using X203 or X209.

X209 is a mouse anti-mouse IL-11Rα IgG, and is described e.g. in Widjaja et al., Gastroenterology (2019) 157(3):777-792. X209 is also referred to as “Enx209”, and comprises the VH region according to SEQ ID NO:7 of WO 2019/238884 A1 (SEQ ID NO:24 of the present disclosure), and the VL region according to SEQ ID NO:14 of WO 2019/238884 A1 (SEQ ID NO:25 of the present disclosure).

Senescent hepatic mouse cells were generated from cells of the mouse hepatocyte cell line AML12 as described in Tripathi et al., Star Protocols (2020) 1:100064 (hereby incorporated by reference in its entirety).

Briefly, AML12 cells were grown to 50% confluency and senescence was induced by treating the cells with a sub-lethal concentration of H₂O₂ (0.75 mM) for 1 h, followed by 23 h recovery per day, for a total of 7 days (as described in Tripathi et al., Star Protocols (2020) 1:100064). From day 2 onwards, 2 ug/ml of X209/X203 or isotype-matched control IgG was added to the cultures during the 23h recovery period.

Total RNA was isolated at day 7, and levels of p21 (Cdkn1a), Il11b, Il11 and Tgfb gene expression were evaluated by qRT-PCR. Briefly, total RNA was extracted from cell lysates using Trizol (Invitrogen) followed by RNeasy column (Qiagen) purification. cDNAs were synthesized with iScript™ cDNA synthesis kit (Bio-Rad) according to manufacturer's instructions. Gene expression analysis was performed with either TaqMan (Applied Biosystems) or fast SYBR green (Qiagen) using the StepOnePlus Real-Time PCT System (Applied Biosystems) over 40 cycles. Expression data for p21 (Cdkn1a), Il1b, Il11 and Tgfb were normalised to Gapdh expression, and fold change was calculated using the 2^{-ΔΔCt} method.

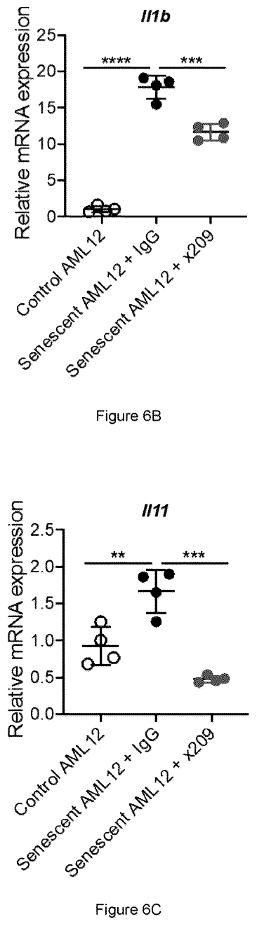

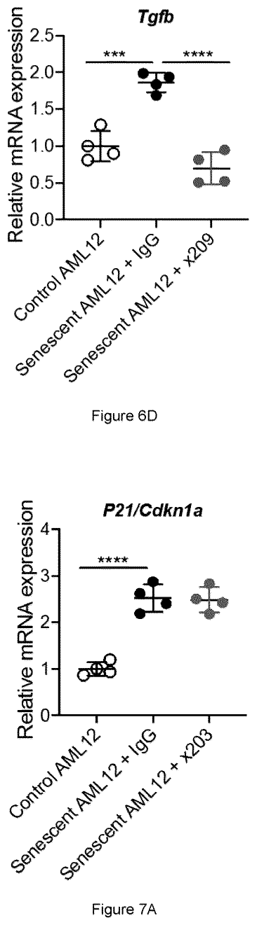

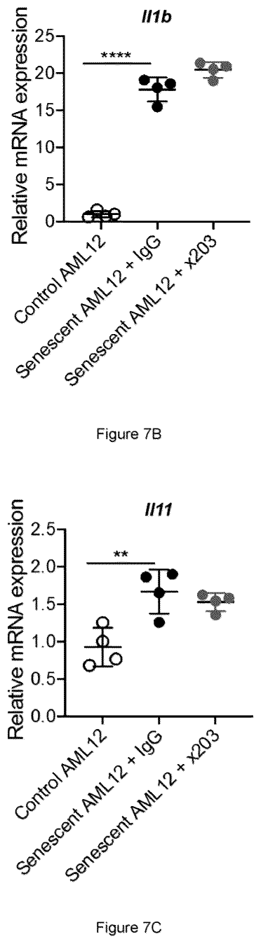

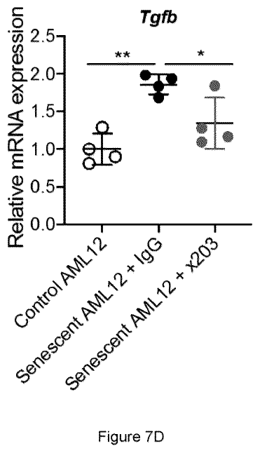

The results are shown in FIGS. 6A to 6D, and FIG. 7A to 7D.

Treatment with X209 was found to significantly reduce gene expression of p21 (Cdkn1a), Il11b, Il11 and Tgfb by AML12 cells undergoing a programme of senescence induction (FIGS. 6A to 6D).

Treatment with X203 was found to significantly reduce gene expression of Tgfb by AML12 cells undergoing a programme of senescence induction (FIG. 7D), and expression of Il11 also trended towards a reduced level (FIG. 7C).

### 3.3 Effects of Antagonism of IL-11-Mediated Signalling on Expression of Markers of Senescence by Cells in an In Vitro Model of Senescence Induction Using Human Cells

Primary human hepatocytes or AML12 cells are grown to 50% confluency and senescence is induced by treating the cells with a sub-lethal concentration of H₂O₂ (0.75 mM) for 1 h, followed by 23 h recovery per day, for a total of 7 days. From day 2 onwards, 2 ug/ml of X209/X203 or isotype-matched control IgG is added to the cultures during the 23h recovery period.

After 7 days, total RNA is isolated from the cells, and isolated in order to evaluate expression of p21 (Cdkn1a), Il1b, Il11 and Tgfb as described in Example 3.2, as well as p16 and p53. The cells are also subjected to phenotypic evaluation of cellular hypertrophy and nuclear enlargement. The cells are also analysed for chromatin condensation, DNA damage, replication capacity, ROS generation and oxidative stress, SASP gene and protein expression, energy phenotyping and mitochondrial activity/damage, cellular glycolysis, and for the activity of autophagy and nutrient sensing pathways (such as mTOR, AMPK).

Treatment with the antibody antagonists of IL-11-mediated signalling is shown to reduce the level of markers of cellular senescence as compared to treatment with IgG control antibody, indicating that antagonism of IL-11-mediated signalling is able to inhibit induction of cellular senescence.

### 3.4 Effects of Antagonism of IL-11-Mediated Signalling on Expression of Markers of Senescence by Cells in an In Vitro Model of Senescence Induction Using Human Cells

Primary human hepatocytes or AML12 cells are grown to 50% confluency and senescence is induced by treating the cells with a sub-lethal concentration of H₂O₂ (0.75 mM) for 1 h, followed by 23 h recovery per day, for a total of 7 days.

From day 7, cells are treated with 2 ug/ml of X209/X203 or isotype-matched control IgG.

At day 14, total RNA is isolated from the cells, and isolated in order to evaluate expression of p21 (Cdkn1a), Il1b, Il11 and Tgfb as described in Example 3.2, as well as p16 and p53. The cells are also subjected to phenotypic evaluation of cellular hypertrophy and nuclear enlargement. The cells are also analysed for chromatin condensation, DNA damage, replication capacity, ROS generation and oxidative stress, SASP gene and protein expression, energy phenotyping and mitochondrial activity/damage, cellular glycolysis, and for the activity of autophagy and nutrient sensing pathways (such as mTOR, AMPK).

Treatment with the antibody antagonists of IL-11-mediated signalling is shown to reduce the level of markers of cellular senescence as compared to treatment with IgG control antibody, indicating that antagonism of IL-11-mediated signalling is able to reverse induction of cellular senescence.

### Example 4: In Vitro Evaluation of the Effect of IL-11-Mediated Signalling on Pathways of Ageing

Cellular senescence is one of the nine “Hallmarks of ageing”, pathways that drive the ageing process (López-Otín et al. 2013, Cell 153 (6): 1194-1217).

The inventors investigated the effect of IL-11-mediated signalling on key hallmarks of ageing: nutrient sensing (Liu and Sabatini 2020, Nature Reviews. Molecular Cell Biology 21 (4): 183-203), loss of proteostasis (Kaushik and Cuervo 2015, Nature Medicine 21 (12): 1406-15) and cellular senescence (Dolgin 2020, Nature Biotechnology 38 (12): 1371-77). The ageing pathways associated with these Hallmarks are: insulin/IGF-1 signaling (IIS), mammalian target of rapamycin 1 (mTORC1), AMP-activated kinase (AMPK) and MEK/ERK pathway.

### 4.1 Effect of IL-11 on Ageing Pathways in Human Cardiac Fibroblasts

Primary human cardiac fibroblasts were stimulated with IL-11 (10 ng/ml), for 15 mins, 2, 4, 6, or 24 hours and the levels of phosphorylated LKB1 (p-LKB1), LKB1, phosphorylated mTOR (p-mTOR) and mTOR were measured by western blot.

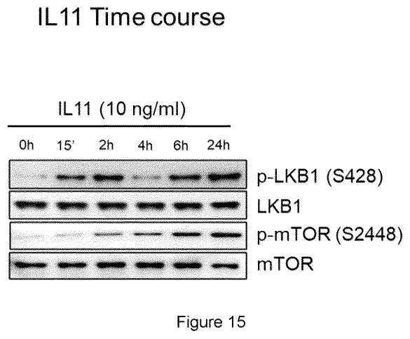

Stimulation with IL-11 increases the level of phosphorylated LKB1 and phosphorylated mTOR (FIG. 15). This data suggests that IL-11-mediated signalling plays a role in inactivating LKB1 through phosphorylation which in turns leads to activation of mTORC1. This is a novel finding because to the inventors' knowledge, LKB1 has only been thought to be constitutively active and not regulated by phosphorylation.

### 4.2 IL-11 Stimulates Phosphorylation of LKB1

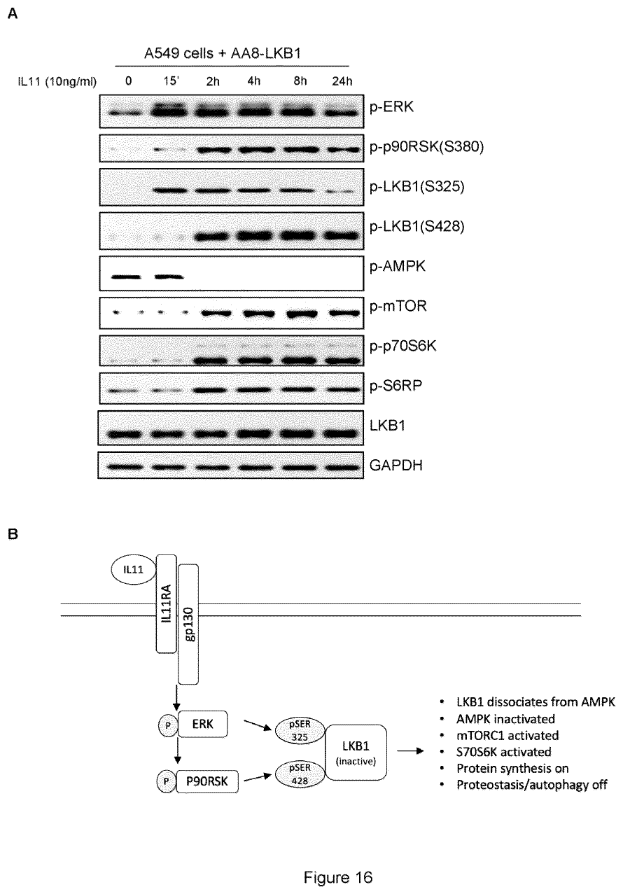

To determine the mechanism through which IL-11 stimulates phosphorylation of LKB1, A549 cells were engineered to overexpress human LKB1 (AA8-LKB1 in FIG. 16A) and treated with IL-11. Western blot analysis revealed that IL-11 stimulates ERK-mediated phosphorylation of LKB1 Serine 325 (S325), and P90RSK-mediated phosphorylation of LKB1 serine 428 (S428).

The inventors identified that IL-11-mediated signalling activates ERK and P90RSK to double phosphorylate LKB1 that causes its inactivation and drives key hallmarks of ageing. A schematic outlining the role of IL-11 is shown in FIG. 16B. The Examples disclosed herein confirm that inhibition of IL-11-mediated signalling with a neutralising antibody or deletion of Il11ra prevents ERK activation, stimulates LKB1/AMPK and inhibits mTORC1/P70S6K while diminishing markers of senescence (P16 and p21), thereby inhibition/inactivation of IL-11 co-ordinately targets multiple critical ageing processes (metabolism, inflammation, protein translation and senescence).

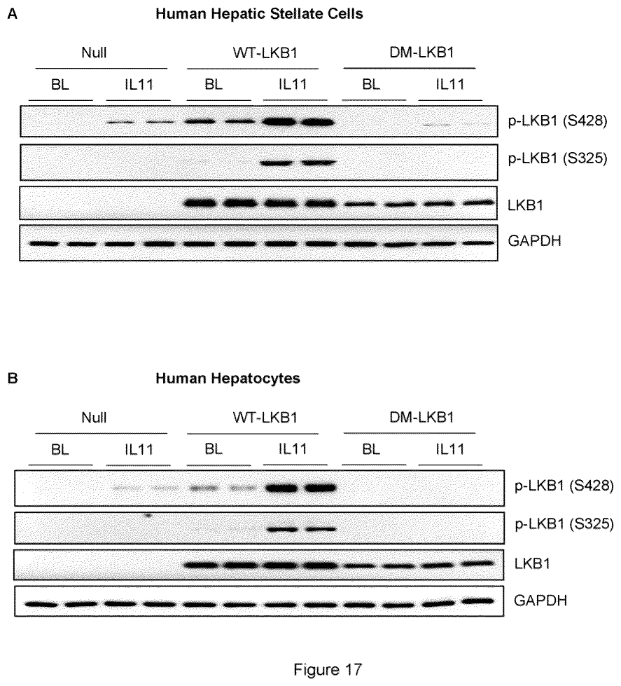

Human hepatic stellate cells (FIG. 17A) and human hepatocytes (FIG. 17B) were engineered to overexpress the wild-type LKB1 and a double mutant S325A, S428A (DM-LKB1). FIGS. 17A and 17B confirm that stimulation with IL-11 induces phosphorylation at S428 (ERK-site) and S325 (P90RSK-site) in the WT LKB1 cells. As expected, phosphorylation is not observed in the double mutant LKB1 cells.

The inventors attribute the lack of detection of S325 phosphorylation in the cells that do not overexpress LKB1 (Null) to the low sensitivity of the antibody.

### 4.3 Effect of Rapamycin, Wortmannin or U0126 on IL-11-Stimulated Human Cardiac Fibroblasts

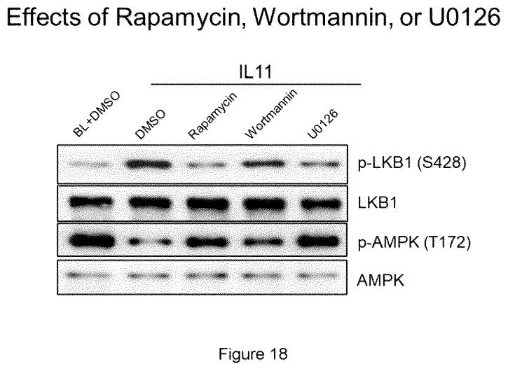

Primary human cardiac fibroblasts were stimulated with IL-11 for 24 hours in the presence of either DMSO, Rapamycin (mTOR inhibitor), Wortmannin (phosphoinositide 3-kinases (PI3K) inhibitor), or U0126 (MEK1/2 inhibitor) and the levels of p-LKB1 and p-AMPK were measured by western blot (FIG. 18).

The level of p-LKB1 decreases whereas the level of p-AMPK increases when IL-11 stimulated fibroblasts are treated with rapamycin, Wortmannin or U0126. This data shows that each of rapamycin, wortmannin or U0126 partially prevent phosphorylation of LKB1 by IL-11 and subsequent dephosphorylation of AMPK, thus confirming that IL-11 promotes MEK/ERK, AMPK and mTORC1 pathways.

### 4.4 Effect of Antagonism of IL-11-Mediated Signalling on Ageing Pathways in Human Cardiac Fibroblasts

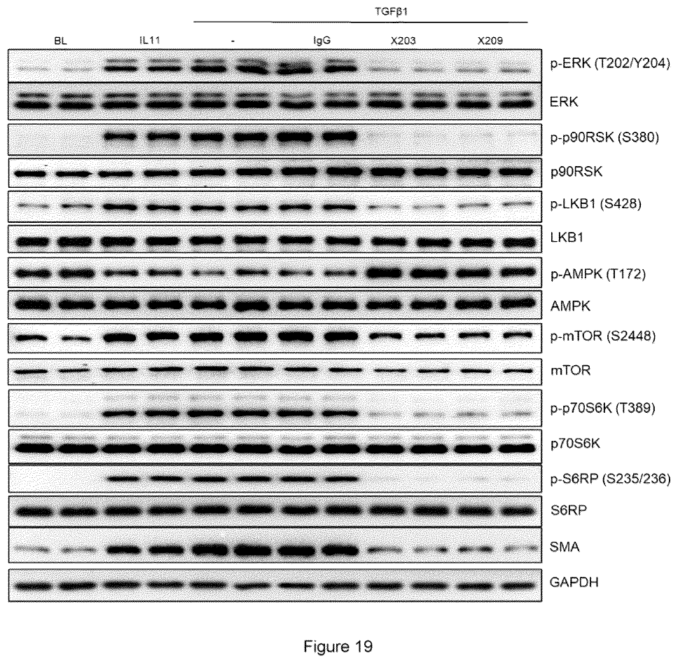

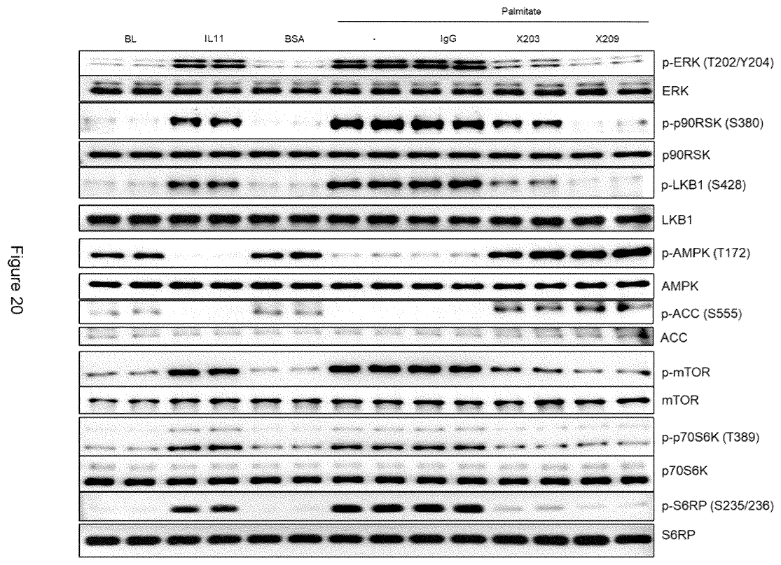

Primary human cardiac fibroblasts were stimulated with IL11 or TGFβ1, or unstimulated (BL). FIG. 19 shows the levels of various markers as determined by western blot.

Firstly, this data further confirms the role of IL-11 as an activator of ERK to phosphorylate and inactivate LKB1 directly and indirectly through p90RSK. As explained in Example 4.1, LKB1 has until now been thought to be constitutively active and not regulated by phosphorylation. IL-11-induced p-ERK and p-p90RSK act to phosphorylate and inactivate LKB1 which in turn leads to inactivation (dephosphorylation) of AMPK. Inactive AMPK can no longer activate members of the TSC complex, which act to inhibit mTORC1. As such, mTORC1 becomes activated (phosphorylated). Activated mTORC1 phosphorylates and activates P70S6K and S6-ribosomal protein (RPS6 or S6RP) to stimulate protein synthesis and many other pro-aging pathways including inhibition of autophagy that impairs proteostasis.

Both X203 (anti-IL-11 antagonist antibody) and X209 (anti-IL-11Rα antagonist antibody) significantly reduce the level of phosphorylation of markers ERK, p90RSK, LKB1, mTOR, p70S6K and S6RP. Treatment with X203 or X209 essentially reverses the effect of IL-11/TGFβ1 stimulation on these markers to the levels observed in the unstimulated control.

### 4.5 Effect of Antagonism of IL-11-Mediated Signalling on Ageing Pathways in Human Hepatocytes

Primary human hepatocytes (stimulated with IL-11, BSA or palmitate, or unstimulated) were treated with X203 (anti-IL-11 antagonist antibody) and X209 (anti-IL-11Rα antagonist antibody). FIG. 20 shows that treatment with either X203 or X209 reduces the levels of phosphorylated markers p-ERK, p-p90RSK, p-LKB1, p-mTOR, p-p70S6K and p-S6RP, while stimulating AMPK and Acetyl-CoA Carboxylase (ACC), as evidenced by an increase in the levels of p-AMPK and p-ACC. Treatment with X203 or X209 essentially reverses the effect of palmitate stimulation on these markers to the levels observed in the unstimulated control.

### 4.6 Effect of IL-11 on Ageing Pathways in Primary Human Hepatic Stellate Cells and Human Hepatocytes in the Presence of U0126

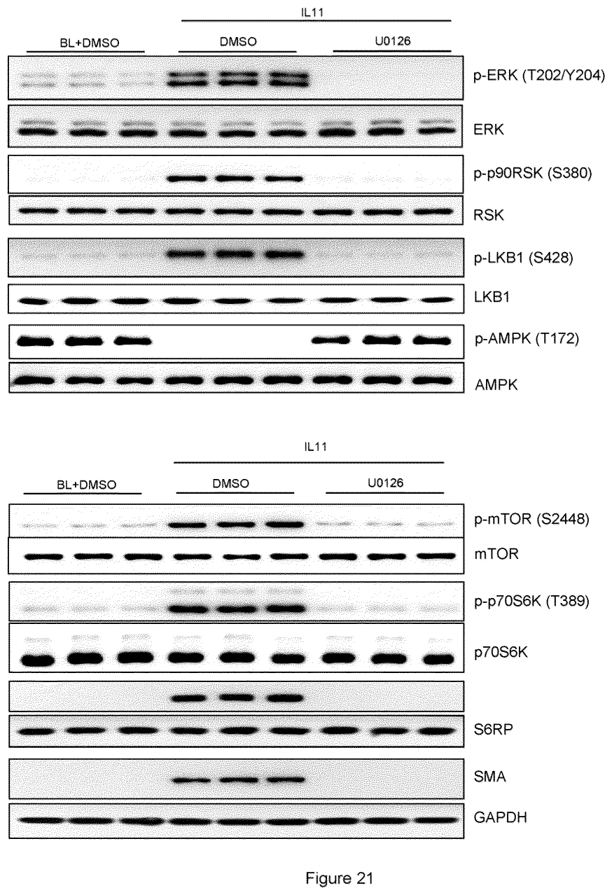

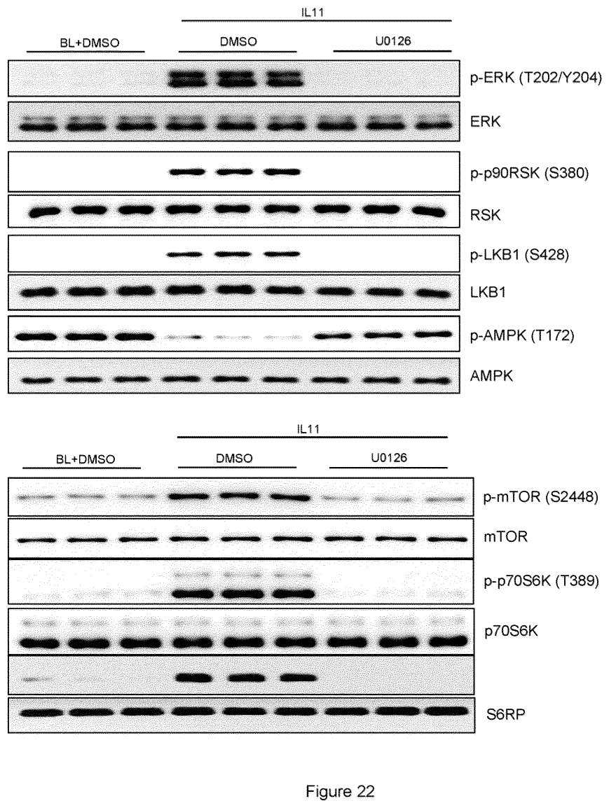

Primary human hepatic stellate cells and human hepatocytes were stimulated for 24 hours with IL-11 in the presence DMSO or U0126. As discussed in Example 4.4, the inventors observed that IL-11 treatment stimulates p-mTOR, p-70S6K and p-S6RP. In line with the inventors' hypothesis, administration of U0126 to IL-11-stimulated cells prevents ERK-mediated inactivation of LKB1/AMPK that stops IL11-induced mTORC1/P70S6K activation (FIG. 21 and FIG. 22, respectively).

### 4.7 Comparative Effect of IL-11 and IL-6 on Ageing Pathways in Primary Human Hepatic Stellate Cells and Human Hepatocytes

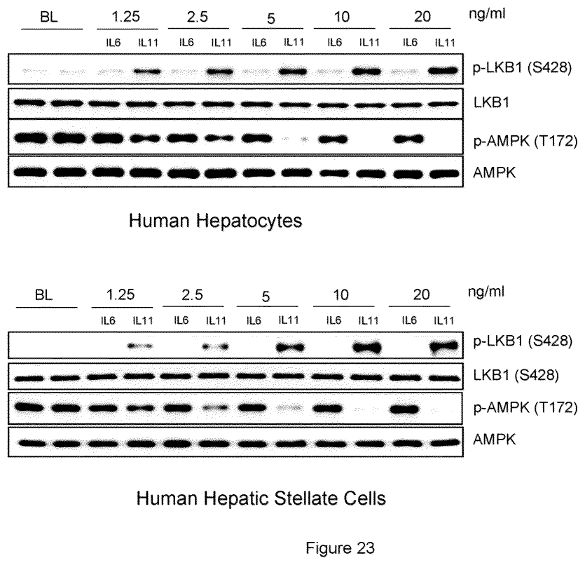

Primary human hepatic stellate cells (HSCs) and human hepatocytes were stimulated for 24 hours with increasing concentrations (1.25-20 ng/ml) of IL-11 or IL-6. Unlike IL-11, IL-6 does not induce inactivation of LKB1 or activation of AMPK even at the highest concentration (FIG. 23). The effect of IL-11 on these markers is observable at concentrations as low as 1.25 ng/ml in both HSCs and hepatocytes.

### 4.8 Effect of X209 and X203 in an In Vitro Model of Senescence

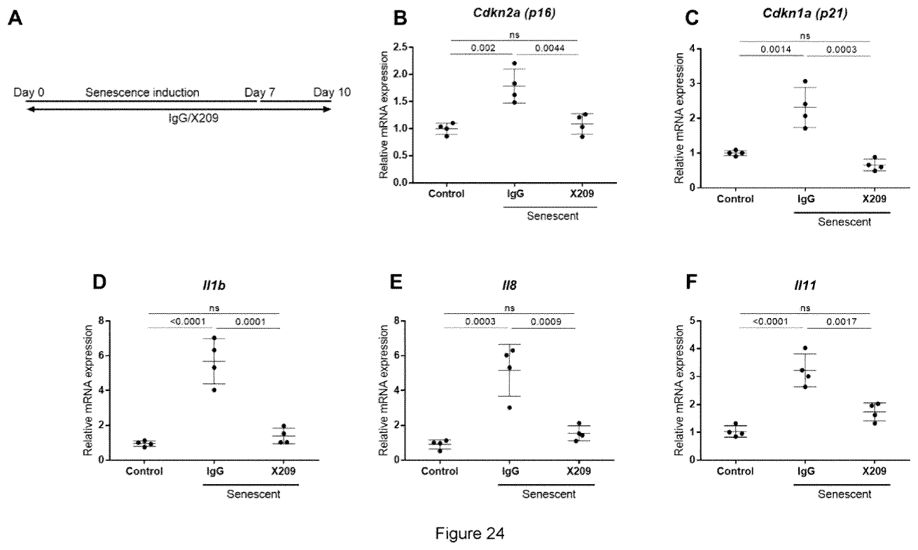

A senescence prevention experiment was carried out as shown in FIG. 24A. Briefly, senescent AML12 were generated by treating the cells with a sub-lethal concentration of H₂O₂ as described by Tripathi et. al. (2020) STAR Protocols 1 (2): 100064. IgG/X209 (2 μg/ml) was added into the cultures during the 23 hours recovery period for 10 days. Relative mRNA expression of Cdkn2a (p16), Cdkn1a (p21), Il1β, Il8, and Il11, as measured by qPCR is shown in FIGS. 24B, 24C, 24D, 24E and 24F, respectively. Markers of senescence (P16 and p21) and markers of inflammation (IL-1b, IL-11 and IL-6) are diminished in cells that have undergone IL-11-mediated signalling antagonism by X209.

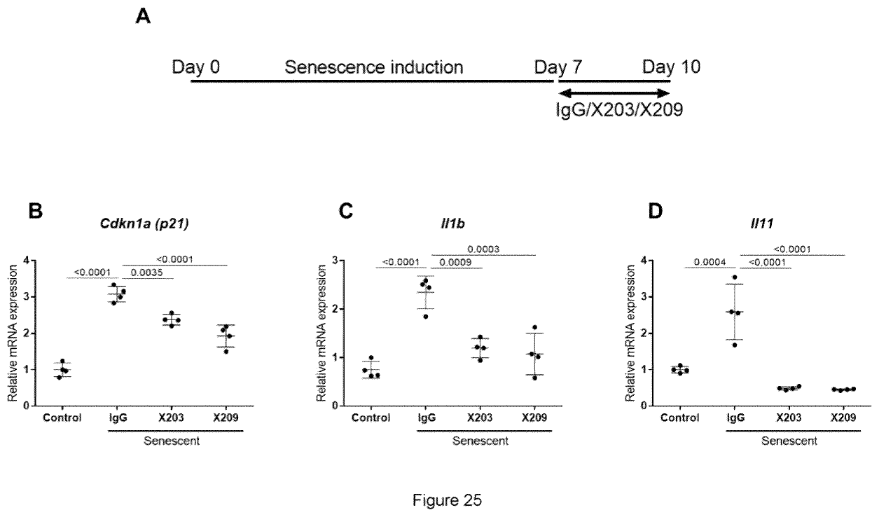

A senescence reversal experiment was carried out as shown in FIG. 25A. Briefly, senescent AML12 were generated for 7 days as described above, but IgG/X203/X209 (2 μg/ml) was added into the cultures at the end of senescence induction period for 72 hours. Both X203 and X209 antagonists of IL-11-mediated signalling significantly reduce senescence marker p21 (FIG. 25B) and inflammation markers IL-1 b (FIG. 25C) and IL-11 (FIG. 25D).

In summary, in in vitro models of senescence (AML12 cells), anti-IL-11 or anti-IL-11RA antagonists prevent and also reverse cellular senescence.

### Example 5: Detection of IL-11 Upregulation with Age In Vivo

### 5.1 IL-11 Upregulation in Tissues of Old EGFP Reporter Mouse

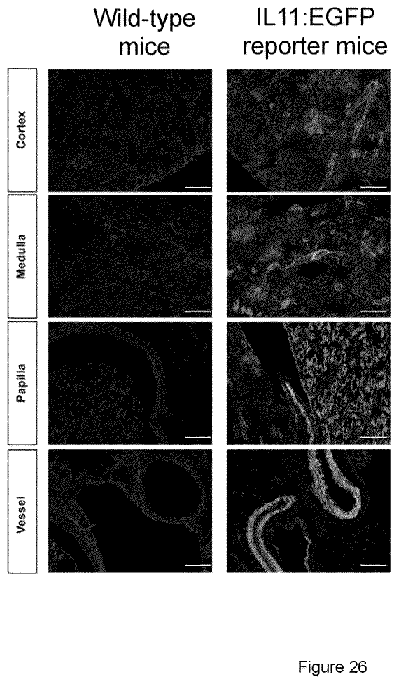

IL11:EGFP reporter mice (Il11-Egfp^{+/-}; (Widjaja et al. 2021, Science Translational Medicine 13 (597))) were generated and allowed to reach the age of 110 weeks. FIG. 26 shows immunofluorescence images of kidneys of the ‘old’ IL11:EGFP reporter mice as compared to wild type 110-week old mice. No EGFP staining was detected in age-matched control kidneys from wild-type mice.

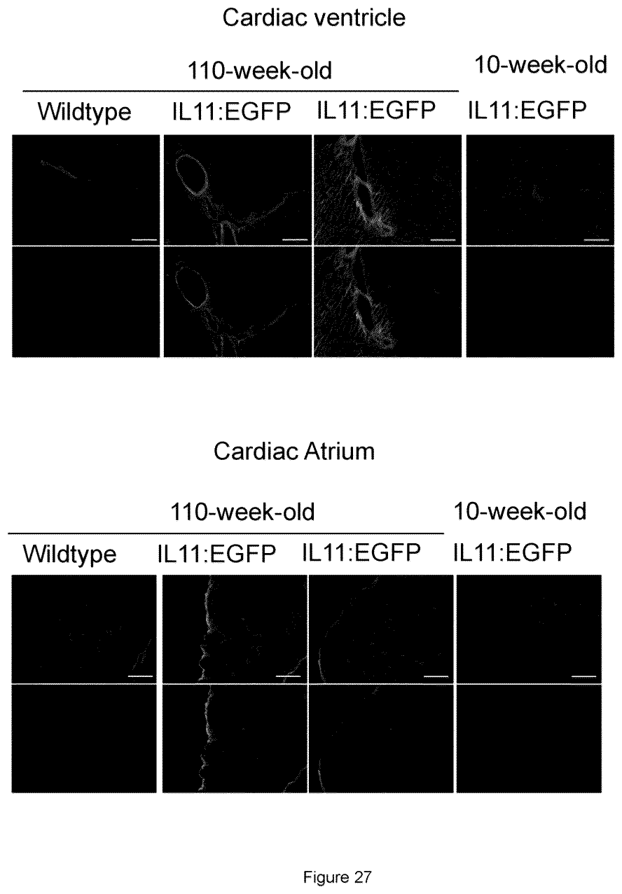

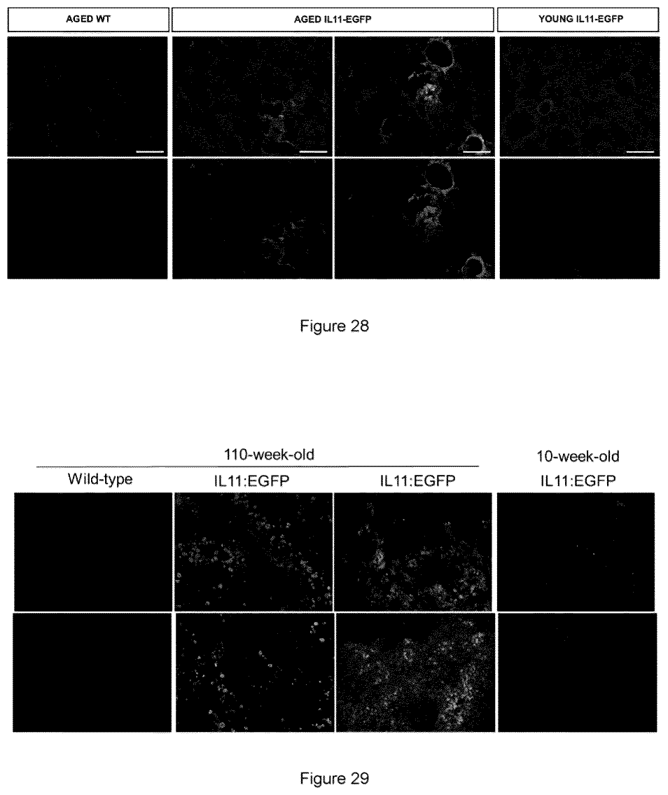

IL-11 expression in cells in the cardiac ventricles and atria (FIG. 27), lung (FIG. 28) and spleen (FIG. 29) was observed in the old (110 week) IL11:EGFP reporter mice. No (or much less) staining for EGFP was seen in age-matched controls or young (10 week) IL11:EGFP reporter mice. IL-11 is upregulated in tissue of old mice and these findings support the role of IL-11 in promoting pathways of ageing.

### 5.2 IL-11 Upregulation in Abdominal Fat of Old WT Mice

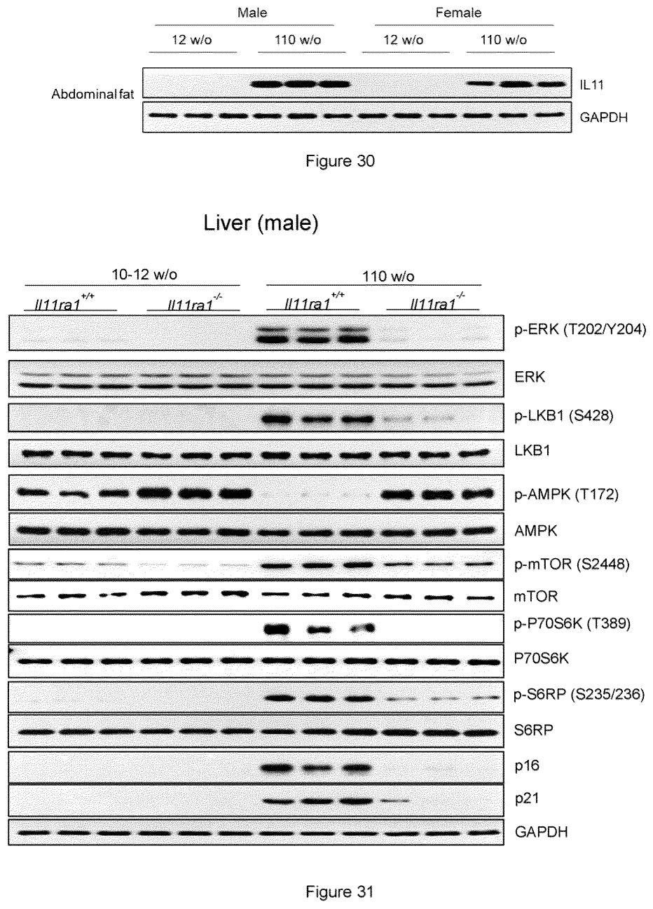

Western blot analysis (FIG. 30) demonstrates that IL-11 is upregulated in abdominal fat tissue of 110-week old wild-type mice.

### Example 6: In Vivo Studies on IL1IRA1 Knock-Out Mice

### 6.1 Effect of Il11ra1-Deletion on Ageing Pathways

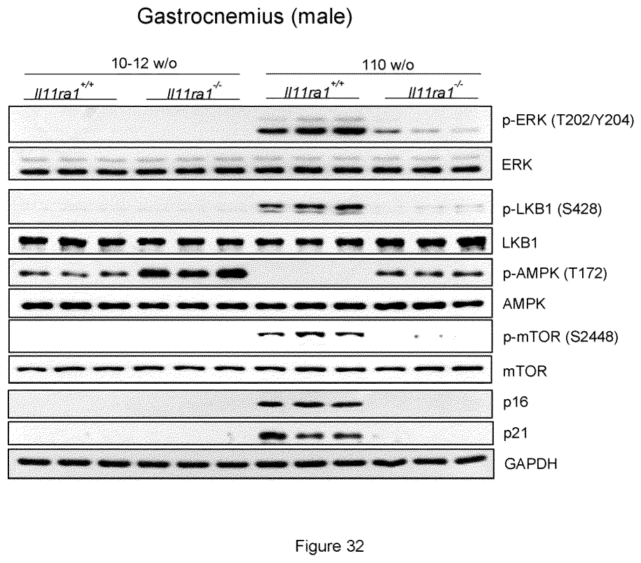

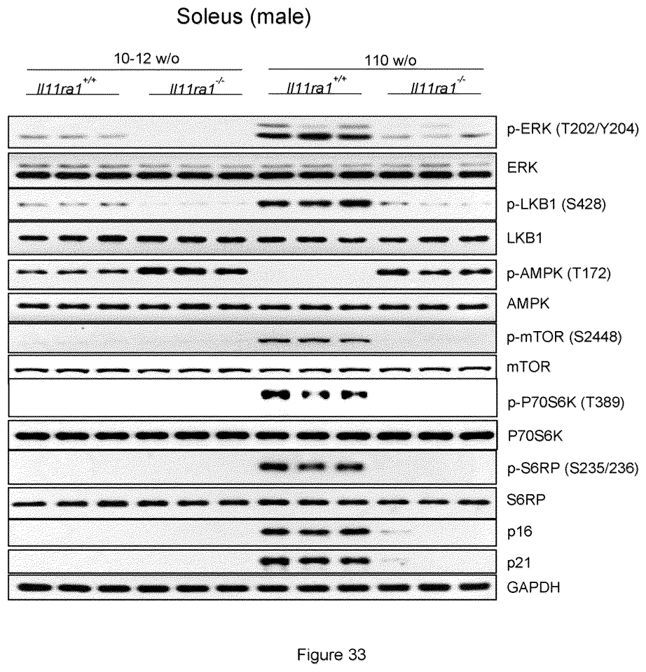

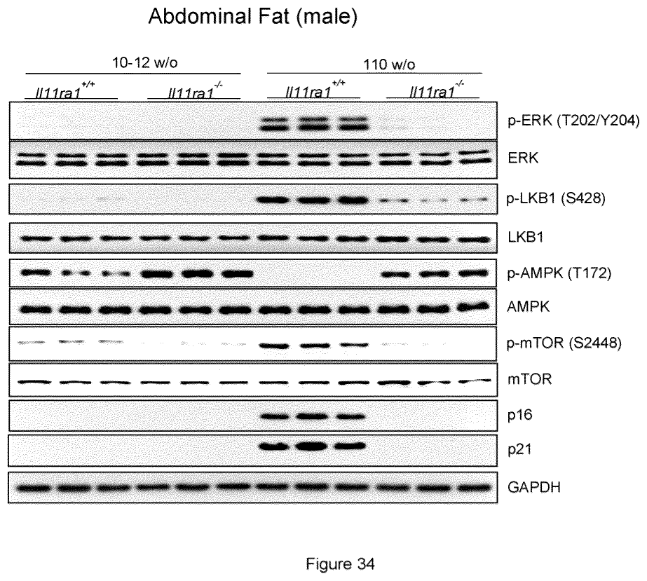

Il11ra1-KO male mice were generated and allowed to reach the age of 10-12 weeks (young) and 110 weeks (old). FIG. 31 demonstrates that livers of old Il11ra1-deleted mice have less ERK/LKB1/mTORC1 activity, more AMPK activity, and less expression of key senescence markers (p16 and p21) as compared to wild-type mice, and young Il11ra1-deleted mice, thus confirming the role of IL-11 in promoting pathways of ageing. The same results are observed in the gastrocnemii (FIG. 32), solei (FIG. 33) and abdominal fat (FIG. 34) of old Il11ra1-deleted mice.

### 6.1 Effect of Il11ra1-Deletion on the Phenotype

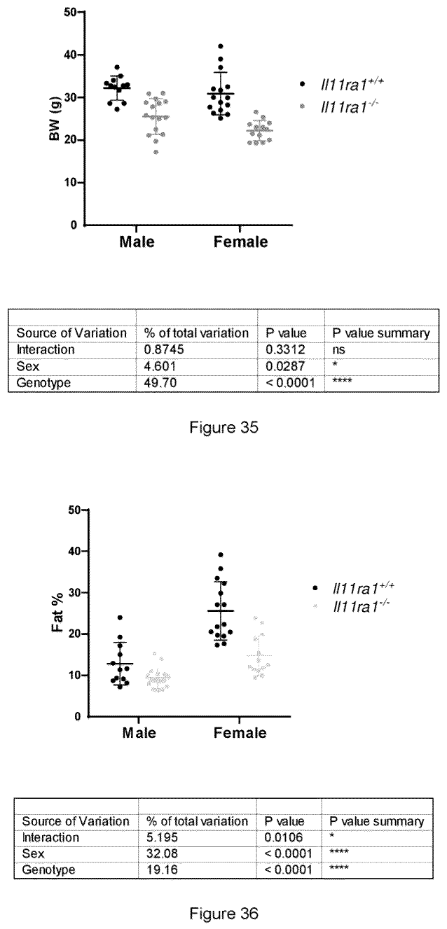

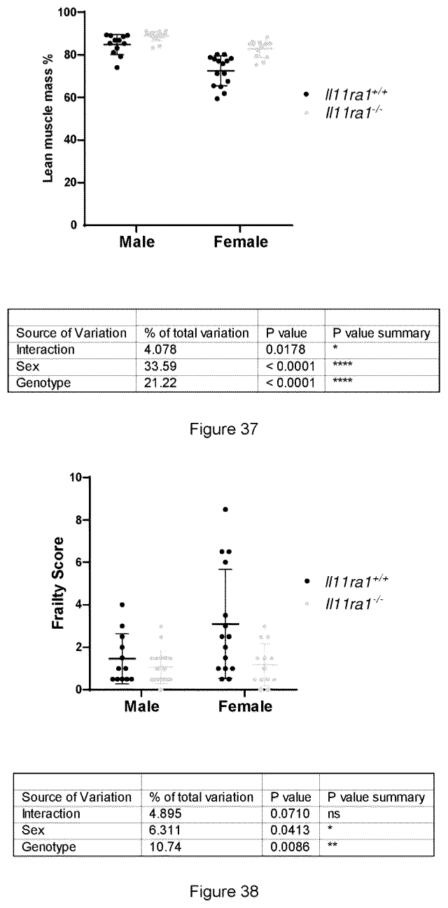

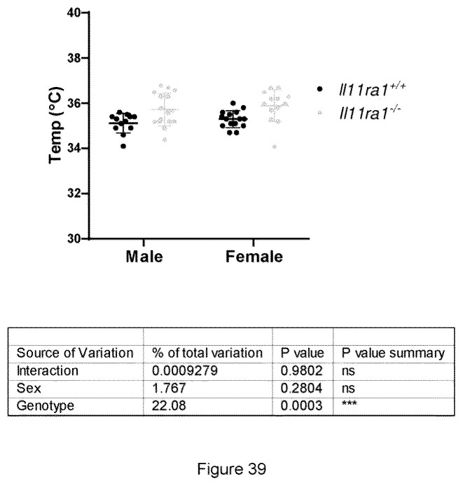

Phenotypic studies revealed that old Il11ra1-KO mice had less body weight (FIG. 35), less body fat percentage (FIG. 36) and more lean muscle mass percentage (FIG. 37) as compared to wild-type controls. Moreover, old Il11ra1-KO mice exhibited significantly reduced frailty (FIG. 38) and higher body temperature (FIG. 39) as compared to wild-type controls.

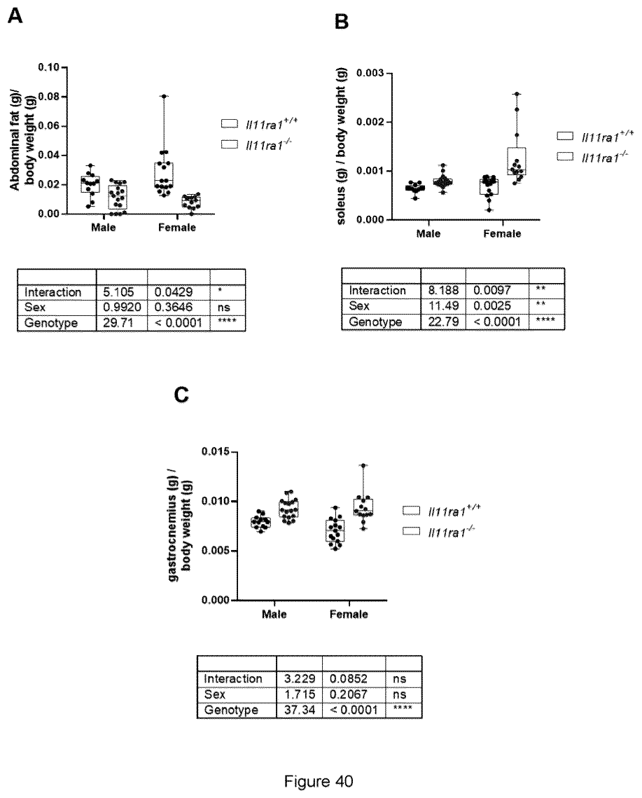

Deletion of Il11ra1 further led to old mice having less abdominal fat and more muscle (soleus and gastrocnemius) as compared to wild-type controls (FIGS. 40A, 40B and 40C).

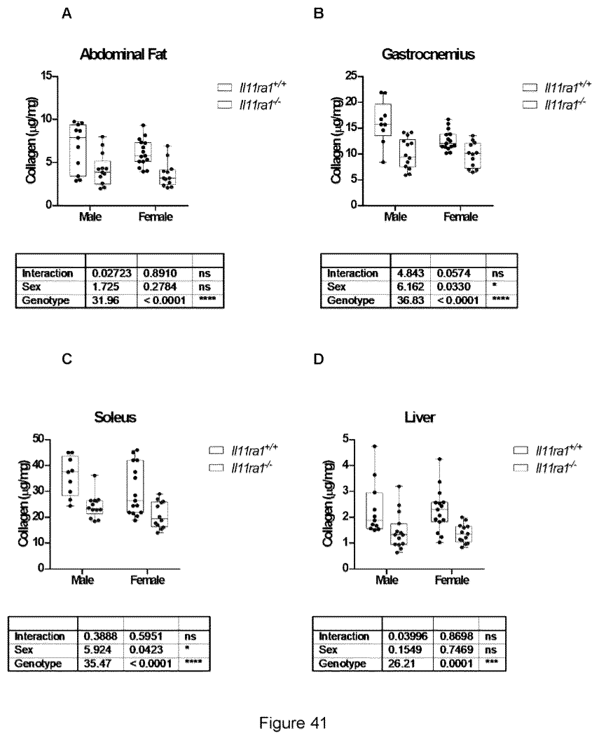

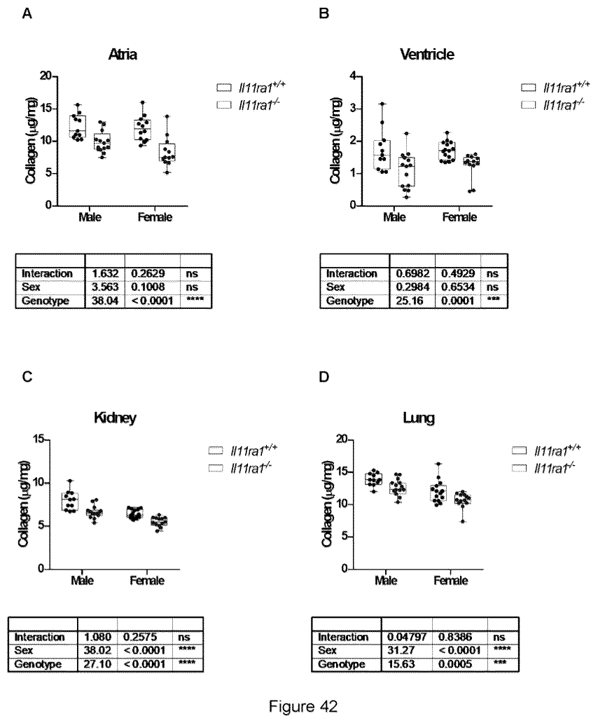

Fibrosis of abdominal fat, gastrocnemius, soleus, liver, heart atria, heart ventricle, kidney and lung was evaluated based on the collagen content in old Il11ra1-KO mice as compared to wild-type controls (FIGS. 41A to 41D and FIGS. 42A to 42D). Old Il11ra1 KO mice had less fibrosis across these tissues.

Example 6 demonstrates that mice deleted for Il11ra1 at 110 weeks old mirror the phenotype observed with administration of X203 (in Example 1 and FIGS. 1, 2, 3, 4A and 4B), namely improved multi-morbidity with better serum levels of fats, glucose, lesser tissue fibrosis, better kidney function, reduced tissue inflammation, reduced obesity and lesser frailty. It further demonstrates that Il11ra1-KO mice have an ageing signalling profile more similar to young mice, across tissues.

### Example 7: Effect of IL-11 Antagonism (X203) on Ageing Pathways In Vivo

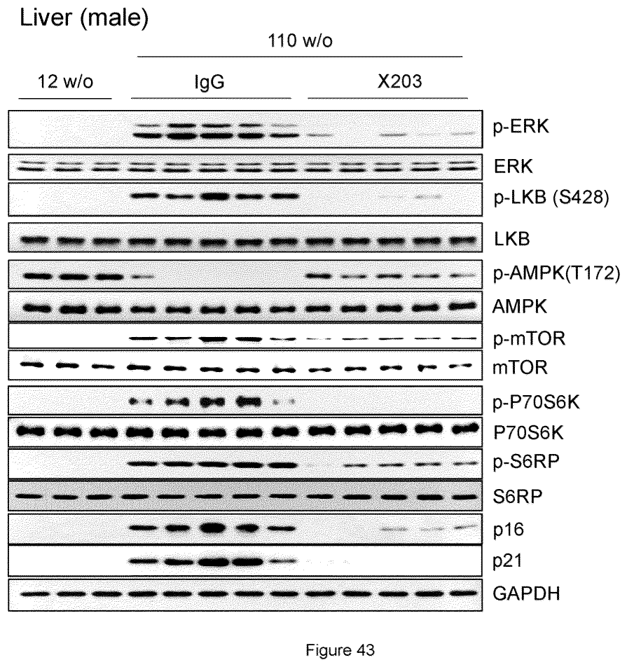

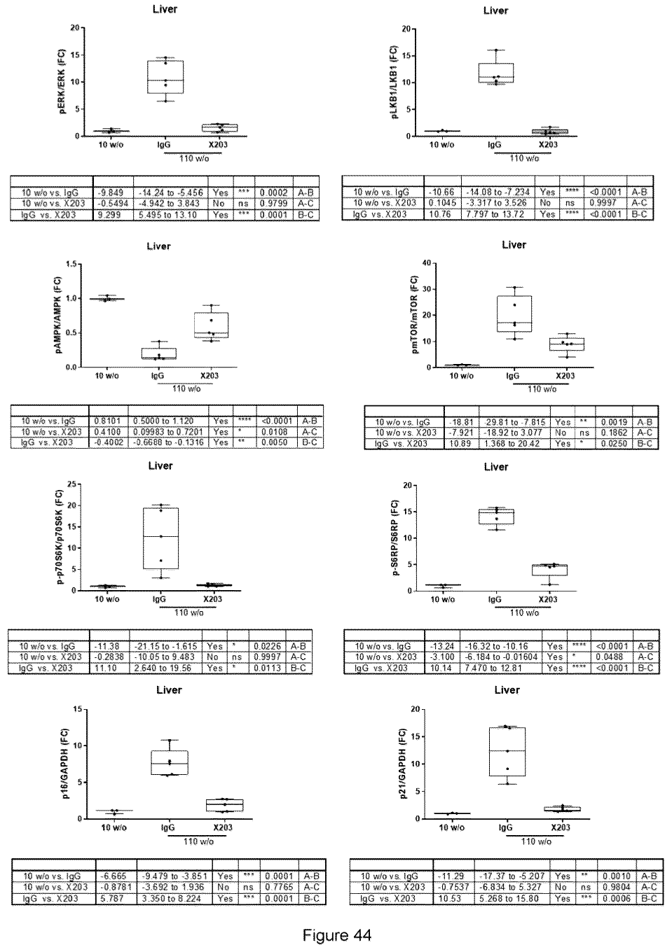

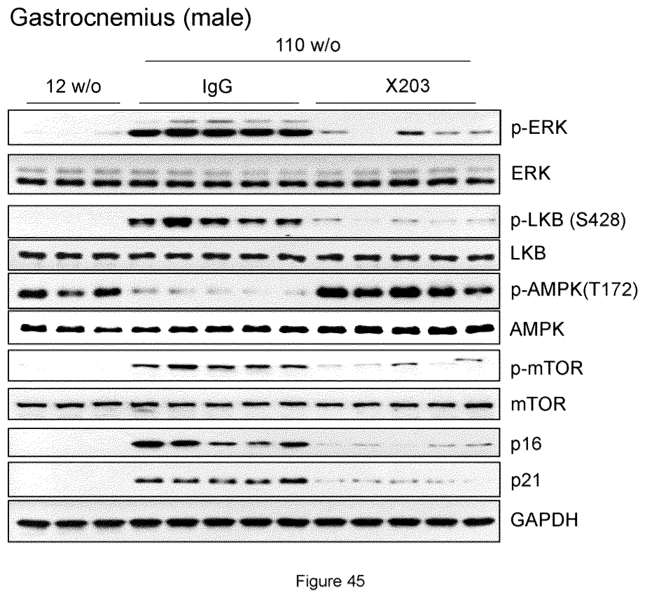

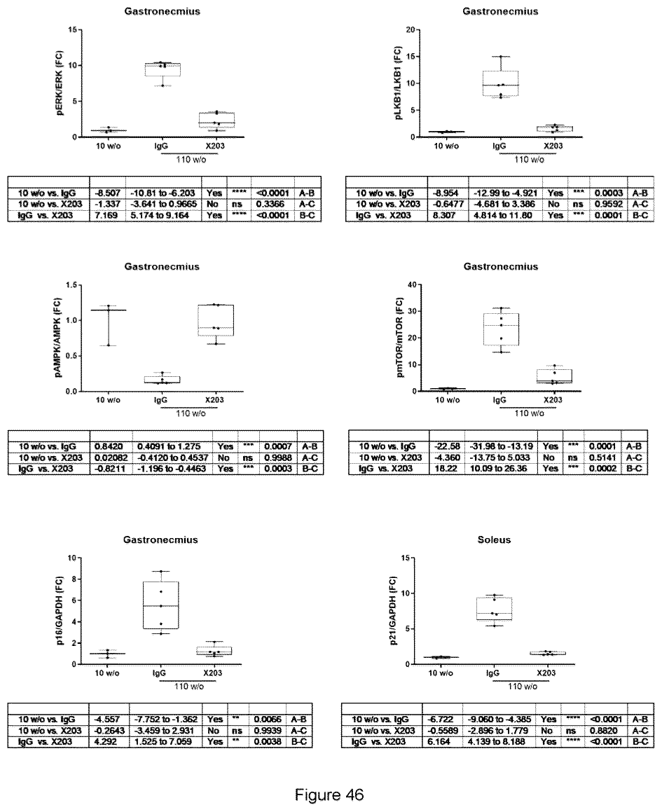

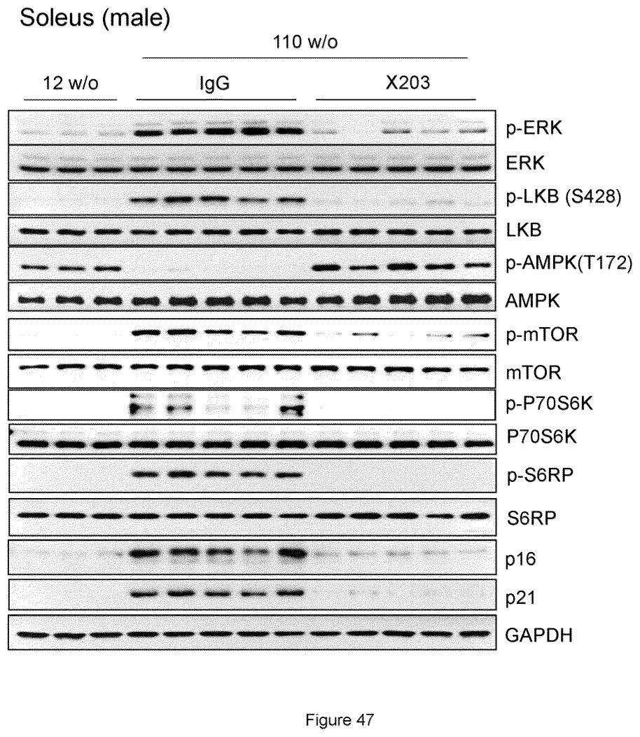

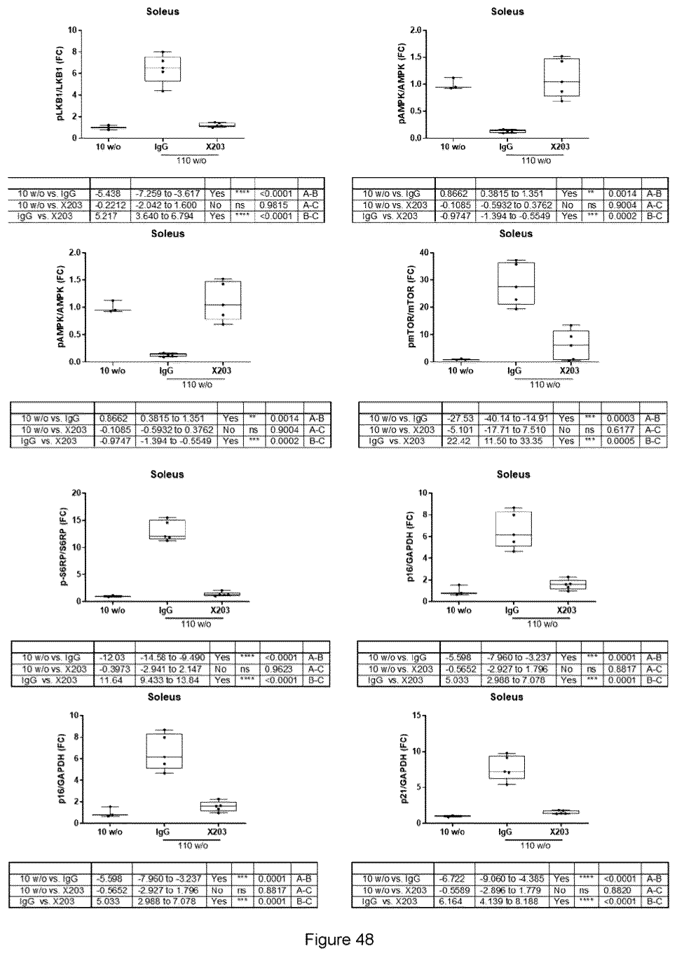

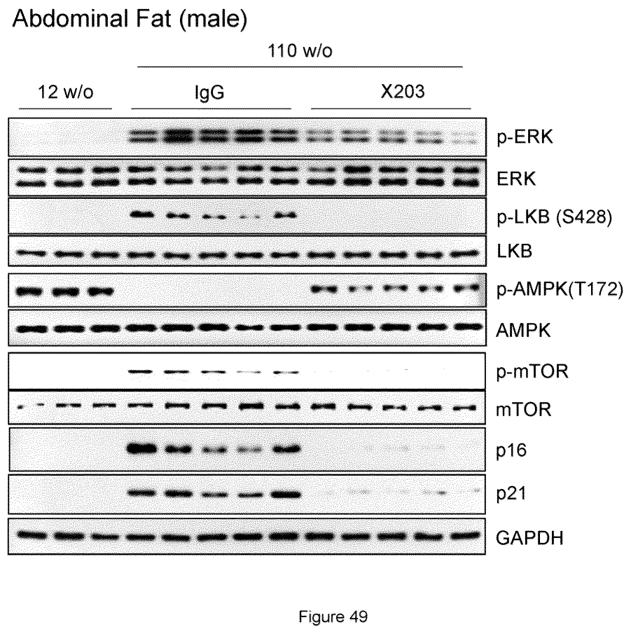

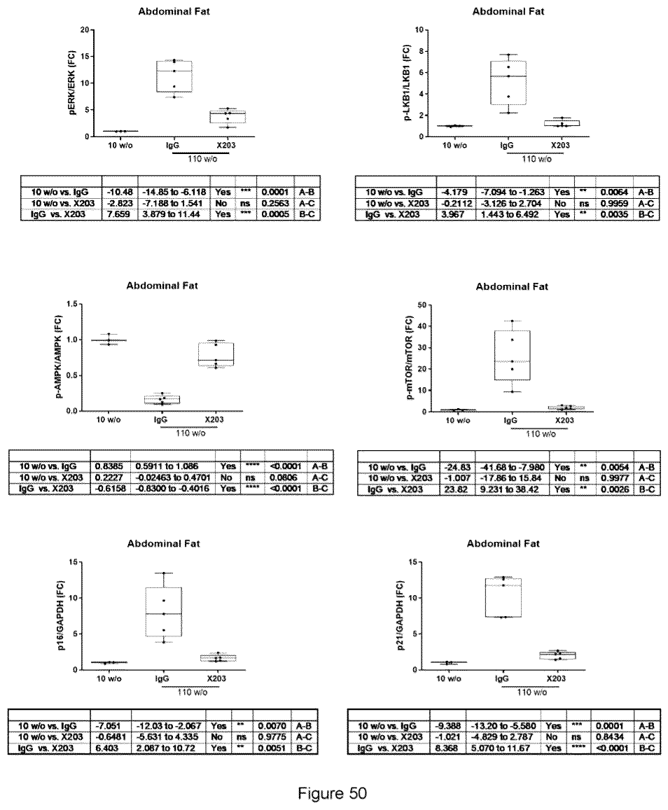

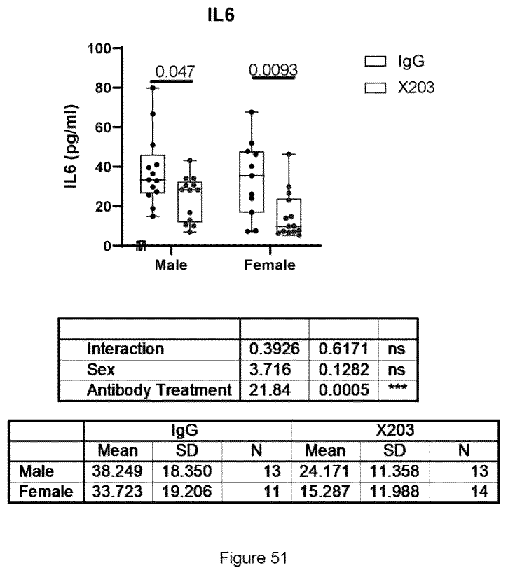

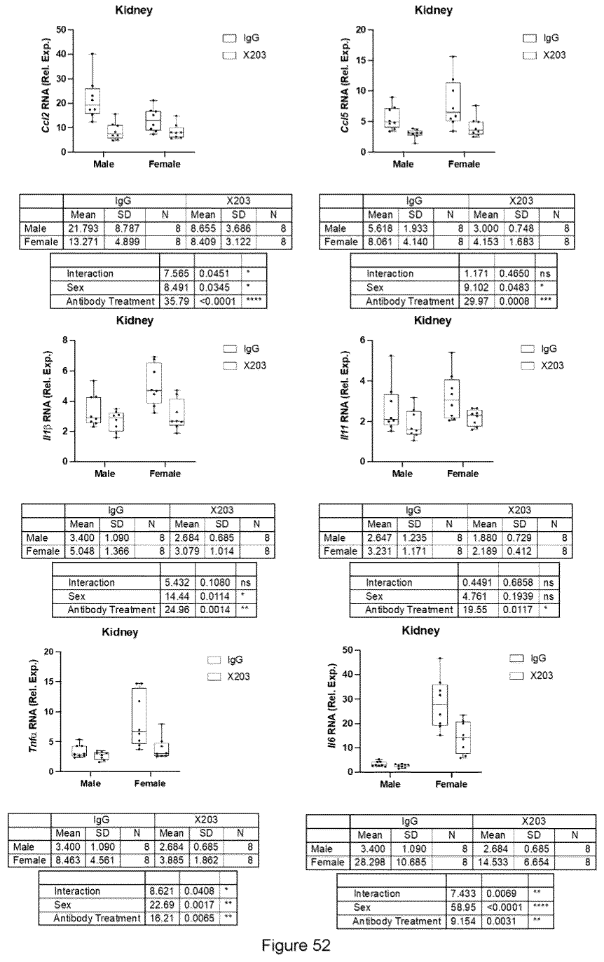

FIGS. 43 and 44 demonstrate that livers of 110-week (old) mice receiving a neutralising IL-11 antibody (X203) have less ERK/LKB1/mTORC1 activity, more AMPK activity, and less expression of key senescence markers (p16 and p21) as compared to old mice receiving the IgG control and young (12-week old) mice. The same results are observed in the gastrocnemii (FIGS. 45 and 46), solei (FIGS. 47 and 48) and abdominal fat (FIGS. 49 and 50) of old mice treated with X203.

In summary, a neutralizing IL-11 antibody restores LKB1/AMPK activity, reduces mTORC1 activity and inhibits expression of the key senescence markers p16 and p21 in livers, skeletal muscles (solei and gastrocnemii) and abdominal fat of old mice.

### Example 8: Effect of IL-11 Antagonism (X203) on Inflammatory Markers In Vivo

Altered intercellular communication, particularly increased chronic inflammation and IL-6 upregulation, is an important Hallmark of ageing that may be targeted to prevent, treat and/or reverse ageing processes (Furman et al. 2019, Nature Medicine 25 (12): 1822-32; Ferrucci and Fabbri 2018, Nature Reviews. Cardiology 15 (9): 505-22; Ershler and Keller 2000, Annual Review of Medicine 51: 245-70).

### 8.1 Effect of X203 on Serum Levels of IL-6

IL-6 is strongly linked with ageing (Maggio et al. 2006, J Gerontol A Biol Sci Med Sci. 61(6): 575-584; Ershler and Keller 2000, Annual Review of Medicine 51: 245-70). FIG. 50 shows the effects of treatment with anti-IL11 (X203) or IgG isotype control on serum IL-6 levels of old (110 week-old) male and female mice. X203 neutralizing IL-11 antibody significantly reduces the levels of IL-6 protein in the serum of old mice.

### 8.2 Effect of X203 on Inflammatory Markers in Liver, Kidney and Muscle

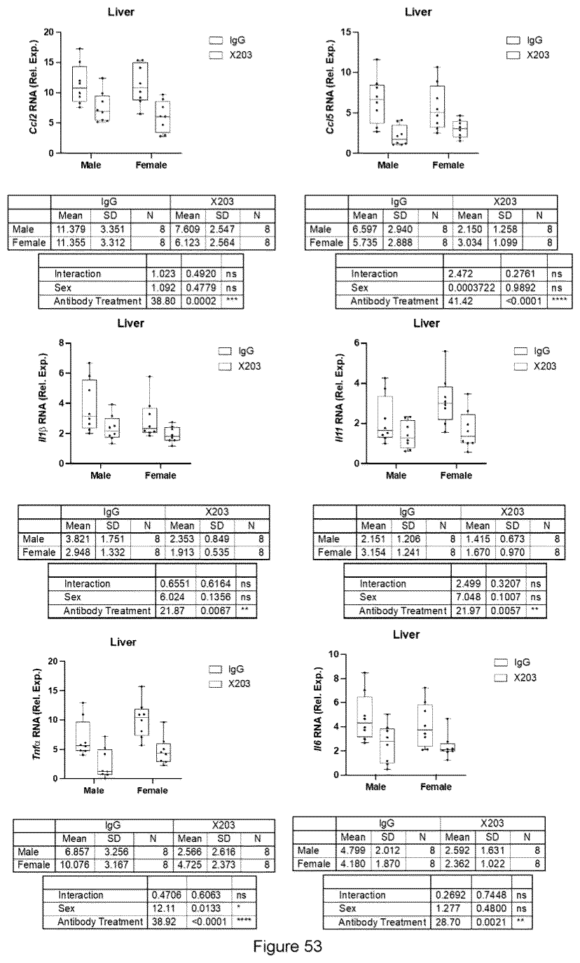

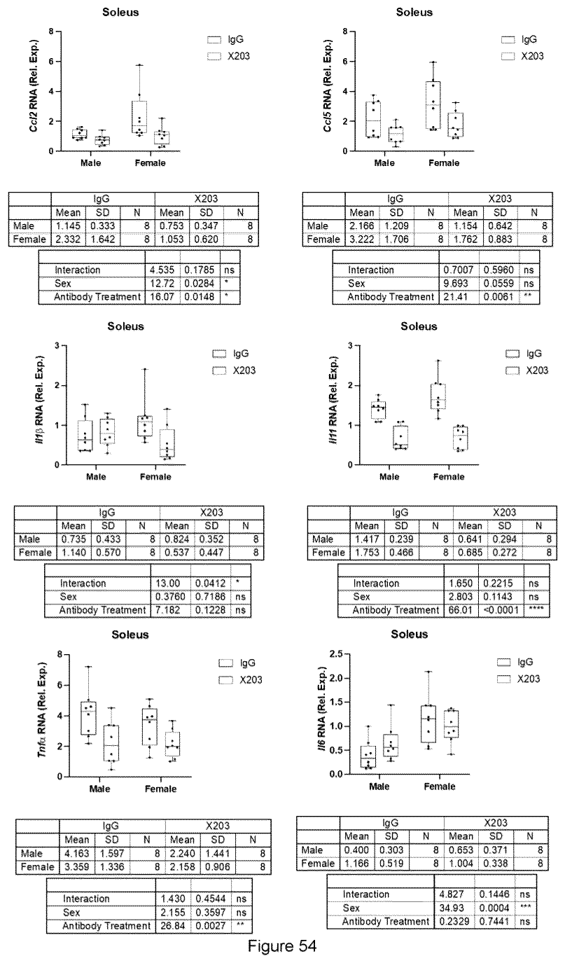

Administration of a neutralising anti-IL-11 (X203) vs IgG to old mice reduces markers of inflammation (CCL2, CCL5, TNFa, IL-1 b, IL-11 and IL-6) which correspond to an altered intercellular communication Hallmark of ageing across tissues, including liver (FIG. 52), kidney (FIG. 53) and skeletal muscle (FIG. 54). Effects on IL-6 are also noticeable in the liver and kidney at qPCR levels.

In summary, the data disclosed herein demonstrate that antagonism of IL-11 mediated signalling (e.g. by anti-IL-11 therapy) reduces multiple Hallmarks of Ageing: nutrient sensing, loss of proteostasis, cellular senescence and altered intercellular communication (inflammation). This is achieved by simultaneously targeting multiple ageing modules in parallel: ERK, AMPK and mTORC1.

Furthermore, the data disclose that:
Administration of MEK1/2 inhibitor U0126 to cells stimulated with IL11 prevents ERK-mediated inactivation of LKB1/AMPK that stops IL11-induced mTORC1/P70S6K activation.
Administration of anti-IL11 (X203) vs IgG control to mice aged 55-110 weeks old results in lesser ERK activation, restored LKB1/AMPK activity and reduced mTORC1 activity and senescence markers (p16/p21). This is seen across tissues.
Mice deleted for IL11RA1 at 110 weeks old mirror the effects seen with X203 administration and have a signaling profile more similar to young mice, across tissues.
Administration of X203 vs IgG to old mice improves multi-morbidity with better serum levels of fats, glucose, lesser tissue fibrosis, better kidney function, reduced tissue inflammation, reduced obesity and lesser frailty.
Administration of X203 vs IgG to old mice improves markers of inflammation (CCL2, CCL5, TNFa, IL1b, IL11 and IL6)—an altered intercellular communication Hallmark of ageing—across tissues, including liver, kidney and skeletal muscle. Effects on IL6, strongly linked with ageing, are noticeable in liver and kidney at QPCR levels and IL6 levels are systematically lower in the serum of old mice following anti-IL11 administration (Maggio et al. 2006; Ershler and Keller 2000)
In 110 weeks old Il11ra1 deleted mice, as compared to littermate controls, there is reduced multi-morbidity with better serum levels of fats, glucose, lesser tissue fibrosis, better kidney function, reduced tissue inflammation, reduced obesity and lesser frailty.
In in vitro models of senescence (AML12 cells), anti-IL11 or anti-IL11RA prevents and also reverses cellular senescence.
Treatment of mice with anti-IL11 from 55 to 110 weeks, as compared to controls, is associated with statistically significantly lesser frailty, using an accepted frailty scoring system (Sukoff Rizzo et al. 2018).
IL11RA1 KO mice, as compared to wildtype littermate controls, have statistically significantly lesser frailty, using an accepted frailty scoring system (Sukoff Rizzo et al 2018).

### Example 9: Materials and Methods Used in Examples 5 to 8

### Chemicals

Hydrogen Peroxide (H₂O₂, 31642, Sigma), palmitate (P5585, Sigma), rapamycin (9904, CST), U0126 (9903, CST) U0126 is a highly selective inhibitor of both MEK1 and MEK2, a type of MAPK/ERK kinase, wortmannin (9951, CST).

### Cell Culture

Cells were grown and maintained at 37° C. and 5% CO₂. The growth medium was renewed every 2-3 days and cells were passaged at 80% confluence, using standard trypsinization techniques. All experiments were carried out at low cell passage (<P3). Cells were serum-starved overnight in basal media prior to stimulation. Cells were stimulated with different treatment conditions and durations, as outlined in the figure legends. Stimulated cells were compared to unstimulated cells that have been grown for the same duration under the same conditions, but without the stimuli.

### Primary Human Cardiac Fibroblasts (HCFs)

Primary HCFs (6330, ScienCell) were grown and maintained in FM-2 complete media which contains Fibroblast medium-2 (2331, ScienCell), Fibroblasts growth supplement-2 (FGS-2, 2382, ScienCell), 5% fetal bovine serum, and 1% Penicillin-streptomycin (P/S, 0353, ScienCell).

### Primary Human Hepatic Stellate Cells (HSCs)

HSCs (5300, ScienCell) were cultured in stellate cells complete media (5301, ScienCell) on poly-L-lysine-coated plates (2 μg/cm², 0403, ScienCell).

### Primary Human Hepatocytes

Primary human hepatocytes (5200, ScienCell) were maintained in hepatocyte medium (5201, ScienCell) supplemented with 2% fetal bovine serum, 1% Penicillin-streptomycin at 37° C. and 5% CO₂.

### AML12

AML12 (ATCC) were cultured in DMEM:F12 Medium (30-2006, ATCC) supplemented with 10% FBS, 10 μg/ml insulin, 5.5 μg/ml transferrin, 5 ng/ml selenium, and 40 ng/ml dexamethasone.

### Senescence Induction

Senescent AML12 were generated by treating the cells with a sub-lethal concentration of H₂O₂ (0.75 mM) for 1 hour, followed by 23 hours recovery per day, for 7 days as described (Tripathi, Yen, and Singh 2020, STAR Protocols 1 (2): 100064). For prevention study, AML12 cells were treated with 2 ug/ml of either IgG or X209 during the recovery period of the senescence induction stage (from day 1 to day 7); cells were harvested for total RNA collection at day 10. For reversal study, senescence was first induced for seven days followed by 2 ug/ml of IgG, X203, or X209 treatment for 3 days; cells were harvested for total RNA collection at day 10.

### Animal Models

### In Vivo Administration of Anti-IL11 (X203)

Male and female C57Bl/6J mice aged 46 weeks were purchased from the Jackson Laboratory. Mice were randomised on a 1:1 basis to receive either 40 mg/kg of X203 or an isotype control IgG antibody (11E10), every 3 weeks (IP), starting from 55 weeks of age. Mice were sacrificed at 110 weeks of age for blood and tissues collection. 12 weeks old male and female C57Bl/6J mice that were used as control were purchased from Invivos (Singapore).

### Il11ra1-Deleted Mice (Il11ra1 KO, B6.129S1-Il11ra^{tm1Wehi}/J, Jackson's Laboratory)

Male and female Il11ra1^{+/+} (wild-type) and Il11ra1^{-/-} mice were sacrificed at 110 weeks of age for blood and tissues collection; 10-12 weeks old male and female mice of the respective genotypes were used as controls.

### Echo MRI Analysis for Body Composition

End-point total body fat and lean mass measurements were performed 2 days prior to sacrifice by EchoMRI analysis using 4in1 Body Composition Analyzer for Live Small Animal.

### Frailty Scoring

End-point frailty scoring was performed 2 days prior to sacrifice using the accepted frailty scoring system (Sukoff Rizzo et al. 2018, Current Protocols in Mouse Biology 8 (2): e45.)

### Immunoblotting

Western blots were carried out on total protein extracts from primary human cardiac fibroblasts (HCFs), hepatic stellate cells (HSCs), hepatocytes, livers, gastrocnemii, solei, and abdominal fats. Cell or tissue lysates were homogenized in RIPA Lysis and Extraction Buffer (89901, Thermo Scientific) containing protease and phosphatase inhibitors (Roche). Protein lysates were separated by SDS-PAGE, transferred to PVDF membranes, blocked for 1 h with 3% BSA, and incubated overnight with either phospho-ACC (11818, CST), ACC (3676, CST), phospho-AMPK (2535, CST), AMPK (5832, CST), phospho-ERK1/2 (4370, CST), ERK1/2 (4695, CST), GAPDH (2118, CST), phospho-LKB1 S428 (3482, CST), phospho-LKB1 (S325), LKB1 (3047, CST), (phospho-mTOR (2971, CST), mTOR (2972, CST), p16 (ab232402, Abcam), p21 (64016, CST), phospho-p70S6K (9205, CST), p70S6K (2708, CST), phospho-S6 ribosomal protein (4858, CST), or S6 ribosomal protein (2217, CST). All primary antibodies are diluted 1:1000 in 1% BSA. Protein bands were visualized using the ECL detection system (Pierce) with anti-rabbit HRP (1:2000 in 3% BSA, 7074, CST).

### Hydroxyproline Assay

Total hydroxyproline content in mouse livers was measured using Quickzyme Total Collagen assay kit (QZBtotco15, Quickzyme Biosciences).

### Enzyme-Linked Immunosorbent Assay (ELISA)

The levels of IL6 in mouse serum were quantified using Mouse IL-6 Quantikine ELISA Kit (M6000B; R&D Systems).

### RT-qPCR

Total RNA was extracted from AML12 or snap-frozen tissues using Trizol (Invitrogen) and RNeasy Mini Kit (Qiagen). PCR amplifications were performed using iScript cDNA Synthesis Kit (Biorad). Gene expression analysis was performed on duplicate samples with either TaqMan (Applied Biosystems) for Il11 (Mm00434162) or fast SYBR green (Qiagen) technology using StepOnePlus™ (Applied Biosystem) for Ccl2, Ccl5, Gapdh, Il1β, Il6, Il8, p16, p21, Tnfα, over 40 cycles. Expression data were normalized to GAPDH mRNA expression and fold change was calculated using 2^{-ΔΔct} method.

List of primer sequences

| Host | Gene | Forward (5′-3′) | Reverse (5′-3′) |
|---|---|---|---|
| Mouse | Tnfa | ATGAGAAGTTCCCAAATGGC | CTCCACTTGGTGGTTTGCTA |
| | Ccl2 | GAAGGAATGGGTCCAGACAT | ACGGGTCAACTTCACATTCA |
| | Ccl5 | GCTGCTTTGCCTACCTCTCC | TCGAGTGACAAACACGACTG |
| | | | C |
| | ll6 | CTCTGGGAAATCGTGGAAAT | CCAGTTTGGTAGCATCCATC |
| | ll8 | ACTGAGAGTGATTGAGAGTG | AACCCTCTGCACCCAGTTTT |
| | | GAC | C |
| | ll1β | CACAGCAGCACATCAACAAG | GTGCTCATGTCCTCATCCTG |
| | p21 | GTACTTCCTCTGCCCTGCTG | TTTCGGCCCTGAGATGTTCC |
| | p16 | GGGTTTCGCCCAACGCCCCG | TGCAGCACCACCAGCGTGTC |
| | | A | C |
| | Gapdh | CTGGAAAGCTGTGGCGTGAT | GACGGACACATTGGGGGTAG |

### Immunofluorescence Staining

Kidneys, hearts, lungs, and spleens from 110 week-old of wild-type and IL11:EGFP reporter mice (Il11-Egfp^{+/-}; (Widjaja et al. 2021, Science Translational Medicine 13 (597)) were harvested, fixed in 4% paraformaldehyde, dehydrated in 15% and 30% sucrose, and embedded in OCT cryoblocks. Sections (7 μm) were prepared using standard methodology by fixation in methanol:acetone (1:1) and 0.1% Triton-X100 combined with mouse-on-mouse blocking (MKB-2213-1, Vector Labs) and 5% normal goat serum. Kidney sections were stained for EGFP (GFP, ab290, Abcam (1:500)) and αSMA (αSMA, ab7817, Abcam (1:200)); heart (atrium and ventricle) sections were stained for EGFP (GFP, sc-9966, Santacruz (1:100)) and αSMA (αSMA, ab5694, Abcam (1:500)); spleen sections were stained for EGFP (GFP, sc-9966, Santacruz (1:100)). Tissue sections were then incubated with the appropriate secondary antibodies conjugated to fluorophores: goat anti-rabbit IgG AF647 (A27040, ThermoFisher Scientific (1:500)) and goat anti-mouse IgG AF555 (A28180, ThermoFisher Scientific (1:500)) followed by autofluorescence quenching by 0.1% Sudan Black B in 70% ethanol.

### REFERENCES

The following citations are incorporated by reference in their entirety.
Burkewitz, Kristopher, Yue Zhang, and William B. Mair. 2014. “AMPK at the Nexus of Energetics and Aging.” Cell Metabolism 20 (1): 10-25.
Dolgin, Elie. 2020. “Send in the Senolytics.” Nature Biotechnology 38 (12): 1371-77.
Ershler, W. B., and E. T. Keller. 2000. “Age-Associated Increased Interleukin-6 Gene Expression, Late-Life Diseases, and Frailty.” Annual Review of Medicine 51: 245-70.
Fernandes, Stephanie A., and Constantinos Demetriades. 2021. “The Multifaceted Role of Nutrient Sensing and mTORC1 Signaling in Physiology and Aging.” Frontiers in Aging 2: 38.
Ferrucci, Luigi, and Elisa Fabbri. 2018. “Inflammageing: Chronic Inflammation in Ageing, Cardiovascular Disease, and Frailty.” Nature Reviews. Cardiology 15 (9): 505-22.
Furman, David, Judith Campisi, Eric Verdin, Pedro Carrera-Bastos, Sasha Targ, Claudio Franceschi,
Luigi Ferrucci, et al. 2019. “Chronic Inflammation in the Etiology of Disease across the Life Span.” Nature Medicine 25 (12): 1822-32.
Kaushik, Susmita, and Ana Maria Cuervo. 2015. “Proteostasis and Aging.” Nature Medicine 21 (12): 1406-15.
Liu, Grace Y., and David M. Sabatini. 2020. “mTOR at the Nexus of Nutrition, Growth, Ageing and Disease.” Nature Reviews, Molecular Cell Biology 21 (4): 183-203.
López-Otín, Carlos, Maria A. Blasco, Linda Partridge, Manuel Serrano, and Guido Kroemer. 2013. “The Hallmarks of Aging.” Cell 153 (6): 1194-1217.
Maggio, Marcello, Jack M. Guralnik, Dan L. Longo, and Luigi Ferrucci. 2006. “Interleukin-6 in Aging and Chronic Disease: A Magnificent Pathway.” The Journals of Gerontology. Series A, Biological Sciences and Medical Sciences 61 (6): 575-84.
Shaw, Reuben J., Katja A. Lamia, Debbie Vasquez, Seung-Hoi Koo, Nabeel Bardeesy, Ronald A. Depinho, Marc Montminy, and Lewis C. Cantley. 2005. “The Kinase LKB1 Mediates Glucose Homeostasis in Liver and Therapeutic Effects of Metformin.” Science 310 (5754): 1642-46.
Slack, Cathy, Nazif Alic, Andrea Foley, Melissa Cabecinha, Matthew P. Hoddinott, and Linda Partridge. 2015. “The Ras-Erk-ETS-Signaling Pathway Is a Drug Target for Longevity.” Cell 162 (1): 72-83.
Sukoff Rizzo, Stacey J., Laura C. Anderson, Torrian L. Green, Tracy McGarr, Gaylynn Wells, and Shawn S. Winter. 2018. “Assessing Healthspan and Lifespan Measures in Aging Mice: Optimization of Testing Protocols, Replicability, and Rater Reliability.” Current Protocols in Mouse Biology 8 (2): e45.
Tripathi, Madhulika, Paul Michael Yen, and Brijesh Kumar Singh. 2020. “Protocol to Generate Senescent Cells from the Mouse Hepatic Cell Line AML12 to Study Hepatic Aging.” STAR Protocols 1 (2): 100064.
Widjaja, Anissa A., Jinrui Dong, Eleonora Adami, Sivakumar Viswanathan, Benjamin Nq, Leroy S. Pakkiri, Sonia P. Chothani, et al. 2021. “Redefining IL11 as a Regeneration-Limiting Hepatotoxin and Therapeutic Target in Acetaminophen-Induced Liver Injury.” Science Translational Medicine 13 (597). https://doi.org/10.1126/scitranslmed.aba8146.
Zhang, Yan, Jinjin Zhang, and Shixuan Wang. 2021. “The Role of Rapamycin in Healthspan Extension via the Delay of Organ Aging.” Ageing Research Reviews 70 (September): 101376.

1. A method of treating or preventing an age-related disease/condition, comprising administering a therapeutically or prophylactically effective amount of an agent capable of inhibiting interleukin 11 (IL-11)-mediated signalling to a subject, wherein the age-related disease/condition is selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

2. An agent capable of inhibiting interleukin 11 (IL-11)-mediated signalling for use in a method of treating or preventing an age-related disease/condition is selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related cardiovascular disease, age-related hypertension, age-related renal disease and age-related skin disease.

3. Use of an agent capable of inhibiting interleukin 11 (IL-11)-mediated signalling in the manufacture of a medicament for use in a method of treating or preventing an age-related disease/condition selected from: frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

4. An agent capable of inhibiting interleukin 11 (IL-11)-mediated signalling for use in a method of treating or preventing frailty.

5. Use of an agent capable of inhibiting interleukin 11 (IL-11)-mediated signalling in the manufacture of a medicament for use in a method of treating or preventing frailty.

6. A method of treating or preventing frailty, comprising administering a therapeutically or prophylactically effective amount of an agent capable of inhibiting interleukin 11 (IL-11)-mediated signalling to a subject.

7. An agent capable of inhibiting interleukin 11 (IL-11)-mediated signalling for use in a method of treating or preventing an age-related change in body composition.

8. Use of an agent capable of inhibiting interleukin 11 (IL-11)-mediated signalling in the manufacture of a medicament for use in a method of treating or preventing an age-related change in body composition.

9. A method of treating or preventing an age-related change in body composition, comprising administering a therapeutically or prophylactically effective amount of an agent capable of inhibiting interleukin 11 (IL-11)-mediated signalling to a subject.

10. An agent capable of inhibiting interleukin 11 (IL-11)-mediated signalling for use in a method of increasing the healthspan of a subject.

11. Use of an agent capable of inhibiting interleukin 11 (IL-11)-mediated signalling in the manufacture of a medicament for use in a method of increasing the healthspan of a subject.

12. A method of increasing the healthspan of a subject, comprising administering a therapeutically or prophylactically effective amount of an agent capable of inhibiting interleukin 11 (IL-11)-mediated signalling to the subject.

13. The agent for use according to any one of claims 1, 4, 7 or 10, the use according to any one of claims claim 2, 5, 8 or 11, or the method according to any one of claims 3, 6, 9 or 12, wherein the agent is selected from the group consisting of: an antibody or an antigen-binding fragment thereof, a polypeptide, a peptide, a nucleic acid, an oligonucleotide, an aptamer or a small molecule.

14. The agent for use, the use or the method according to any one of claims 1 to 13, wherein the agent is an agent capable of preventing or reducing the binding of interleukin 11 (IL-11) to a receptor for interleukin 11 (IL-11R).

15. The agent for use, the use or the method according to any one of claims 1 to 14, wherein the agent is capable of binding to interleukin 11 (IL-11) or a receptor for interleukin 11 (IL-11R).

16. The agent for use, the use or the method according to any one of claims 1 to 15, wherein the agent is an antibody or an antigen-binding fragment thereof.

17. The agent for use, the use or the method according to any one of claims 1 to 16, wherein the agent is an anti-IL-11 antibody antagonist of IL-11-mediated signalling, or an antigen-binding fragment thereof.

18. The agent for use, the use or the method according to any one of claims 1 to 16, wherein the agent is an anti-IL-11Rα antibody antagonist of IL-11-mediated signalling, or an antigen-binding fragment thereof.

19. The agent for use, the use or the method according to any one of claims 1 to 15, wherein the agent is a decoy receptor for IL-11.

20. The agent for use, the use or the method according to any one of claims 1 to 15, wherein the agent is a competitive inhibitor of IL-11.

21. The agent for use, the use or the method according to any one of claims 1 to 13, wherein the agent is capable of preventing or reducing the expression of interleukin 11 (IL-11) or a receptor for interleukin 11 (IL-11R).

22. The agent for use, the use or the method according to claim 21, wherein the agent is an antisense oligonucleotide capable of preventing or reducing the expression of IL-11.

23. The agent for use, the use or the method according to claim 21, wherein the agent is an antisense oligonucleotide capable of preventing or reducing the expression of IL-11Rα.

24. The agent for use, the use or the method according to any one of claims 1 to 23, wherein the method comprises administering the agent to a subject in which expression of interleukin 11 (IL-11) or a receptor for IL-11 (IL-11R) is upregulated.


SEQUENCE LISTING (78 sequences)

SEQ ID NO: 1 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 2 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 3 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 4 - DNA, 19 bp, Artificial Sequence
  source: /note=“Description of Artificial Sequence Synthetic oligonucleotide”
ccttccaaagccagatctt

SEQ ID NO: 5 - DNA, 19 bp, Artificial Sequence
  source: /note=“Description of Artificial Sequence Synthetic oligonucleotide”
gcctgggcaggaacatata

SEQ ID NO: 6 - DNA, 19 bp, Artificial Sequence
  source: /note=“Description of Artificial Sequence Synthetic oligonucleotide”
cctgggcaggaacatatat

SEQ ID NO: 7 - DNA, 19 bp, Artificial Sequence
  source: /note=“Description of Artificial Sequence Synthetic oligonucleotide”
ggttcattatggctgtgtt

SEQ ID NO: 8 - DNA, 19 bp, Artificial Sequence
  source: /note=“Description of Artificial Sequence Synthetic oligonucleotide”
ggaccataccaaaggagat

SEQ ID NO: 9 - DNA, 19 bp, Artificial Sequence
  source: /note=“Description of Artificial Sequence Synthetic oligonucleotide”
gcgtctttgggaatccttt

SEQ ID NO: 10 - DNA, 18 bp, Artificial Sequence
  source: /note=“Description of Artificial Sequence Synthetic oligonucleotide”
gcaggacagtagatccct

SEQ ID NO: 11 - DNA, 19 bp, Artificial Sequence
  source: /note=“Description of Artificial Sequence Synthetic oligonucleotide”
gctcaaggaacgtgtgtaa

SEQ ID NO: 12 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
ccuuccaaagccagaucuuttaagaucuggcuuuggaaggtt

SEQ ID NO: 13 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
gccugggcaggaacauauattuauauguuccugcccaggctt

SEQ ID NO: 14 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
ccugggcaggaacauauauttauauauguuccugcccaggtt

SEQ ID NO: 15 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
gguucauuauggcuguguuttaacacagccauaaugaacctt

SEQ ID NO: 16 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
ggaccauaccaaaggagauttaucuccuuugguauggucctt

SEQ ID NO: 17 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
gcgucuuugggaauccuuuttaaaggauucccaaagacgctt

SEQ ID NO: 18 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
gcaggacaguagaucccuattuagggaucuacuguccugctt

SEQ ID NO: 19 - DNA, 42 bp, Artificial Sequence
  source: /note=“Description of Artificial Sequence Synthetic oligonucleotide”
  source: /note=“Description of Combined DNA/RNA Molecule Synthetic oligonucleotide”
gcucaaggaacguguguaattuuacacacguuccuugagctt

SEQ ID NO: 20 - protein, 21 aa, Artificial Sequence
  source: /note=“Description of Artificial Sequence Synthetic peptide”
GPAGQSGGGGGSGGGSGGGSV

SEQ ID NO: 21 - protein, 516 aa, Artificial Sequence
  source: /note=“Description of Artificial Sequence Synthetic polypeptide”
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGPAGQSGGGGGSGGGSGGGSVPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 22 - protein, 119 aa, Artificial Sequence
  source: /note=“Description of Artificial Sequence Synthetic polypeptide”
EVQLQQSGPELVKPGASVKIPCKASGYTFTDYNMDWVKQSHGKSLEWIGDINPHNGGPIYNQKFTGKATLTVDKSSSTAYMELRSLTSEDTAVYYCARGELGHWYFDVWGTGTTVTVSS

SEQ ID NO: 23 - protein, 111 aa, Artificial Sequence
  source: /note=“Description of Artificial Sequence Synthetic polypeptide”
DIVLTQSPASLAVSLGQRATISCRASKSVSTSGYSYIHWYQQKPGQPPKLLIYLASNLDSGVPARFSGSGSGTDFTLNIHPVEEEDAATYYCQHSRDLPPTFGGGTKLEIK

SEQ ID NO: 24 - protein, 117 aa, Artificial Sequence
  source: /note=“Description of Artificial Sequence Synthetic polypeptide”
QVQLQQPGAELVRPGSSVKLSCKASGYTFTNYWMHWLKQRPVQGLEWIGNIGPSDSKTHYNQKFKDKATLTVDKSSSTAYMQLNSLTSEDSAVYYCARGDYVLFTYWGQGTLVTVSA

SEQ ID NO: 25 - protein, 107 aa, Artificial Sequence
  source: /note=“Description of Artificial Sequence Synthetic polypeptide”
DIVLTQSPATLSLSPGERATLSCRASQSISNNLHWYQQKSHEAPRLLIKYASQSISGIPARFSGSGSGTDFTLSFSSLETEDFAVYFCQQSYSWPLTFGQGTKLEIK

SEQ ID NO: 26 - protein, 118 aa, Artificial Sequence
  source: /note=“Description of Artificial Sequence Synthetic polypeptide”
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSS

SEQ ID NO: 27 - protein, 111 aa, Artificial Sequence
  source: /note=“Description of Artificial Sequence Synthetic polypeptide”
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLG

SEQ ID NO: 28 - protein, 445 aa, Artificial Sequence
  source: /note=“Description of Artificial Sequence Synthetic polypeptide”
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 216 aa, Artificial Sequence
  source: /note=“Description of Artificial Sequence Synthetic polypeptide”
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 119 aa, Artificial Sequence
  source: /note=“Description of Artificial Sequence Synthetic polypeptide”
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 31 - protein, 111 aa, Artificial Sequence
  source: /note=“Description of Artificial Sequence Synthetic polypeptide”
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIK

SEQ ID NO: 32 - protein, 117 aa, Artificial Sequence
  source: /note=“Description of Artificial Sequence Synthetic polypeptide”
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSS

SEQ ID NO: 33 - protein, 107 aa, Artificial Sequence
  source: /note=“Description of Artificial Sequence Synthetic polypeptide”
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIK

SEQ ID NO: 34 - protein, 5 aa, Artificial Sequence
  source: /note=“Description of Artificial Sequence Synthetic peptide”
SYGMH

SEQ ID NO: 35 - protein, 17 aa, Artificial Sequence
  source: /note=“Description of Artificial Sequence Synthetic peptide”
VISYDGSNKYYADSVKG

SEQ ID NO: 36 - protein, 9 aa, Artificial Sequence
  source: /note=“Description of Artificial Sequence Synthetic peptide”
IGATDPLDY

SEQ ID NO: 37 - protein, 14 aa, Artificial Sequence
  source: /note=“Description of Artificial Sequence Synthetic peptide”
TGTSSDVGGYNYVS

SEQ ID NO: 38 - protein, 7 aa, Artificial Sequence
  source: /note=“Description of Artificial Sequence Synthetic peptide”
DVNERSS

SEQ ID NO: 39 - protein, 10 aa, Artificial Sequence
  source: /note=“Description of Artificial Sequence Synthetic peptide”
ASYAGRYTWM

SEQ ID NO: 40 - protein, 5 aa, Artificial Sequence
  source: /note=“Description of Artificial Sequence Synthetic peptide”
DYNMD

SEQ ID NO: 41 - protein, 17 aa, Artificial Sequence
  source: /note=“Description of Artificial Sequence Synthetic peptide”
DINPHNGGPIYNQKFTG

SEQ ID NO: 42 - protein, 10 aa, Artificial Sequence
  source: /note=“Description of Artificial Sequence Synthetic peptide”
GELGHWYFDV

SEQ ID NO: 43 - protein, 15 aa, Artificial Sequence
  source: /note=“Description of Artificial Sequence Synthetic peptide”
RASKSVSTSGYSYIH

SEQ ID NO: 44 - protein, 7 aa, Artificial Sequence
  source: /note=“Description of Artificial Sequence Synthetic peptide”
LASNLDS

SEQ ID NO: 45 - protein, 9 aa, Artificial Sequence
  source: /note=“Description of Artificial Sequence Synthetic peptide”
QHSRDLPPT

SEQ ID NO: 46 - protein, 5 aa, Artificial Sequence
  source: /note=“Description of Artificial Sequence Synthetic peptide”
NYWMH

SEQ ID NO: 47 - protein, 17 aa, Artificial Sequence
  source: /note=“Description of Artificial Sequence Synthetic peptide”
NIGPSDSKTHYNQKFKD

SEQ ID NO: 48 - protein, 8 aa, Artificial Sequence
  source: /note=“Description of Artificial Sequence Synthetic peptide”
GDYVLFTY

SEQ ID NO: 49 - protein, 11 aa, Artificial Sequence
  source: /note=“Description of Artificial Sequence Synthetic peptide”
RASQSISNNLH

SEQ ID NO: 50 - protein, 7 aa, Artificial Sequence
  source: /note=“Description of Artificial Sequence Synthetic peptide”
YASQSIS

SEQ ID NO: 51 - protein, 9 aa, Artificial Sequence
  source: /note=“Description of Artificial Sequence Synthetic peptide”
QQSYSWPLT

SEQ ID NO: 52 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 107 aa, Homo sapiens [sequence omitted]

SEQ ID NO: 55 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 449 aa, Artificial Sequence
  source: /note=“Description of Artificial Sequence Synthetic polypeptide”
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 218 aa, Artificial Sequence
  source: /note=“Description of Artificial Sequence Synthetic polypeptide”
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 444 aa, Artificial Sequence
  source: /note=“Description of Artificial Sequence Synthetic polypeptide”
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, Artificial Sequence
  source: /note=“Description of Artificial Sequence Synthetic polypeptide”
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 5 aa, Artificial Sequence
  source: /note=“Description of Artificial Sequence Synthetic peptide”
  MOD_RES (3)..(3): Any amino acid
WSXWS

SEQ ID NO: 61 - DNA, 20 bp, Artificial Sequence
  source: /note=“Description of Artificial Sequence Synthetic primer”
atgagaagttcccaaatggc

SEQ ID NO: 62 - DNA, 20 bp, Artificial Sequence
  source: /note=“Description of Artificial Sequence Synthetic primer”
gaaggaatgggtccagacat

SEQ ID NO: 63 - DNA, 20 bp, Artificial Sequence
  source: /note=“Description of Artificial Sequence Synthetic primer”
gctgctttgcctacctctcc

SEQ ID NO: 64 - DNA, 20 bp, Artificial Sequence
  source: /note=“Description of Artificial Sequence Synthetic primer”
ctctgggaaatcgtggaaat

SEQ ID NO: 65 - DNA, 23 bp, Artificial Sequence
  source: /note=“Description of Artificial Sequence Synthetic primer”
actgagagtgattgagagtggac

SEQ ID NO: 66 - DNA, 20 bp, Artificial Sequence
  source: /note=“Description of Artificial Sequence Synthetic primer”
cacagcagcacatcaacaag

SEQ ID NO: 67 - DNA, 20 bp, Artificial Sequence
  source: /note=“Description of Artificial Sequence Synthetic primer”
gtacttcctctgccctgctg

SEQ ID NO: 68 - DNA, 21 bp, Artificial Sequence
  source: /note=“Description of Artificial Sequence Synthetic primer”
gggtttcgcccaacgccccga

SEQ ID NO: 69 - DNA, 20 bp, Artificial Sequence
  source: /note=“Description of Artificial Sequence Synthetic primer”
ctggaaagctgtggcgtgat

SEQ ID NO: 70 - DNA, 20 bp, Artificial Sequence
  source: /note=“Description of Artificial Sequence Synthetic primer”
ctccacttggtggtttgcta

SEQ ID NO: 71 - DNA, 20 bp, Artificial Sequence
  source: /note=“Description of Artificial Sequence Synthetic primer”
acgggtcaacttcacattca

SEQ ID NO: 72 - DNA, 21 bp, Artificial Sequence
  source: /note=“Description of Artificial Sequence Synthetic primer”
tcgagtgacaaacacgactgc

SEQ ID NO: 73 - DNA, 20 bp, Artificial Sequence
  source: /note=“Description of Artificial Sequence Synthetic primer”
ccagtttggtagcatccatc

SEQ ID NO: 74 - DNA, 21 bp, Artificial Sequence
  source: /note=“Description of Artificial Sequence Synthetic primer”
aaccctctgcacccagttttc

SEQ ID NO: 75 - DNA, 20 bp, Artificial Sequence
  source: /note=“Description of Artificial Sequence Synthetic primer”
gtgctcatgtcctcatcctg

SEQ ID NO: 76 - DNA, 20 bp, Artificial Sequence
  source: /note=“Description of Artificial Sequence Synthetic primer”
tttcggccctgagatgttcc

SEQ ID NO: 77 - DNA, 21 bp, Artificial Sequence
  source: /note=“Description of Artificial Sequence Synthetic primer”
tgcagcaccaccagcgtgtcc

SEQ ID NO: 78 - DNA, 20 bp, Artificial Sequence
  source: /note=“Description of Artificial Sequence Synthetic primer”
gacggacacattgggggtag